(19) United States
(12) Patent Application Publication
(10) Pub. No.: US 2022/0362145 A1
(43) Pub. Date: Nov. 17, 2022

(54) THERAPEUTIC HYDROGEL COMPOSITIONS

(71) Applicant: THE UNIVERSITY OF BIRMINGHAM, Birmingham (GB)

(72) Inventors: Liam GROVER, Birmingham, West Midlands (GB); Anthony METCALFE, Birmingham, West Midlands (GB); Richard WILLIAMS, Birmingham, West Midlands (GB); Richard MOAKES, Birmingham, West Midlands (GB)

(21) Appl. No.: 17/311,534

(22) PCT Filed: Dec. 9, 2019
(86) PCT No.: PCT/GB2019/053476

§ 371 Date: Jun. 7, 2021

(30) Foreign Application Priority Data
Dec. 7, 2018 (GB) 1820018.8

(51) Int. Cl. A61K 9/00; A61K 47/36

(52) U.S. Cl. CPC A61K 9/0048; A61K 47/36

(57) ABSTRACT

Disclosed are shear-thinning hydrogel compositions comprising 0.1 to 5 wt. % (such as 0.1 to 2.5 wt. %) of a microgel particle-forming polymer; and 0.5 to 100 mM of a monovalent and/or polyvalent metal ion salt as a cross-linking agent. The microgel particle-forming polymer is dispersed in an aqueous vehicle, and the hydrogel composition has a pH within the range of 3 to 8. The viscosity of the gel composition reduces when the gel is exposed to shear. Also disclosed are methods of making such compositions, and medical uses of such compositions.

### CROSS-REFERENCE TO RELATED APPLICATIONS

This application is a U.S. National Phase Application of PCT/GB2019/053476, filed Dec. 9, 2019, which claims priority to GB 1820018.8, filed Dec. 7, 2018, the contents of which applications are incorporated herein by reference in their entireties for all purposes.

### FIELD OF THE INVENTION

The present invention relates to hydrogel compositions that are useful for therapeutic applications. The present invention further relates to method for preparing these hydrogel compositions and their use for therapeutic applications, especially ocular and topical therapeutic applications.

### BACKGROUND

In 2018 the WHO reported corneal opacity to be a leading cause of blindness globally. Corneal infection caused by conditions such as microbial keratitis result in disorganization of collagen and extracellular matrix to form a scar. Treatment often requires resolution of the infection with steroids and antibiotics. Unresolved corneal opacities may lead to the need of a surgical corneal transplant. Although attempts are made to control corneal scarring through aggressive control of infection/inflammation, there has been little success for the use of potent anti-scarring treatments. One limitation of current eye drop treatments is the low viscosity or weak gelling materials, which do not significantly enhance the retention time of the drug.

Corneal opacity is a leading cause of sight impairment worldwide with an estimated 27.9 million people globally being bilaterally or unilaterally affected^{[1]}. Such opacity is typically derived from alteration of the complex, optically clear, corneal tissue structure, vital for refraction of light onto the retina, and subsequent neuro-visual processing. Commonly, corneal scarring results from ocular infections from a range of pathogens including bacteria, parasites, fungi, viruses and protozoa. In the developed world, devastating corneal infections are most commonly associated with prolonged contact lens wear and/or poor lens hygiene^{[2-4]}; with Pseudomonas aeruginosa being a prominent causative organism. In cases of gram-negative infections, e.g. Pseudomonas, the structural integrity of the cornea becomes compromised through multiple virulence factors, whereby the microbes invade epithelial cells, resulting in activation of numerous inflammatory pathways. Subsequent inflammation, neovascularization, cellular alterations and degradative stromal processes^{[5]} lead to disruption of the intricately arranged collagen fibrils^{[6]}. Continued inflammation leads to fibrosis and dysregulated remodeling of the stromal tissue matrix, with wider, disorganized collagen fibrils and loss of optical transparency, impairment of light refraction and loss of sight.

Typically, transforming growth factor beta (TGFβ) is mostly restricted to the epithelium in a healthy cornea, whereas, local trauma induces production of cytokines, including TGFβ, within the epithelium and the stroma^{[7]}. If in the injured cornea, physiological resolution, or that aided by exogenous pharmaco-manipulation, is insufficient to curb the inflammatory response, the disorganized fibril arrangements and dysregulated extracellular matrix (ECM) deposition leads to fibrotic responses and permanent corneal scarring with visual disability. Mechanistically, TGFβ activates corneal fibroblasts (keratocytes) resulting in differentiation into myofibroblasts, facilitating wound contraction via excretion of ECM molecules including collagen^{[7]}.

Currently, the standard of clinical care for patients infected with bacterial keratitis focuses initially on sterilizing the infected eye, by eye drop administration of intensive broad-spectrum antibiotics, followed by the addition of topical corticosteroids to reduce inflammation^{[8, 9]}. This is followed by strategies to limit scar formation ranging from intensive lubrication (to reduce biomechanical trauma of the eyelids abrading the wound bed during blinking), to the use of systemic pharmacological agents (sub-antimicrobial dose of tetracyclines for matrix metallo-proteinase inhibition^{[10]}) or supplements (vitamin C used as anti-oxidants and free radical scavengers^{[11]}) in an attempt to promote tissue remodeling. Unfortunately, although effective at sterilizing the eye, the patient is often left with a high degree of corneal hazing which, if it compromises the visual axis, causes loss of visual acuity. Surgical interventions to treat unresponsive and large corneal defects include either application of amniotic membrane as a biologically active bandage releasing anti-inflammatory and anti-fibrotic factors to enhance re-epithelialization and wound healing during acute injury^{[12-14]}, or in established cases of visually significant central corneal scars, excision of the scarred tissue and replacement with donor cornea. Reproducibility and repeatability of the clinical outcomes of amnion grafting and corneal transplantation are fraught with risks of failure and rejection^{[15-18]}.

If the fibrotic response to injury and infection could be attenuated, it will maximize optical clarity and preserve visual function, and may remove the need for surgical intervention and transplantation. Such an innovation would have the potential to prevent permanent sight-loss in many millions of individuals. As discussed above, fibrosis is driven by raised levels of TGFβ-1 activity and so it may be possible to prevent fibrosis using a TGFβ. antagonist. Decorin is a naturally occurring anti-fibrotic small leucine-rich proteoglycan that is naturally present at high levels bound to collagen in the corneal stroma²² and which, when released, tightly regulates TGFβ activity by binding the growth factor and sequestering it within the ECM^{[19]}. Decorin regulates cell proliferation, survival and differentiation by modulating numerous growth factors^{[20-24]} including TGFβ{Zηανγ, 2007 #28}, as well as directly interfering with collagen fibrillogenesis^{[25-28]}. Human recombinant (hr)Decorin is now available in GMP form and it has been shown is functional in minimizing fibrosis in the brain and spinal cord^{[29-31]}. To date, there has been no reported efficacy of soluble decorin being applied to the surface of the eye for treatment in vivo. One of the possible reasons for this maybe the relatively rapid clearance of eye drops (in the range of minutes^{[32, 33]}), owing to their relatively low viscosities, from the surface of the cornea at early time points, meaning that any efficacy of decorin would be limited.

### BRIEF SUMMARY OF THE DISCLOSURE

In a first aspect of the invention there is provided a shear-thinning hydrogel composition comprising:
(i) 0.1 to 5.0 wt. % (e.g. 0.1 to 3.5 wt. %, 0.1 to 2.5 wt. %) of a microgel particle-forming polymer; and
(ii) 0.5 to 100 mM of a monovalent and/or polyvalent metal ion salt as a cross-linking agent;
   dispersed in an aqueous vehicle;
   and wherein the hydrogel composition has a pH within the range of 3 to 8 and the viscosity of the hydrogel composition reduces when the hydrogel is exposed to shear.

In a further aspect of the invention, the shear-thinning hydrogel composition is an ocular hydrogel composition suitable for application to the eye. In a further aspect of the invention there is provided an ocular hydrogel composition suitable for application to the eye, wherein the ocular hydrogel composition comprises, consist essentially of, or consists of, a shear-thinning hydrogel composition as defined herein.

In another aspect, the shear-thinning hydrogel composition is a topical hydrogel composition suitable for application to a surface of the body. In a further aspect of the invention there is provided a topical hydrogel composition suitable for application to a surface of the body, wherein the topical hydrogel composition comprises, consist essentially of, or consists of, a shear-thinning hydrogel composition as defined herein.

In a further aspect the present invention provides a method of making a shear-thinning hydrogel composition as defined herein, the method comprising the steps of:
a) dissolving a microgel-forming polymer in an aqueous vehicle to form a polymer solution;
b) mixing the microgel-forming polymer solution formed in step (a) with an aqueous solution of a monovalent or polyvalent metal ion salt at a temperature above the gelling temperature of the microgel particle-forming polymer; and
c) cooling the resultant mixture from step b) to a temperature below the gelling temperature of the microgel particle-forming polymer.

In a further aspect the present invention provides a method of making a shear-thinning hydrogel composition as defined herein, the method comprising the steps of:
a) dissolving a microgel-forming polymer in an aqueous vehicle comprising 0.5 to 100 mM of a monovalent and/or polyvalent metal ion salt as a cross-linking agent to form a polymer solution comprising 0.1 to 5.0 wt. % (e.g. 0.1 to 3.5 wt. % or 0.1 to 2.5 wt. %) of the microgel particle-forming polymer;
b) mixing the microgel-forming polymer solution formed in step (a) at a temperature above the gelling temperature of the microgel particle-forming polymer; and
c) cooling the resultant mixture from step b) under shear mixing to a temperature below the gelling temperature of the microgel particle-forming polymer.

In a further aspect the present invention provides a shear-thinning hydrogel composition obtainable by, obtained by, or directly obtained by, any of the preparatory methods defined herein.

In a further aspect the present invention provides a shear-thinning hydrogel composition as defined herein for use in therapy.

In a further aspect the present invention provides a shear-thinning hydrogel composition as defined herein for ocular or topical administration.

In a further aspect the present invention provides a shear-thinning hydrogel composition as defined herein for use in the inhibition of scarring.

In a further aspect the present invention provides a shear-thinning hydrogel composition as defined herein for use in the treatment of microbial keratitis.

In a further aspect the present invention provides a shear-thinning hydrogel composition as defined herein for administration to a dermal wound.

In a further aspect the present invention provides a shear-thinning hydrogel composition as defined herein for use in the treatment of glaucoma by administration to the eye.

In a further aspect, the invention provides a composition in accordance with the invention for use as a medicament. Examples of suitable medical uses of the compositions of the invention are described further below. Suitably, the compositions of the invention may be used as topical medicaments.

In a further aspect, the invention provides a composition in accordance with the invention for use in the inhibition of scarring

In a suitable embodiment of the invention, a composition in accordance with the invention is for use in the inhibition of scarring in the eye.

### BRIEF DESCRIPTION OF THE DRAWINGS

Embodiments of the invention are further described hereinafter with reference to the accompanying drawings, in which:

FIG. 1. Processing and intrinsic material properties of the gellan based fluid hydrogel eye drop. (a) Schematic showing the production of the fluid gel: where the initial sol is continuously processed under shear whilst being cooled to form “ribbon-like” gelled entities shown using (i) transmission microscopy and (ii) scanning electron microscopy. (b) Time dependent viscosity profiles obtained for the gellan eye drop, highlighting a degree of thixotropy. (c) The fluid gel being dispensed from the eye dropper packaging (gel has been stained blue so as to be visible in the photograph). (d) Small deformation rheology data obtained at a single frequency (1 Hz, 0.5% strain) as a function of time. Data shows the evolution of an elastic network post-shearing resulting in a transition from liquid to solid-like behaviour. (e) Anterior segment OCT images showing the ocular surface before fluid gel application (top image) and post-application (bottom image). Images demonstrate a uniform layer that covers the entirety of the ocular surface.

FIG. 2. In vitro assays demonstrating the formulated eye drop's bioactivity. (a) Cumulative release curve for hrDecorin loaded eye drops over 4 hours (240 mins). Line of best fit follows a power function, y=0.7x^{0.7} (R²=0.99). (b) Collagen fibrillogenesis turbidity data for PBS control, collagen only and collagen+hrDecorin. (c) Collagen fibrillogenesis turbidity data with a dose response curve for collagen, collagen+hrDecorin, collagen+fluid gel (FG) only, collagen+hrDecorin loaded fluid gel (DecFG).

FIG. 3. Corneal opacity area measurements. (a) Representative photographs taken at days 2, 3, 9, 12, and 16 post-Pseudomonas infection and treatment. (b) Graph to show the mean area±SEM (mm²) of opacity as measured by two independent masked ophthalmologists from photographs (represented in panel a) taken from each individual mouse per group (n=6; **p<0.01, ***p<0.001).

FIG. 4. Corneal re-epithelialization. (a) Representative images of DAPI⁺ cell nuclei (blue) in the cornea used to assess the epithelium, illustrating the thickness and stratification (number of cell layers) of the epithelium in: naïve intact eyes showing normal non-keratinized stratified (approx. 5 layers) epithelium; eyes taken at day 2 after infection, which was associated with a thickened edematous stroma with cellular infiltrate; and eyes taken at 16 days post-treatment showing re-epithelization with a 2-3 layer stratification accompanied by a reduction in the stromal edemas in Group 1 (Gentamicin and Prednisolone), increased stratification in Group 2 (G.P.FG) and, fully mature epithelium in Group 3 (G.P.DecFG) (scale bar 100 μm). (b) Quantitation of corneal thickness±SEM, (c) Quantitation of epithelial layer thickness±SEM, and (d) Quantitation of cellular epithelial stratification layers±SEM in naïve intact, (n=6), with eyes evaluated at day 2, and at day 16 from each treatment group (n=6 for each group). All quantification was performed on masked images unknown to the observer.

FIG. 5. Extracellular matrix levels in the cornea. Representative images of immunohistochemical staining with accompanying plots quantifying the IR for: (a) αSMA⁺ (green to stain myofibroblasts), (b) IR fibronectin⁺ (green to stain fibronectin in the ECM), and (c) laminin⁺ (red to stain laminin in the ECM), in each case DAPI⁺ was used to stain the cell nuclei (blue). Analysis was undertaken on intact eyes, eyes taken at 2 days post-infection and eyes obtained after 16 days with various eye drop treatments: i) Gentamicin and Prednisolone (G.P), ii) Gentamicin, Prednisolone and fluid gel (G.P.FG), and iii) Gentamicin, Prednisolone and hrDecorin fluid gel (G.P.DecFG). All studies were done using n=6 treatment groups, with quantification performed on masked images unknown to the observer (scale bar=100 μm).

FIG. 6. In vivo experimental design. Experimental design for the in vivo Pseudomonas keratitis study in which the fluid gel eye drops with and without hrDecorin were compared to Gentamicin and Prednisolone eye drops alone.

FIG. 7: Storage modulus (G′) representing the elastic structure within the gellan microgel suspensions, as a function of initial gellan polymer concentration;

determined using amplitude sweeps. (a) strain sweeps obtained at 1 Hz (20° C.) for varying polymer concentrations prepared at a processing rate of 500 rpm. (b) strain sweeps obtained at 1 Hz (20° C.) for varying polymer concentrations at a processing rate of 1000 rpm.

FIG. 8: Comparison of storage moduli as a function of polymer concentration and processing speeds. G′ obtained within the linear viscoelastic region (LVR) of the amplitude sweeps shown in FIG. 7

FIG. 9: Comparison in storage moduli for commercially available eye drops/ointments for the treatment of dry eye. Data obtained from amplitude sweeps undertaken using the same method as described for gellan suspensions. Again, values were obtained within the LVR. Dotted line represents G′ for the optimised gellan formulation.

FIG. 10: Flow profiles representing the ease of application for the gellan microgel suspensions, as a function of initial gellan polymer concentration. (a) Viscosity sweeps obtained at 20° C. between 0.1 and 600 s⁻¹ for varying polymer concentrations prepared at a processing rate of 500 rpm. (b) Viscosity sweeps obtained at 20° C. between 0.1 and 600 s⁻¹ for varying polymer concentrations prepared at a processing rate of 1000 rpm.

FIG. 11: Comparison of the microgel suspension viscosity at 1 s⁻¹ a function of polymer concentration and processing speeds. Instantaneous viscosity was obtained by measuring the value at 1 s⁻¹ using the sweeps shown in FIG. 10.

FIG. 12: Comparison in viscosities at 1 s⁻¹ for commercially available eye drops/ointments for the treatment of dry eye. Data obtained from flow profiles undertaken using the same method as described for gellan suspensions. Dotted line represents the viscosity of the optimised gellan formulation.

FIG. 13: Storage modulus (G′) representing the elastic structure within the gellan microgel suspensions, as a function of cross-linker added; determined using amplitude sweeps. (a) strain sweeps obtained at 1 Hz (20° C.) for varying cross-linker concentrations for 0.9% (w/v) systems. (b) strain sweeps obtained at 1 Hz (20° C.) for varying cross-linker concentrations for 1.8% (w/v) polymer concentrations.

FIG. 14: Comparison of storage moduli as a function the cross-linker and polymer concentrations. G′ obtained within the linear viscoelastic region (LVR) of the amplitude sweeps shown in FIG. 7.

FIG. 15: Flow profiles representing the ease of application for the gellan microgel suspensions, as a function of the cross-linker concentration. (Left) Viscosity sweeps obtained at 20° C. between 0.1 and 600 s⁻¹ for 0.9% (w/v) gellan systems prepared with varying concentrations of cross-linker. (Right) Viscosity sweeps obtained at 20° C. between 0.1 and 600 s⁻ for 1.8% (w/v) gellan systems prepared with varying concentrations of cross-linker.

FIG. 16: Comparison of the microgel suspension viscosities at 1 s⁻¹ as a function of the polymer and cross-linker concentrations. Instantaneous viscosity was obtained by measuring the value at 1 s⁻¹ using the sweeps shown in FIG. 3.

FIG. 17: Storage modulus (G′) representing the elastic structure within the gellan microgel suspensions, as a function of cooling rate applied during processing; determined using amplitude sweeps. (a) strain sweeps obtained at 1 Hz (20° C.) for varying cooling rates for 0.9% (w/v) systems prepared at a processing rate of 1000 rpm. (b) strain sweeps obtained at 1 Hz (20° C.) for varying cooling rates for 1.8% (w/v) polymer concentrations at a processing rate of 1000 rpm.

FIG. 18: Comparison of storage moduli as a function of cooling rate and polymer concentration. G′ obtained within the linear viscoelastic region (LVR) of the amplitude sweeps shown in FIG. 7.

FIG. 19: Flow profiles representing the ease of application for the gellan microgel suspensions, as a function of the cooling rate applied during processing. (a) Viscosity sweeps obtained at 20° C. between 0.1 and 600 s⁻¹ for 0.9% (w/v) gellan systems prepared at various cooling rates. (b) Viscosity sweeps obtained at 20° C. between 0.1 and 600 s⁻ for 1.8% (w/v) gellan systems prepared at various cooling rates.

FIG. 20: Comparison of the microgel suspension viscosities at 1 s⁻¹ as a function of polymer concentration and cooling rate applied during processing. Instantaneous viscosity was obtained by measuring the value at 1 s⁻¹ using the sweeps shown in FIG. 9.

FIG. 21: Storage modulus (G′) representing the elastic structure within the gellan microgel suspensions, as a function of mechanical shear applied during processing; determined using amplitude sweeps. (a) strain sweeps obtained at 1 Hz (20° C.) for varying processing speeds for 0.9% (w/v) systems. (b) strain sweeps obtained at 1 Hz (20° C.) for varying processing speeds for 1.8% (w/v) polymer concentrations.

FIG. 22: Comparison of storage moduli as a function of processing speeds and polymer concentration. G′ obtained within the linear viscoelastic region (LVR) of the amplitude sweeps shown in FIG. 7.

FIG. 23: Flow profiles representing the ease of application for the gellan microgel suspensions, as a function of the mechanical shear applied during processing. (a) Viscosity sweeps obtained at 20° C. between 0.1 and 600 s⁻¹ for 0.9% (w/v) gellan systems prepared at various processing speeds. (b) Viscosity sweeps obtained at 20° C. between 0.1 and 600 s⁻ for 1.8% (w/v) gellan systems prepared at various processing speeds.

FIG. 24: Comparison of the microgel suspension viscosities at 1 s⁻¹ as a function of polymer concentration and processing speeds during gelation. Instantaneous viscosity was obtained by measuring the value at 1 s⁻¹ using the sweeps shown in FIG. 9.

FIG. 25: shear-thinning hydrogel compositions in accordance with the invention reduce expression in cultured fibroblasts of markers associated with scarring. Administration of TGF-β to cultured human dermal fibroblasts increases expression of α-smooth muscle actin, a marker of myofibroblasts associated with scarring. Graphs show the impact of treatment with experimental hydrogel compositions on this expression. Hydrogel compositions with or without the anti-fibrotic agent decorin are able to reduce expression of α-sma, indicating an ability to inhibit scarring.

FIG. 26: Amplitude sweep data obtained for agar, gellan, kappa carrageenan and alginate.

FIG. 27: Frequency sweep data obtained for agar, gellan, kappa carrageenan and alginate

FIG. 28: Viscosity sweep data obtained for agar, gellan, kappa carrageenan and alginate

FIG. 29 illustrates standard curves obtained in respect of shear-thinning hydrogel compositions in accordance with the invention incorporating the following active agents: penicillin-streptomycin; dexamethasone; proteinase K; ibuprofen; dextran; and dextran blue.

FIG. 30 illustrate curves obtained in respect of shear-thinning hydrogel compositions in accordance with the invention incorporating the following active agents: penicillin-streptomycin; dexamethasone; proteinase K; ibuprofen; dextran; and dextran blue.

FIG. 31 shows photographs illustrating the results of zone of inhibition assays using shear-thinning hydrogel compositions in accordance with the invention comprising the polymers alginate or gellan, in combination with an anti-infective agent (penicillin-streptomycin). These results demonstrate effectiveness in respect of E. coli and S. aureus. A summary of the results is also provided in the accompanying Table. The Figure also includes a graph illustrating the results of zone of inhibition assays using shear-thinning hydrogel compositions in accordance with the invention comprising alginate in combination with an alternative anti-infective agent (vancomycin). Anti-microbial effectiveness of vancomycin was tested against MRSA.

FIG. 32 shows photographs demonstrating breakdown over time of the exemplary ECM molecule fibrin (shown as a white gel in the photographs) under the action of the active agent proteinase K released from alginate or gellan shear-thinning hydrogel compositions in accordance with the invention.

FIG. 33 is a graph illustrating the results of this study, and comparing absorbance at 405 nm (y-axis) for collagen alone (“collagen only”), or collagen incubated with decorin alone “hrDecorin”) or with increasing concentrations of gellan fluid gel shear-thinning hydrogel compositions of the invention with (“DecFG”) or without (“FG”) human recombinant decorin (Galacorin™).

FIG. 34 represents the mouse model used to study effect of compositions of the invention on experimental microbial keratitis.

FIG. 35 sets out graphs showing the area of opacity associated with the different treatments at different timepoints, the percentage of α-smooth muscle actin pixels above the threshold value for the various control and treatment groups investigated, the percentage of fibronectin pixels above the threshold value for the various control and treatment groups investigated, and the percentage of laminin pixels above the threshold value for the various control and treatment groups investigated.

FIG. 36 shows results of a study of intraocular pressure in hypertensive rats, where treated rats are shown in dashed line, and non-treated controls with a solid black line. Results were analysed with two-way ANOVA, with Sidak multiple comparisons tests and demonstrated that the composition of the invention (p<0.05) significantly reduced intraocular pressure in ocular hypertensive rats by D28 compared to controls.

### DETAILED DESCRIPTION

### Definitions

The term “hydrogel” is used herein to refer to a gel formed from a hydrophilic polymer dispersed within an aqueous vehicle.

The term “aqueous vehicle” is used herein to refer to water or water-based fluid (e.g. a buffer such as, for example, phosphate buffered saline or a physiological fluid such as, for example, serum).

The term “microgel” is used herein to refer to a microscopic particle of gel formed from a network of microscopic filaments of polymer.

The term “shear-thinning” is used herein to define the hydrogel compositions of the present invention. This terminology is well understood in the art and refers to hydrogel compositions that have a viscosity that reduces when a shear force is applied to the hydrogel. The shear-thinning hydrogel compositions of the invention possess a “resting” viscosity (in the absence of any applied shear force), and a lower viscosity when a shear force is applied. This property of hydrogel compositions enables them to flow and be administered to the body when a shear force is applied (for example, by applying a force to a tube or dispenser containing the hydrogel composition of the invention). Once applied under the application of shear, and the applied shear force is removed, the viscosity of hydrogel composition increases. Typically, the hydrogel compositions of the present invention will have a viscosity of below 1 Pa·s when subjected to a shear force to administer the hydrogel composition. At viscosities below 1 Pa·s, the hydrogel composition will be capable of flowing. The resting viscosity will typically be above 1 Pa·s, for example greater than 2 Pa·s, greater than 3 Pa·s, or greater than 4 Pa·s.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Throughout the description and claims of this specification, the words “comprise” and “contain” and variations of them mean “including but not limited to”, and they are not intended to (and do not) exclude other additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The reader's attention is directed to all papers and documents which are filed concurrently with or previous to this specification in connection with this application and which are open to public inspection with this specification, and the contents of all such papers and documents are incorporated herein by reference.

### Hydrogel Compositions of the Invention

In a first aspect of the invention there is provided a shear-thinning hydrogel composition comprising:
(i) 0.1 to 5.0 wt. % (e.g. 0.1 to 3.5 wt. % or 0.1 to 2.5 wt. %) of a microgel particle-forming polymer; and
(ii) 0.5 to 100 mM of a monovalent and/or polyvalent metal ion salt as a cross-linking agent;
   dispersed in an aqueous vehicle;
   and wherein the hydrogel composition has a pH within the range of 3 to 8 and the viscosity of the hydrogel composition reduces when the hydrogel is exposed to shear.

The hydrogel compositions of the present invention are shear-thinning, meaning that the viscosity of the composition reduces when the hydrogel is exposed to shear. This property enables the hydrogels to reduce in viscosity and flow when a shear force is applied, thereby enabling them to be dispensed and administered, for example from an eye dropper to tube, by applying a shear force (e.g. by squeezing the sides of the eye dropper or tube). Once administered and the shear force applied to the hydrogel diminishes, the viscosity of the hydrogel increases to form a thicker gel capable of residing at the point of administration for a prolonged period.

Typically, the hydrogel compositions of the present invention will have a viscosity of below 1 Pa·s when subjected to a shear force to administer the hydrogel composition. At viscosities below 1 Pa·s, the hydrogel composition will be capable of flowing. The resting viscosity will typically be above 1 Pa·s, for example greater than 2 Pa·s, greater than 3 Pa·s, or greater than 4 Pa·s.

In an embodiment, the shear-thinning hydrogel compositions of the present invention do not comprise collagen and/or fibrin.

The microgel particle-forming polymer may be any polymer that is capable of forming microgel particles in the aqueous vehicle. The microgel particles formed by the microgel particle-forming polymer may have any suitable morphology (e.g. they may be linear filaments or regular or irregular shaped particles) and/or particle size. The formation of microgel particles, as opposed to a macrogel structure, facilitates the desired shear-thinning characteristics. Without wishing to be bound by any particular theory, it is postulated that, in the absence of shear or at low levels of shear, the microgel particles are bound together, substantially impeding the bulk flow of the hydrogel. However, upon the application of a shear force, the interactions between adjacent microgel particles are overcome, and the viscosity decreases, thereby enabling the hydrogel composition to flow. Once the applied shear force is removed, then the interactions between adjacent microgel particles can reform such that the viscosity increases again and the ability to flow readily is impeded.

Suitably, the hydrogel composition comprises 0.5 to 5.0 wt. % of the microgel particle-forming polymer. In an embodiment, the hydrogel composition comprises 0.5 to 3.5 wt. % of the microgel particle-forming polymer. In an embodiment, the hydrogel composition comprises 0.5 to 2.5 wt. % of the microgel particle-forming polymer. In an embodiment, the hydrogel composition comprises 0.8 to 1.8 wt. % of microgel particle-forming polymer. In a further embodiment, the hydrogel composition comprises 0.8 to 1.0 wt. % (e.g. 0.9 wt. %) of a microgel particle-forming polymer.

Suitably, the microgel particle-forming polymer is one or more polysaccharide microgel particle-forming polymers. In an embodiment, the microgel particle-forming polymer is selected from one or more of the following groups: gellans, alginates, carrageenans (e.g. iota-carrageenan, kappa-carrageenan), agar, agarose, or chitosan. In a particular embodiment, the microgel particle-forming polymer is selected from one or more of the following groups: agars, gellans, alginates or carrageenans. In a particular embodiment, the microgel particle-forming polymer is selected from one or more of the following groups: gellans, alginates or carrageenans. In a more particular embodiment, the microgel particle-forming polymer is selected from gellan or alginate. In yet another embodiment, the microgel particle-forming polymer is gellan. In yet another embodiment, the microgel particle-forming polymer is an alginate.

In an alternative embodiment, the microgel particle-forming polymer is gelatin.

Suitably, the hydrogel composition is transparent or translucent. In a particular embodiment, the hydrogel composition is transparent.

In an embodiment, the hydrogel composition is transparent or translucent and the microgel particle-forming polymer is selected from gellans, alginates and/or carrageenans.

In a further embodiment, the hydrogel composition is transparent and the microgel particle-forming polymer is selected from gellans, alginates and/or carrageenans. In a particular embodiment, the hydrogel composition is transparent and the microgel particle-forming polymer is gellan or alginate. In a further embodiment, the hydrogel composition is transparent and the microgel particle-forming polymer is gellan.

Gelan (also referred to gellan gum) is a water-soluble anionic polysaccharide produced by the bacterium Sphingornonas elodea. It is commercially available in a low acyl form under the trade name Kelco gel (Kelco gel CG LA, Azelis, UK).

The hydrogel composition comprises 5 to 100 mM of a monovalent and/or polyvalent metal ion salt as a cross-linking agent. The metal ion salt may be added to the composition as a component, but it may also be present in other components of the composition, e.g. components such as buffers (e.g. phosphate buffered saline) or any physiological fluids present in the composition, such as, for example, serum.

Suitably, the hydrogel composition comprises 5 to 40 mM of a monovalent and/or polyvalent metal ion salt as a cross-linking agent. In an embodiment, the hydrogel composition comprises 5 to 30 mM of a monovalent and/or polyvalent metal ion salt as a cross-linking agent. In another embodiment, the hydrogel composition comprises 5 to 20 mM of a monovalent and/or polyvalent metal ion salt as a cross-linking agent. In yet another embodiment, the hydrogel composition comprises 5 to 15 mM of a monovalent and/or polyvalent metal ion salt as a cross-linking agent. In yet another embodiment, the hydrogel composition comprises 8 to 12 mM (e.g. 10 mM) of a monovalent and/or polyvalent metal ion salt as a cross-linking agent.

In a particular embodiment of the invention, the microgel particle-forming polymer is gellan and the composition comprises 0.5 to 40 mM, 5 to 15 mM, 8 to 12 mM or 10 mM of a monovalent metal ion salt (e.g. NaCl) as a cross-linking agent.

In a further embodiment of the invention, the microgel particle-forming polymer is alginate and the composition comprises 0.5 to 40 mM, 5 to 15 mM, 8 to 12 mM or 10 mM of a polyvalent metal ion salt (e.g. a Ca²⁺ salt) as a cross-linking agent.

Suitably, the hydrogel composition has a pH within the range of 6 to 8. In an embodiment, the hydrogel composition has a pH within the range of 6.5 to 8. In a further embodiment, the hydrogel composition has a pH within the range of 7 to 7.5 (e.g. pH 7.4).

Suitably, the hydrogel composition of the present invention has a resting viscosity (i.e. a viscosity at zero shear) of 1 Pa·s or greater (e.g. 1 Pa·s to 200 Pa·s or 1 Pa·s to 100 Pa·s). More suitably, the resting viscosity will be 2 Pa·s or greater (e.g. 2 Pa·s to 200 Pa·s or 2 Pa·s to 100 Pa·s), 3 Pa·s or greater (e.g. 3 Pa·s to 200 Pa·s or 3 Pa·s to 100 Pa·s), 4 Pa·s or greater (e.g. 4 Pa·s to 200 Pa·s or 4 Pa·s to 100 Pa·s), or 5 Pa·s or greater (e.g. 5 Pa·s to 200 Pa·s or 5 Pa·s to 100 Pa·s).

The viscosity reduces when the hydrogel composition is subjected to a shear force. Suitably, the viscosity reduces to a value below the resting viscosity at which the gel can flow and be administered. Typically, the viscosity will reduce to a value of less than 1 Pa·s when a shear force is applied.

In an embodiment, the hydrogel composition has a resting viscosity of 1 Pa·s or greater (e.g. 1 Pa·s to 200 Pa·s or 1 Pa·s to 100 Pa·s) and when subject to a shear force, the viscosity reduces to below 1 Pa·s.

In another embodiment, the hydrogel composition has a resting viscosity of 2 Pa·s or greater (e.g. 2 Pa·s to 200 Pa·s or 2 Pa·s to 100 Pa·s) and when subject to a shear force, the viscosity reduces to below 2 Pa·s (for example, to below 1 Pa·s).

In another embodiment, the hydrogel composition has a resting viscosity of 3 Pa·s or greater (e.g. 3 Pa·s to 200 Pa·s or 3 Pa·s to 100 Pa·s) and when subject to a shear force, the viscosity reduces to below 3 Pa·s (for example, to below 1 Pa·s).

In another embodiment, the hydrogel composition has a resting viscosity of 4 Pa·s or greater (e.g. 4 Pa·s to 200 Pa·s or 4 Pa·s to 100 Pa·s) and when subject to a shear force, the viscosity reduces to below 4 Pa·s (for example, to below 1 Pa·s).

In another embodiment, the hydrogel composition has a resting viscosity of 5 Pa·s or greater (e.g. 5 Pa·s to 200 Pa·s or 5 Pa·s to 100 Pa·s) and when subject to a shear force, the viscosity reduces to below 5 Pa·s (for example, to below 1 Pa·s).

For the avoidance of doubt, all viscosity values quoted herein are quoted at a normal ambient temperature of 20° C. The viscosity of hydrogel compositions of the present invention can be determined using standard techniques well known in the art. For example, viscosity profiles can be obtained using an AR-G2 (TA Instruments, UK) rheometer equipped with sandblasted parallel plates (40 mm, 1 mm gap height) at 20° C.

Suitably, the hydrogel has an elastic modulus of 5 Pa to 40 Pa at zero shear.

The elastic modulus of the hydrogels of the present invention can be determined by techniques well known in the art.

### Particular Embodiments

Particular embodiments of the invention include those in which the shear-thinning hydrogel composition comprises:
(I) 0.1 to 5.0 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 3.5 to 8.
(II) 0.1 to 5.0 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6 to 8.
(III) 0.1 to 5.0 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6.5 to 7.5.
(IV) 0.1 to 3.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 3.5 to 8.
(V) 0.1 to 3.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6 to 8.
(VI) 0.1 to 3.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6.5 to 7.5.
(1) 0.1 to 2.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 3.5 to 8.
(2) 0.1 to 2.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6 to 8.
(3) 0.1 to 2.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6.5 to 7.5.
(4) 0.5 to 2.0 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 3.5 to 8.
(5) 0.8 to 1.8 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6 to 8.
(6) 0.8 to 1.0 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6.5 to 7.5.
(7) 0.5 to 2.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 3.5 to 8.
(8) 0.5 to 2.5 wt. % of a microgel particle-forming polymer (e.g. gellan);
   5 to 20 mM of a monovalent metal ion salt (e.g. NaCl) or polyvalent metal ion salt (e.g. Ca²⁺) as a cross-linking agent; and
   the hydrogel composition has a pH of 6 to 8.
(9) 0.5 to 2.5 wt. % of a microgel particle-forming polymer (e.g. gellan);
   5 to 15 mM of a monovalent metal ion salt (e.g. NaCl) or polyvalent metal ion salt (e.g. Ca²⁺) as a cross-linking agent; and
   the hydrogel composition has a pH of 6 to 8.
(10) 0.5 to 2.5 wt. % of a microgel particle-forming polymer (e.g. gellan);
   8 to 12 mM (e.g. 10 mM) of a monovalent metal ion salt (e.g. NaCl) or polyvalent metal ion salt (e.g. Ca²⁺) as a cross-linking agent; and
   the hydrogel composition has a pH of 6 to 8.
(11) 0.5 to 2.5 wt. % of a microgel particle-forming polymer (e.g. gellan);
   0.5 to 40 mM of a monovalent metal ion salt (e.g. NaCl) or polyvalent metal ion salt (e.g. Ca²⁺) as a cross-linking agent; and
   the hydrogel composition has a pH of 6 to 8.
(12) 0.8 to 1.8 wt. % of a microgel particle-forming polymer (e.g. gellan);
   5 to 20 mM of a monovalent metal ion salt (e.g. NaCl) or polyvalent metal ion salt (e.g. Ca²⁺) as a cross-linking agent; and
   the hydrogel composition has a pH of 6 to 8.
(13) 0.8 to 1.0 wt. % of a microgel particle-forming polymer (e.g. gellan);
   5 to 15 mM of a monovalent metal ion salt (e.g. NaCl) or polyvalent metal ion salt (e.g. Ca²⁺) as a cross-linking agent; and
   the hydrogel composition has a pH of 6 to 8.
(14) 0.8 to 1.0 wt. % of a microgel particle-forming polymer (e.g. gellan);
   8 to 12 mM (e.g. 10 mM) of a monovalent metal ion salt (e.g. NaCl) or polyvalent metal ion salt (e.g. Ca²⁺) as a cross-linking agent; and
   the hydrogel composition has a pH of 6 to 8.

### Therapeutic Agents

In certain embodiments of the invention, the hydrogel composition may further comprise one or more pharmacologically active agents. Any suitable pharmacologically active agent may be present. For example, the hydrogel composition may comprise one or more pharmacologically active agents selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; and a pigmentation modifying agent. The anti-fibrotic agent may be decorin. It will be appreciated that in the context of the present invention when decorin is incorporated in a hydrogel composition of the invention it may be present as an active agent incorporated in the hydrogel, rather than as a constituent of the hydrogel per se.

The hydrogel composition may comprise any suitable amount of a pharmacologically active agent. For example, the hydrogel composition may comprise 0.01 to 50 wt. % of a pharmacologically active agent.

In an embodiment, the hydrogel composition comprises decorin, optionally in an amount of from 0.1 to 1.0 mg/ml; 0.1 to 0.5 mg/ml; 0.1 to 0.4 mg/ml; or 0.2 to 0.3 mg/ml.

In a further embodiment, the hydrogel composition comprises decorin, optionally in an amount of from 0.1 to 1.0 mg/ml; 0.1 to 0.5 mg/ml; 0.1 to 0.4 mg/ml; or 0.2 to 0.3 mg/ml, in any one of the hydrogel compositions defined in paragraphs (1) to (14) above.

In an embodiment of a composition of the invention comprising an anti-infective agent, such as the antibiotic gentamicin, this may be present in an amount of from 1 to 5 mg/ml. For example, an anti-infective agent, such as gentamicin, may be present in an amount of from 1 to 4 mg/ml, from 1 to 3 mg/ml, or from 1 to 2 mg/ml. An anti-infective agent, such as gentamicin, may be present in an amount of from 2 to 4 mg/ml, or from 2.5 to 3.5 mg/ml.

In an embodiment of a composition of the invention comprising an anti-inflammatory agent, such as the steroid prednisolone, this may be present in an amount of from 0.5 to 250 mg/ml. Suitably, an anti-inflammatory agent such as prednisolone may be present in an amount of from 1.25 to 170 mg/ml, for example from 1.25 to 50 mg/ml, or from 1.25 to 10 mg/ml.

### Ocular Compositions

In a further aspect, the present invention provides an ocular hydrogel composition suitable for administration to the eye, wherein the ocular hydrogel composition is a shear-thinning hydrogel composition as defined hereinbefore.

In a further aspect of the invention there is provided an ocular hydrogel composition suitable for application to the eye, wherein the ocular hydrogel composition comprises, consist essentially of, or consists of, a shear-thinning hydrogel composition as defined hereinbefore.

The ocular hydrogel compositions of the present invention are compatible with application to the eye.

### Topical Compositions

In a further aspect, the present invention provides a hydrogel composition suitable for topical administration, wherein the ocular hydrogel composition is a shear-thinning hydrogel composition as defined hereinbefore.

In a further aspect of the invention there is provided a topical hydrogel composition suitable for topical application to the body, wherein the topical hydrogel composition comprises, consist essentially of, or consists of, a shear-thinning hydrogel composition as defined hereinbefore.

### Methods of Preparing the Hydrogel Compositions of the Invention

The present invention provides a method of making a shear-thinning hydrogel composition as defined herein, the method comprising the steps of:
a) dissolving a microgel particle-forming polymer in an aqueous vehicle to form a polymer solution;
b) mixing the microgel particle-forming polymer solution formed in step (a) with an aqueous solution of a monovalent or polyvalent metal ion salt at a temperature above the gelling temperature of the microgel particle-forming polymer; and
c) cooling the resultant mixture from step b) under shear mixing to a temperature below the gelling temperature of the microgel particle-forming polymer.

Suitably, step a) is performed by heating the microgel particle-forming polymer and aqueous vehicle to a temperature above the gelling temperature for the microgel particle-forming polymer. For example, in embodiments where the microgel particle-forming polymer is gellan, the gellan/aqueous vehicle mixture may be heated to 60 to 90° C. (e.g. 70° C.) in order to dissolve the gellan polymer.

It will be appreciated that the amount of polymer dissolved will depend on the amount of polymer required in the hydrogel composition (i.e. it will be within the limits defined hereinbefore for the hydrogel composition).

In step b), the solution formed in step a) is suitably maintained at a temperature above the gelation temperature for the microgel particle-forming polymer and is mixed with an aqueous solution of a monovalent or polyvalent metal ion salt. Suitably, in step b), the solution from step a) is continuously agitated before, during and/or after the addition of the solution of the monovalent or polyvalent metal ion salt. For example, the mixture may be mixed at a rate of 50 to 2000 revolutions per minute (rpm) to ensure thorough mixing. In an embodiment, a mixing rate of 300 to 900 rpm or 500 to 800 rpm may be used. A person skilled in the art will appreciate that the mixing rate and mixing apparatus can be varied to provide a desired level of shear/agitation.

In an embodiment, where the microgel particle-forming polymer is gellan, the gellan/aqueous vehicle solution from step a) may be cooled to a temperature of, for example, 35 to 50° C. (e.g. 40° C.) prior to mixture with a monovalent cation solution.

It will be appreciated that the amount of monovalent or polyvalent metal ion salt solution added will depend on the amount of metal ion salt required in the final hydrogel composition (i.e. it will be within the limits defined hereinbefore for the hydrogel composition).

In step c), the mixture from step b) is cooled to a temperature below the gelation temperature for the microgel particle-forming polymer such that microgel particles form in the hydrogel composition. Suitably, the mixture from step b) is cooled gradually with constant mixing. In an embodiment, the mixture from step b) is cooled at a constant cooling rate with continuous agitation/shear applied. The cooling under agitation/shear may continue until the mixture reaches ambient temperature (e.g. 20° C.), at which point the final hydrogel composition may be collected and stored, for example under refrigeration conditions.

The cooling rate used in step c) and the amount of shear/agitation applied can be varied. For example, a cooling rate of 0.2 to 4° C./min, 0.5 to 3° C./min, 0.5 to 2° C./min, 0.5 to 1.5° C./min, or 1° C./min may be used. The amount of shear applied may be, for example, 50 to 2000 rpm, 300 to 900 rpm, or 400 to 500 (e.g. 450) rpm. Any suitable equipment may be used to provide the required agitation/shear. In the accompanying examples, a rotational rheometer (AR-G2, TA Instruments, UK) equipped with cup and vane geometry (cup: 35 mm diameter, vane: 28 mm diameter) is used to provide the required shear.

A pharmacologically active agent may be added:
i) during step a)
ii) during step b); or
iii) during step c) at a point wherein the mixture from step b) is at a temperature above the gelling temperature of the microgel particle-forming polymer.

Suitably, a pharmacologically active agent is added to the mixture in step b) or step c) of the method. Suitably, a pharmacologically active agent is added during step c) at a point where the mixture is above the gelling temperature for the microgel particle-forming polymer. Most suitably, the mixture from step b) is cooled to a temperature above the gelling temperature for the microgel particle-forming polymer, a pharmacologically active agent is added and thoroughly mixed into the mixture, and the mixture is then further cooled to a temperature below the gelling temperature for the microgel particle-forming polymer.

Suitably, a pharmacologically active agent is added to the mixture in either step b) or step c) in the form of an aqueous solution.

In an embodiment, the pharmacologically active agent is decorin.

In a further aspect the present invention provides a method of making a shear-thinning hydrogel composition as defined herein, the method comprising the steps of:
a) dissolving a microgel particle-forming polymer in an aqueous vehicle comprising 0.5 to 100 mM of a monovalent and/or polyvalent metal ion salt as a cross-linking agent;
b) mixing the microgel-forming polymer solution formed in step (a) at a temperature above the gelling temperature of the microgel particle-forming polymer; and
c) cooling the resultant mixture from step b) to a temperature below the gelling temperature of the microgel particle-forming polymer.

In the above aspect of the invention, the process is the same as the previous process defined above except that the microgel-particle forming polymer is dissolved directly in an aqueous vehicle comprising 0.5 to 100 mM of a monovalent and/or polyvalent metal ion salt as a cross-linking agent. The conditions and variable for steps a), b) and c) described above apply equally to this variant of the process.

In a further aspect the present invention provides a shear-thinning gel composition obtainable by, obtained by, or directly obtained by, any of the preparatory methods defined herein.

### Medical Uses of the Compositions of the Invention, and Methods of Treatment Using the Compositions of the Invention

An aspect of the invention provides compositions of the invention for use as a medicament. Compositions of the invention are suitable for medical use in the inhibition of scarring (as set out in a further aspect of the invention); as well as the prevention and/or treatment of infection; the prevention and/or treatment of pain; the prevention and/or treatment of inflammation; and the prevention and/or treatment of proliferative disorders. Compositions to be employed in such medical uses may comprise, as required, an active agent selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent; and a pigmentation modifying agent.

Without detracting from the above, the inventors have also found that compositions of the invention that do not comprise a pharmacologically active agent may be used successfully in the inhibition of scarring. Such use is demonstrated in the data presented herein.

It will be appreciated that compositions of the invention are also suitable for use in methods of medical treatment. For example, compositions of the invention may be used in methods selected from the group consisting of: methods for the inhibition of scarring; methods for the prevention and/or treatment of infection; methods for the prevention and/or treatment of pain; methods for the prevention and/or treatment of inflammation; methods for the prevention and/or treatment of proliferative disorders; methods for the prevention and/or treatment of hyperpigmentation; methods for the prevention and/or treatment of hypopigmentation; methods for inducing keratolysis; methods requiring modification of the extracellular matrix; methods requiring modification of cell junctions; and methods requiring modification of basement membranes.

In practicing such methods, a composition of the invention may be administered, as required, to a subject in need of inhibition of scarring; a subject in need of prevention and/or treatment of infection; a subject in need of prevention and/or treatment of pain; a subject in need of prevention and/or treatment of inflammation; a subject in need of prevention and/or treatment of proliferative disorders; a subject in need of prevention and/or treatment of hyperpigmentation; a subject in need of prevention and/or treatment of hypopigmentation; a subject in need of keratolysis; a subject in need of modification of the extracellular matrix; a subject in need of modification of cell junctions; and a subject in need of modification of basement membranes.

As above, compositions to be employed in such methods of treatment may comprise, as required, an active agent selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent; and a pigmentation modifying agent.

Methods for the inhibition of scarring may involve administration of a composition of the invention that does not comprise a pharmacologically active agent.

Except for where the context requires otherwise, considerations set out in the present disclosure with respect to medical uses of the compositions of the invention should also be taken as applicable to methods of treatment utilising the compositions of the invention. Similarly, considerations set out in the present disclosure with respect to methods of treatment utilising the compositions of the invention should also be taken as applicable to medical uses of the compositions of the invention.

Inhibition of Scarring

It is recognised that scarring results in deleterious effects in many clinical contexts. For example, scarring of the eye may be associated with loss of sight, and risk of blindness, while scarring in the skin may be associated with reduced mobility, discomfort, and disfigurement (which may give rise to psychological difficulties).

Scarring may also give rise to complications, and hence reduced effectiveness, in surgical procedures. Merely by way of example, scarring that occurs after surgical insertion of stents (such as for the treatment of glaucoma) may fully or partially occlude the passageway in the stent, thus rendering the surgery ineffective.

It will be appreciated that “inhibition of scarring” encompasses both partial inhibition of scarring and complete inhibition of scarring. Suitable values relating to the extent to which scarring may be inhibited in accordance with the invention are described further below.

Compositions of the invention may be useful in the inhibition of scarring or fibrosis at many body sites. Merely by way of example, the compositions of the invention may be used in the inhibition of: scarring in the eye; scarring in the skin; scarring in the muscles or tendons; scarring in the nerves; fibrosis of internal organs, such as the liver or lungs; or the formation of adhesions, such as surgical adhesions or omental adhesions.

Scarring in the eye, of the sort that may be inhibited by the medical use of compositions of the invention, includes scarring of the cornea, scarring of the retina, scarring of the ocular surface, and scarring in and around the optic nerve. Whilst the compositions of the invention are suitable for topical use, it will be appreciated that agents administered topically may have an effect on the internal anatomy. Thus, compositions administered to the surface of the eye may be effective in inhibiting intraocular scarring.

Scarring in the eye that may be inhibited by the medical use of compositions of the invention may also include scarring associated with infection, such as keratitis. Such keratitis may arise as a result of microbial infection, viral infection, parasitic infection, or fungal infection. The compositions and methods of the invention have shown particular utility in the inhibition of scarring associated with microbial keratitis.

Keratitis may also arise as a result of injury, or of disorders including autoimmune diseases such as rheumatoid arthritis or Sjogren's syndrome. The compositions and methods of the invention may also be used in inhibiting scarring associated with keratitis occurring as a result of these causes.

Scarring in the eye that may be inhibited by the medical use of compositions of the invention may also include scarring associated with surgery, such as surgery for the treatment of glaucoma (for example by the insertion of stents); and surgical procedures such as LASIK or LASEK surgery, and scarring associated with accidental injuries.

Suitably a composition of the invention for use in the inhibition of scarring may comprise gellan. Surprisingly, compositions of the invention comprising gellan are able to effectively inhibit scarring even in the absence of pharmacologically active agent, such as an active anti-fibrotic agent. That said, incorporation of an anti-fibrotic agent into a composition of the invention demonstrates beneficial properties in the inhibition of scarring. Merely by way of illustration, decorin represents an example of such an anti-fibrotic agent suitable for incorporation in compositions of the invention that are for use in the inhibition of scarring.

The skilled person will be aware of many suitable methodologies that allow the identification and quantification of scarring. These methodologies may also be used to identify inhibition of scarring. Thus they may be used to illustrate the effective medical use of the compositions of the invention, to identify therapeutically effective doses of anti-fibrotic agents, and also in the identification and/or selection of anti-fibrotic agents to be incorporated in the compositions of the invention.

The skilled person will be aware that there are many parameters by which the inhibition of scarring in the eye can be assessed. Examples of these are discussed further in the Examples. Some of these, such as induction of myofibroblast or ECM components, are also common to body sites outside the eye, while others are specific to the eye.

For example, scarring in the eye may be indicated by an increase in corneal opacity. Such an increase in corneal opacity may be demonstrated by an increase in the area of the cornea that is opaque. Thus, inhibition of scarring may be indicated by a reduction in corneal opacity as compared to a suitable control. Such a decrease in corneal opacity may be demonstrated by a decrease in the area of the cornea that is opaque.

The ability of compositions of the invention, comprising the anti-fibrotic agent decorin, to reduce corneal opacity, and to maintain such a reduction over time, is demonstrated in the data set out in the Examples.

Compositions of the invention may be used in the inhibition of scarring associated with dermal wounds. A suitable dermal wound may be selected from the group consisting of: a burn; an incision; an excision; an abrasion; a chronic wound; and a wound arising from the body's reaction to a stimulus. Examples of this latter category include systemic chemical and/or allergic reactions that cause skin to blister severely and to shed, as well as genetic-related diseases that result in compromised skin structure and homeostasis. These reactions or diseases may lead to skin blistering, peeling and dramatically increased risk and severity of wounding (even from relatively minor contact). Examples of such diseases include epidermolysis bullosa (for example epidermolysis bullosa simplex, junctional epidermolysis bullosa, or dystrophic epidermolysis bullosa) and Kindler syndrome. The compositions or methods of the invention are suitable for use in inhibition of scarring in subjects having such diseases.

Other parameters indicative of scarring may be common to a number of different tissues. For example, scarring at many body sites may be indicated by an increase in the presence of myofibroblasts. Such an increase may be demonstrated by an increase in α-smooth muscle actin expression. Thus, inhibition of scarring may be indicated by a reduction in myofibroblast numbers as compared to a suitable control. A reduction in myofibroblast numbers of this sort may be demonstrated by a decrease in α-smooth muscle actin expression.

Myofibroblasts develop at the site of injuries and are associated with progression of the scarring response. They can be characterised by their expression of α-smooth muscle actin (α-sma). Myofibroblasts can have a number of adverse effects on scar formation, including causing contractions within the healed area. The compositions of the invention are able to inhibit α-sma expression as assessed in vitro and in vivo.

As discussed further in the Examples, compositions of the invention (with or without the anti-fibrotic agent decorin) are able to inhibit myofibroblast differentiation in vivo, in an experimental model of microbial keratitis. The compositions, and particularly those incorporating decorin, are also able to maintain this reduced differentiation over time.

Myofibroblast differentiation may be increased in response to the action of TGF-β₁, a fibrotic growth factor that causes induction of α-sma expression. The Examples set out details of in vitro studies (in human dermal fibroblasts), which illustrate the ability of compositions of the invention to block this increase in α-sma expression. This illustrates that the beneficial inhibition of scarring achieved by the compositions of the invention is not limited to the eye. Furthermore, the inhibition of scarring appears to be an anti-fibrotic effect of the gel compositions themselves, since it is observed even in the absence of active anti-fibrotic agents.

Fibrosis is also associated with the expression and deposition of ECM constituents. The amount of ECM deposited may be increased in scarring, and the arrangement of the ECM may be different from that found in undamaged comparator tissue. The data presented in the Examples illustrate that treatment using compositions of the invention gives rise to tissues in which the arrangement of ECM components more close resembles that of unwounded tissue, thus illustrating the utility of these compositions in the inhibition of scarring.

The compositions of the invention are suitable for use at sites of surgical incisions, to inhibit scarring that may otherwise be associated with the healing of such surgical wounds.

An anti-fibrotic agent suitable for incorporation in a composition of the invention may be able to achieve an inhibition of fibrosis of at least 5% as compared to a suitable control agent. For example, a suitable anti-fibrotic agent may be able to achieve an inhibition of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a suitable control agent. An anti-fibrotic agent suitable for incorporation in a composition of the invention may be able to achieve substantially total inhibition of scarring as compared to a suitable control agent.

By the same token, the medical use of compositions of the invention, or methods of treatment using such compositions, to inhibit scarring may achieve an inhibition of at least 5% as compared to a suitable control. For example, such medical uses or methods of treatment may achieve an inhibition of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, as compared to a suitable control. The medical uses or methods of treatment of the invention may achieve substantially total inhibition of scarring as compared to a suitable control.

The selection of a suitable control will be readily determined by one of skill in the art. Merely by way of example, a suitable control for assessment of the ability of a composition of the invention to inhibit scarring in the eye may be provided by the recognised standard of care, or an experimental proxy thereof.

Active Agents Suitable for Incorporation in the Compositions of the Invention

Compositions of the invention intended for medical use, or use in methods of treatment, may comprise a further active agent. A suitable active agent may be selected with reference to the intended medical use. However, for illustration, a suitable active agent may be selected from the group consisting of: an anti-fibrotic agent; an anti-infective agent; a pain relief agent; an anti-inflammatory agent; an anti-proliferative agent; a keratolytic agent; an extracellular matrix modifying agent; a cell junction modifying agent; a basement membrane modifying agent; a biological lubricating agent; and a pigmentation modifying agent. For the avoidance of doubt, a composition of the invention may suitable comprise more than one active agent. In cases where the composition comprises more than one active agent, this may be more than one active agent within a particular class of active agents (e.g. two or more anti-fibrotic agents), or a combination of agents selected from two or more different classes (e.g. an anti-fibrotic agent and an anti-infective agent, or an anti-fibrotic agent and a pain relief agent).

Examples of anti-fibrotic agents that may be incorporated in compositions of the invention are discussed in more detail below.

Merely by way of example, an anti-infective agent suitable for incorporation as an active agent in a composition of the invention may be an anti-microbial agent, an anti-viral agent, an anti-fungal agent, or anti-helminth agent. In the case of an anti-microbial agent, a suitable anti-infective agent may be an antibiotic, such as gentamicin, penicillin, streptomycin (optionally in combination, as penicillin-streptomycin), or vancomycin. Many other suitable examples of antimicrobial agents that can be incorporated in compositions of the invention, including further antibiotics, will be well known to those skilled in the art.

A composition of the invention comprising an anti-infective agent may be used in methods for the prevention and/or treatment of infection. Accordingly, it will be appreciated that such a composition may be administered to a subject in need of prevention and/or treatment of infection. A subject in need of such prevention and/or treatment may be one that has a chronic wound or an infected wound. Merely by way of example, a subject at risk of developing a chronic wound may be one that has diabetes mellitus, chronic venous insufficiency, or peripheral arterial occlusive disease.

Embodiments of the compositions or methods of the invention employing anti-infective agents may also be useful in the prevention or treatment of disorders such as scarring that may associated with an infection (such as microbial keratitis).

A pain relief agent suitable for incorporation as an active agent in a composition of the invention may be selected from the group consisting of: an analgesic, an anaesthetic, such as benzocaine, proparacaine, tetracaine, articaine, dibucaine, lidocaine, prilocaine, pramoxine and dyclonine, or an ester, amide or ether thereof; a salicylate, such as salicylic acid or acetylsalicylic acid; a rubefacient, such as menthol, capsaicin and/or camphor, and a non-steroidal anti-inflammatory drug (NSAID), such as ibuprofen.

A composition of the invention comprising a pain relief agent may be used in methods for the prevention and/or treatment of pain. Accordingly, such a composition may be administered to a subject in need of prevention and/or treatment of pain. Suitably, a subject in need of such prevention and/or treatment may be one who has or is at risk of a condition that is associated with dermal or musculoskeletal pain.

An anti-inflammatory agent for incorporation as an active agent in a composition of the invention may be selected from the group consisting of: a steroid, such as a corticosteroid (for example prednisolone or dexamethasone); an NSAID, such as ibuprofen, or a COX-1 and/or COX-2 enzyme inhibitor; an anti-histamine, such as an H1 receptor antagonist; interleukin-10; pirfenidone; an immunomodulatory agent; and a heparin-like agent. Dextrans, or modified dextran sulphates, and decorin also represent suitable agents that may be incorporated in the compositions of the invention as anti-inflammatory agents. The skilled person will appreciate that these molecules are able to exert either anti-inflammatory or pro-inflammatory effects in vivo, but will be aware that the scientific and clinical literature provides a wealth of information to allow the selection of an appropriate dose to exert desired activity (be that anti-inflammatory or pro-inflammatory).

A composition of the invention comprising an anti-inflammatory agent may be used in methods for the prevention and/or treatment of inflammation. Accordingly, such a composition may be administered to a subject in need of prevention and/or treatment of inflammation. Suitably, the subject may be one having or at risk of developing chronic inflammation or acute inflammation. Merely by way of example, chronic inflammation may be associated with rheumatoid arthritis or dermatitis. Acute inflammation may be due to a wound.

An anti-proliferative agent for incorporation as an active agent in a composition of the invention may be selected from the group consisting of: a toll-like receptor 7 (TLR7) agonist, a toll-like receptor 2 (TLR2) agonist, a toll-like receptor 4 (TLR4) agonist, a toll-like receptor 9 (TLR9) agonist; and an antimetabolite. A suitable example of such a TLR7 agonist is imiquimod. A suitable example of such an antimetabolite is fluorouracil (5-FU).

A composition of the invention comprising an anti-proliferative agent may be used in methods for the prevention and/or treatment of a proliferative disorder. Accordingly, such a composition may be administered to a subject in need of prevention and/or treatment a proliferative disorder. Suitably, the subject may be one who has or is at risk of developing a skin proliferative disorder, such as psoriasis, cancer (for example melanoma or non-melanoma skin cancer), eczema, or ichthyosis.

A keratolytic agent for incorporation as an active agent in a composition of the invention may be selected from the group consisting of: an acid, such as salicylic acid, alpha hydroxy acid, beta hydroxy acid and/or lactic acid; an enzyme, such as papain and/or bromelain; a retinoid, such as retinol and/or tretinoin. Compositions or methods of the invention employing keratolytic agents (such as bromelain) may be used in the debridement of wounds, such as burns.

An extracellular matrix modifying agent suitable for incorporation in a composition of the invention may be selected from the group consisting of: proteinases (such a proteinase K), matrix metalloproteinases (MMPs); Membrane Type MMPs (MTMMPs); adamalysins (ADAMs); ADAMs with a thrombolysin (ADAMTS); disintegrins; tissue inhibitor of metalloproteinases (TIMPs); serine proteases such as urokinase; tissue plasminogen activator; elastase; matriptase; and enzymes such as cathepsins, heparanases and sulphatases implicated in matrix remodelling processes.

Compositions or methods of the invention employing extracellular matrix modifying agent may be used in applications that require modulation and remodelling of the ECM and/or modulation of cell-cell adhesion and cell-matrix interactions. By way of example, such applications may include the treatment of hypertrophic or keloid scars. Compositions or methods in accordance with such embodiments may provide clinical advantages by promoting the beneficial balances of collagen ratios or by directly targeting the production of ECM constituents such as collagen.

A cell junction modifying agent suitable for incorporation in a composition of the invention may be selected from the group consisting of: adenosine triphosphate (ATP); cyclic adenosine monophosphate (cAMP); inositol triphosphate (IP3); glucose; glutathione; glutamate; and ions selected from sodium, potassium and calcium ions. Suitably, such a cell junction modifying agent may be an antibody or other peptide that affects components of the cell junction, such as the connexins. Examples of such proteins include cadherins and α- and β-catenin. Suitably such an agent may achieve microtubular interference. Tight junctions might be affected by interference with components such as occludin, claudin(s) and junctional adhesion molecule-1 (JAM-1).

Platelet rich plasma (serum) may be incorporated in a composition of the present invention.

Compositions or methods of the invention employing cell junction modifying agents may be used in the treatment of chronic wounds, such as ulcers, that are hard-to-heal.

A basement membrane modifying agent suitable for incorporation in a composition of the invention may be an agent directed against adhesion. Such an agent may be selected from the group consisting of blocking antibody or competing peptides that inhibit the activity of integrins, laminins or components of Focal Adhesions (such as vinculin, talin, α-actinin, kindlin etc.). Alternatively a suitable basement membrane modifying agent may comprise a proteinase, such as proteinase K.

Compositions or methods of the invention employing basement membrane modifying agents may also be used in the treatment of chronic wounds, such as ulcers, that are hard-to-heal.

A biological lubricating agent is, for the purposes of the present disclosure, to be taken as being an agent, derived from a biological source, that is capable of serving as a lubricant. In a suitable embodiment, a biological lubricant for incorporation in a hydrogel composition of the invention may be serum. As set out below, serum has therapeutic utility in the treatment of a number of disorders of the eye. Accordingly, a hydrogel composition of the invention comprising serum may be suitable for ocular administration, as eye drops.

Compositions or methods of the invention employing a biological lubricating agent, such as serum, may be used in the prevention and/or treatment of conditions including those selected from the group consisting of: dry eye syndrome; and Sjögren's syndrome.

Compositions or methods of the invention may employ a pigment modifying agent. A pigment modifying agent for incorporation as an active agent in a composition of the invention may be selected from the group consisting of: a depigmenting agent; and a pigmentation promoting agent.

Suitable depigmenting agents for incorporation in a composition of the invention may be selected from the group consisting of turmeric; a melanin production inhibitor; and an antioxidant. A suitable example of a melanin production inhibitor may include hydroquinone, resorcinol, resveratrol, or azelaic acid. A suitable example of an antioxidant may include vitamin C, vitamin E, glutathione, turmeric, or ferulic acid.

Pigmentation promoting agents suitable for incorporation in a composition of the invention include substances that affect components of the melanin pathway. These may be selected from the group consisting of: tyrosine (which is hydroxylated to L-3,4-dihydroxphenylalanine (DOPA) by tyrosinase); and DOPA (which is oxidised to DOPAquinone and, in the presence of a cysteine group, phaeomelanin is produced). Eumelanin production requires the actions of two further enzymes: tyrosinase-related protein 1 (TRP1) and 2 (TRP2/Dct) which rearrange DOPAchrome (produced from the spontaneous cyclic oxidation of DOPAquinone) to form DHI-2-carboxylic acid (DHICA). These enzymes or their substrates may also represent suitable pigmentation modifying agents.

Compositions or methods of the invention employing pigmentation modifying agents may be used in a wide range of clinical contexts associated with undesirable hypo or hyper pigmentation. These include scarring, such as following surgery or pathological scarring (such as hypertrophic or keloid scarring).

A composition of the invention comprising a depigmenting agent may be used in methods for the prevention and/or treatment of a hyperpigmentation disorder. Accordingly, such a composition may be administered to a subject in need of prevention and/or treatment a hyperpigmentation disorder. Suitably, the subject may be one who has or is at risk of melasma, post inflammatory hyperpigmentation, or Addison's disease.

In a suitable embodiment, a composition in accordance with the invention may comprise an anti-fibrotic agent for use in combination with one or more agents selected from the group consisting of: a steroid; and an antimicrobial agent. The anti-fibrotic agent, steroid and antimicrobial agent may be formulated in separate compositions, or as part of the same composition.

Suitably a composition of the invention may comprise decorin for use in combination with the anti-infective agent gentamicin, and the anti-inflammatory agent prednisolone. A composition of this sort may comprise decorin, prednisolone and gentamicin. Such compositions of the invention are suitable for use in the inhibition of scarring associated with microbial keratitis, as illustrated by the data set out in the Examples.

In a suitable embodiment, a composition of the invention may comprise an anti-inflammatory agent and a pain relief agent. Such a composition may be of particular utility in the context of, for example, a chronic inflammatory disease such as dermatitis or rheumatoid arthritis, where it may be desirable to prevent and/or treat pain and inflammation.

In another example, the composition of the invention may comprise a pain relief agent and an anti-infective agent. Such a composition may be of particular utility in the context of a dermal wound, where it may be desirable to prevent and/or treat pain and infection. Other suitable combinations of active agents will be known to those skilled in the art.

A composition of the invention for medical use may incorporate an active agent in a therapeutically effective amount. Such a therapeutically effective amount will be able to achieve a desired clinical outcome either in a single administration, or as part of a course of treatment comprising multiple incidences of administration. The skilled person will be well aware of suitable protocols and procedures for the calculation of therapeutically effective amounts of active agents of various sorts.

Suitably an active agent may be incorporated in a composition of the invention at a concentration of between 0.1 ng/mL and 10 mg/mL. For example, an active agent may be incorporated in a composition of the invention at a concentration of between 1 ng/mL and 5 mg/mL, between 10 ng/mL and 2.5 mg/mL, or between 20 ng/mL and 1 mg/mL, between about 0.1 μg/mL and 0.5 μg/mL, suitably about 0.24 μg/mL.

Anti-Fibrotic Agents

Anti-fibrotic agents are agents that are able to bring about an inhibition of scarring in a subject, or body site, to which they are provided. The inhibition of scarring is considered more generally below.

Many anti-fibrotic agents are known to those skilled in the art. Accordingly, the skilled person will be readily able to identify anti-fibrotic agents that may beneficially be incorporated in compositions of the invention that are for use in the inhibition of scarring. The following provides a non-exclusive list of examples of anti-fibrotic agents suitable for such uses.

Suitable anti-fibrotic agents may be selected from the group consisting of: anti-fibrotic extracellular matrix (ECM) components; anti-fibrotic growth factors (which for purposes of the present disclosure should be taken as also encompassing anti-fibrotic cytokines, chemokines, and the like); polymers such as dextrans or modified dextran sulphates; and inhibitors of fibrotic agents, such as function blocking antibodies. It will be appreciated that the therapeutic effectiveness of such agents will depend upon the doses provided by the compositions of the invention. The skilled person will be aware of a wide range of literature and clinical resources to allow the selection of a suitable dose of any of the listed agents to fulfil a required therapeutic aim.

Dextrans, or modified dextran sulphates, are able to exert both anti-fibrotic and pro-fibrotic effects in vivo. In the context of anti-fibrotic use of dextrans or modified dextran sulphates, the skilled person will appreciate that suitable doses for anti-fibrotic purposes may be between 0.1 and 10 mg/kg bodyweight of the subject. In a suitable embodiment a dextran, or modified dextran sulphate, for use in a composition of the invention may have a molecular weight of 10 kDa or less.

Antibodies are useful in disrupting certain cellular activities by binding to cell signalling agents and thereby blocking functions caused by the agents' activity. Examples of such activities that may be blocked include: cell proliferation, cell migration, protease production, apoptosis and anoikis. Merely by way of example, suitable blocking antibodies may be able to bind one or more of the following groups of cell signalling agents: ECM components, growth factors, cytokines, chemokines or matrikines. Decorin is an example of an anti-fibrotic ECM component that may advantageously be incorporated in the compositions of the invention. The decorin may be human decorin.

Suitably the decorin may be human recombinant decorin. An example of a human recombinant decorin that may be incorporated in the compositions of the invention is that produced and sold by Catalent Pharma Solutions, Inc., under the name Galacorin™.

Decorin for incorporation in a composition of the invention may be a full-length naturally occurring version of this proteoglycan. Alternatively, compositions of the invention may employ anti-fibrotic fragments or anti-fibrotic variants of naturally occurring decorin.

Naturally occurring decorin is a proteoglycan. The proteoglycan (comprising both the core protein and glycosaminoglycan chains), or its fragments, may be used in the hydrogel compositions of the invention. However, the inventors have demonstrated that the core protein alone (without glycosaminoglycan chains) is sufficient to inhibit scarring in the eye. Accordingly, references to decorin (or fragments or variants thereof), in the present specification may alternatively be construed as directed to the core protein without glycosaminoglycan chains. The inventors believe that it is the core protein of decorin that serves to bind to fibrotic growth factors (such as TGF-β), and to block their biological function.

A suitable anti-fibrotic fragment of decorin may comprise up to 50% of the full-length, naturally occurring molecule, up to 75% of the full-length, naturally occurring molecule, or up to 90% of the full-length, naturally occurring molecule. A suitable anti-fibrotic fragment of decorin may comprise the TGF-β-binding portion of decorin.

An anti-fibrotic variant of decorin will differ from the naturally occurring proteoglycan by the presence of one or more mutations in the amino acid sequence of the core protein. These mutations may give rise to additions, deletions, or substitutions of one or more amino acid residues present in the core protein. Merely by way of example, a suitable anti-fibrotic variant of decorin suitable for incorporation in the compositions of the invention may comprise at least 1, at least 2, at least 3, at least 4, at least 5, at least 10, at least 15, or at least 20 mutations as compared to the amino acid sequence of the naturally occurring core protein.

Except for where the context requires otherwise, references herein to decorin, in connection with the incorporation of this agent in the compositions of the invention, should also be taken as encompassing the use of anti-fibrotic fragments or anti-fibrotic variants of decorin.

In a suitable embodiment, decorin constitutes the only ECM component present in a composition of the invention.

Anti-fibrotic growth factors suitable for incorporation in compositions of the invention include those selected from the group consisting of: transforming growth factor-β3, platelet derived growth factor AA, insulin-like growth factor-1, epidermal growth factor, fibroblast growth factors (FGF) 2, FGF7, FGF10, FGF22, vascular endothelial growth factor A, keratinocyte growth factor, and hepatocyte growth factor.

Inhibitors of fibrotic agents represent suitable anti-fibrotic agents that may be incorporated in the compositions of the invention. Examples of such inhibitors include agents that bind to, and thereby block, the activity of a fibrotic agent. Examples of such inhibitors include function blocking antibodies (discussed further above), or soluble fragments of cell receptors by which the fibrotic agent induces cell signalling. Other examples of such inhibitors include agents that prevent expression of the fibrotic agent. Examples of these sorts of inhibitors include those selected from a group consisting of: anti-sense oligonucleotides, and interfering RNA sequences.

A composition of the invention suitable for use in the inhibition of scarring may incorporate an anti-fibrotic agent in a therapeutically effective amount. Such a therapeutically effective amount will be able to inhibit scarring either in a single administration, or as part of a course of treatment comprising multiple incidences of administration. Details of how inhibition of scarring may be assessed, and so how a therapeutically effective amount may be calculated or recognised, are considered above.

Merely by way of example, an anti-fibrotic agent, such as decorin, may be incorporated in a composition of the invention at a concentration of between 0.1 ng/mL and 10 mg/mL, between 1 ng/mL and 5 mg/mL, between 10 ng/mL and 2.5 mg/mL, between 20 ng/mL and 1 mg/mL, between about 0.1 μg/mL and 0.5 μg/mL, suitably about 0.24 μg/mL.

Topical Administration and Topical Compositions

The compositions of the invention are suitable for topical administration to a subject. For the avoidance of doubt, in the context of the present disclosure, “topical administration” is taken to relate to direct administration of the composition to a surface of the body or a surface of an organ. A composition of the invention suitable for such topical administration may be referred to as a topical composition of the invention.

Suitably topical compositions of the invention may be for administration to one or more body surfaces selected from the group consisting of: a surface of the eye; the skin; a surface of the brain; and a mucous membrane. By way of example, the topical compositions of the invention may be administered to a body surface during or after surgery. Suitably the topical compositions of the invention may be administered to such a surface in association with abdominal surgery (e.g. to inhibit adhesion formation), or brain surgery (e.g. to provide a desired therapeutic agent to the brain).

Topical compositions of the invention may be for administration to sites of infection or injury (including, but not limited to: abrasions, burns, and puncture wounds) on a body surface. For example, a composition of the invention may be for administration to a site of infection or injury on the surface of the eye (such as a site of microbial keratitis), or a site of infection of or injury to the skin (such as a skin burn or abrasion).

It will be appreciated that topical compositions may be formulated in manners conventional for use in such contexts. For example, a suitable topical composition may be formulated such that it does not induce irritation or inflammation of an infected or injured area to which it is administered.

The inventors have provided a novel eye drop system for the sustained delivery of a potent anti-scarring molecule (hrDecorin). The novelty of this eye drop lies in the method of structuring during manufacture, which creates a material that can transition between solid and liquid states, allowing retention in a dynamic environment being slowly removed through blinking. In a murine model of Pseudomonas keratitis, applying the eye drop resulted in reductions of corneal opacity within 16 days. More remarkably, the addition of hrDecorin resulted in scarless restoration and corneal integrity, as shown by complete re-epithelialization and reductions in αSMA, fibronectin and laminin. This drug delivery system is an ideal non-invasive anti-fibrotic treatment for patients with microbial keratitis, potentially without recourse to surgery saving the sight of many in the developing world, where corneal transplantation may not be available.

The present inventors have provided report a new class of eye drop material that allows for prolonged retention of a therapeutic on the surface of the eye, while being gradually cleared through the blinking process. The material is formed through the shearing of a gellan-based hydrogel, a material that is currently used in dilute form to thicken eye drops (e.g. Timoptol) during the gelation process. The application of shear prevents the formation of a continuous polymeric network and results in the formation of interacting particles that can exhibit spherical and ribbon-like morphology. Following shear-processing, these particles interact and form a continuous structure when the solution is at rest. When shear is applied (such as when extruded through an eye dropper), however, the continuous network of particles is disturbed and the material liquefies. Subsequent removal of the shear force results in an immediate healing. The solid-liquid-solid transitions that this material is able to undergo means that it conforms perfectly to the ocular surface and is removed gradually by the eyelid blinking dynamics. Importantly, gellan gum is optically transparent and so the material can continue to transmit light following application, causing minimal disruption to the patient.

A fluid-gel eye drop has been developed which can be loaded with decorin, to provide localized drug delivery and retention at the surface of the eye. The material combines structured gellan gum with the proteoglycan, decorin. Additionally, in conjunction to high optical clarity, the FDA approved polymer (FDA reference number 172.665) coupled with clinical grade hrDecorin, provides a rapid route to translation into the clinic. As such, this study investigated the effects of fluid gel, with and without hrDecorin, on corneal opacity, wound healing and fibrosis within a well-established murine model of Pseudomonas keratitis, as a precursor to clinical application for the management of severe bacterial infection.

The inventors have demonstrated that fluid-gel eye drops as described herein are beneficial in the prevention and/or treatment of glaucoma. Fluid-gel eye drops for use in accordance with the invention, and in particular in accordance with this aspect of the invention, may comprise shear-thinning hydrogel compositions comprising gellan. The inventors have found, as demonstrated in the results disclosed elsewhere in the present specification, that shear-thinning hydrogel compositions in accordance with the invention are able to reduce intraocular pressure (a well known experimental model for glaucoma) even when formulated without active agents.

### Fluid Gel Formulation and Properties

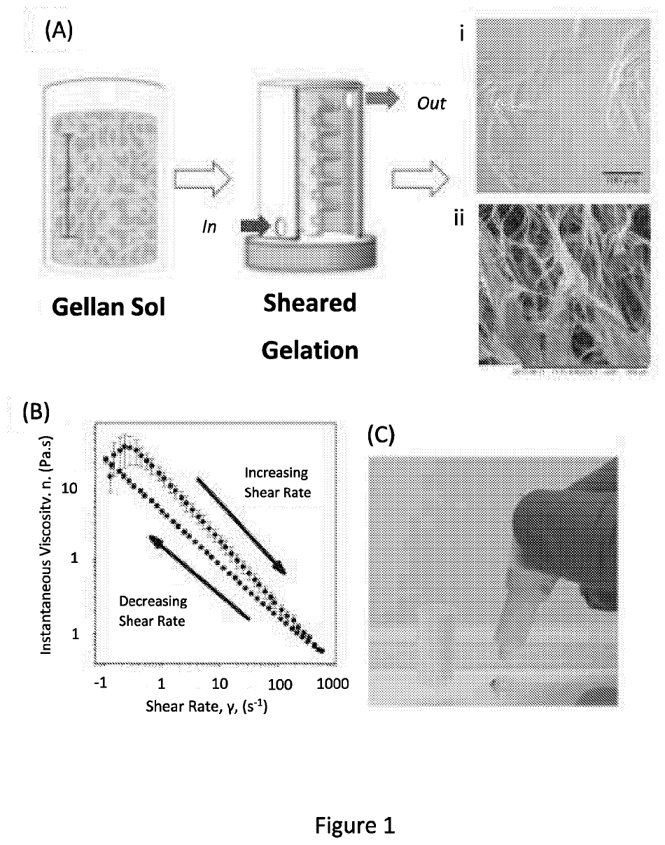

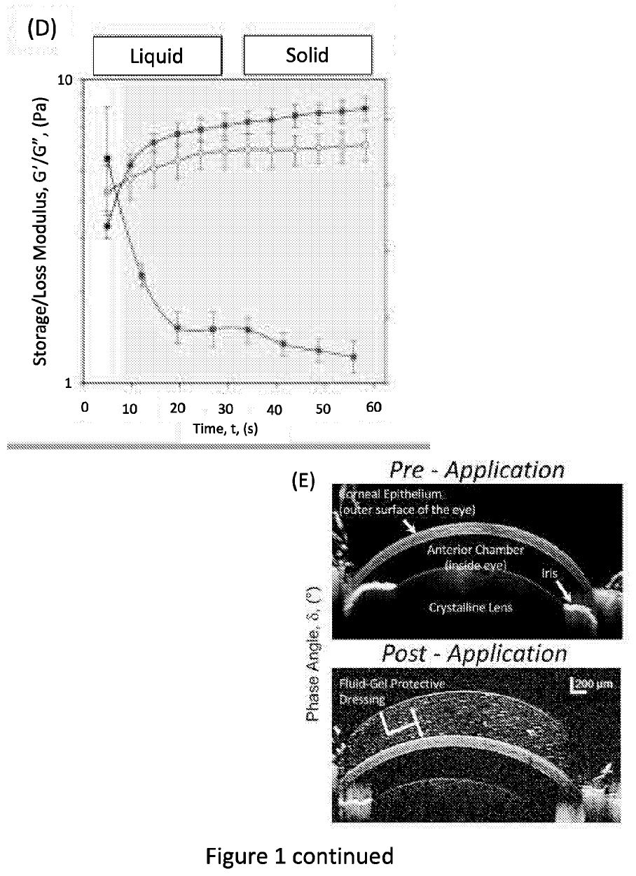

Processing of the fluid gel involves passing a polymer solution, gellan, through a jacketed pin-stirrer, where it experiences high levels of shear whilst being forced (thermally) through its sol-gel transition (FIG. 1a). This restricts the long-range ordering normally observed in the formation of quiescent gels, restricting growth of the gel nuclei to discrete particles^{[34, 35]}. The microstructures within the eye drop prepared in this way have been shown using two techniques: 1) optical microscopy, whereby the refractive index of the continuous phase was manipulated using polyethylene glycol, and 2) lyophilizing in order to image using scanning electron microscopy (SEM) (FIGS. 1a(i) and 1a(ii), respectively). Both microscopic techniques highlight the stranded microstructure of the resulting gelled entities, where their large length to width ratio and subsequent large hydrodynamic radii, give rise to the resulting material properties (viscosity and elastic structuring)^{[36]}.

The unique properties of fluid gels are such that they exhibit pseudo-solid properties at rest, but can be made to flow under force. Here, increasing the shear force exerted on the system, results in non-Newtonian, shear thinning behavior, typical of highly flocculated or concentrated polymer dispersions/solutions^{[37]} (FIG. 1b). As such, at low shear, large viscosities exceeding several orders of magnitude higher than typical water based eye drops are observed, thinning during application and subsequent blinking as a result of dis-entanglement and alignment of particles in flow^{[38, 39]}. This makes the microgel suspension ideal for application through dropper bottles, rapidly shear thinning through the nozzle on application to the eye (FIG. 1c). Post-application, restoration of the 3-dimensional structural matrix is key to gaining high retention times upon the ocular surface. Time dependent removal of shear, at comparative timescales to the initial ramp, was used to gather information regarding such structuring, probing eye drop hysteresis. The eye drop system showed a degree of thixotropy (FIG. 1b), whereby the majority of the original viscosity was recovered. The presence of weak interactions between gel-ribbons were examined using linear rheology, with the evolution of an elastic structure at strains within the linear viscoelastic region (FIG. 1d). It was observed that initially, post-shear, the fluid gel exhibited typical liquid-like behavior with the loss modulus (G″) dominating the storage modulus (G′). Following this, an increase in G′ as a function of the formation of interactions between gelled ribbons led to a crossover being reached, at which point the system began to behave as a solid-gel^{[40]}. Further structuring over time thus leads to pseudo-solid behaviors, where a continuous network is formed between the gelled entities. The ability to shear thin on application, whilst being able to quickly restructure post-shearing, enables the eye drop to be applied to the ocular surface, acting as a barrier. Using a single 5 μl application of fluid gel eye drop, it was demonstrated that a uniform distribution of gel covers the entire ocular surface including cornea, adjacent conjunctiva and fornices (space between the eyelid and eyeball) in a rodent eye (FIG. 1e).

### In Vitro Eye Drop Activity

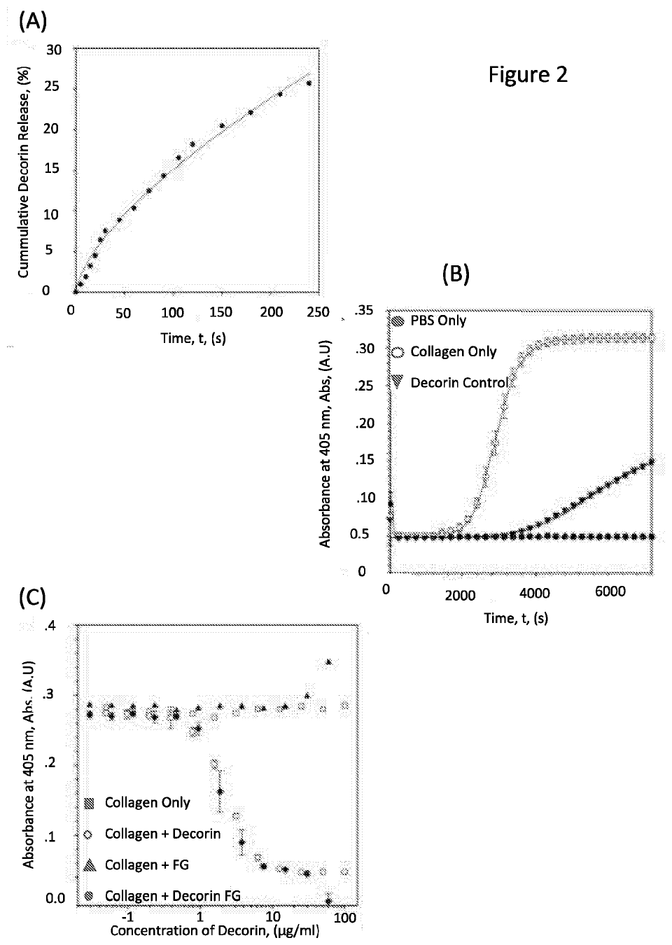

The gellan-based eye drop system was formulated for drug delivery with the candidate anti-fibrotic agent, hrDecorin, used for our studies. The rate of release of hrDecorin from the eye drop system was almost linear over time (FIG. 2a). Turbidity was used as a measurement of fibrillogenesis (formation of large, disorientated collagen fibers), shown as a function of the hrDecorin (FIGS. 2b&c). It was evident that hrDecorin played a key role in the kinetics of fibril formation, slowing the onset of fibrillogenesis, and also reaching an equilibrium much faster (FIG. 2b). Above a critical concentration, 0.5 μg/ml, an active effect of hrDecorin in inhibiting fibrillogenesis was observed, highlighting a concentration dependency until a minimum turbidity is achieved (>10 μg/ml), above which no further reduction occurs (FIG. 2c). Furthermore, the assay demonstrated that the fluid gel carrier had no effect on fibril formation, correlating closely with the collagen only controls.

### In Vivo Efficacy of the Loaded/Unloaded Eye Drop on Corneal Opacity

Using a well-established model of bacterial keratitis^{[41]}, anaesthetized mice (n=6 per group) were challenged with P. aeruginosa (10⁵ CFU) upon the surface of the damaged cornea. A therapeutic protocol to treat the infection based upon the standard treatment for bacterial keratitis patients was developed. After 12 hours of P. aeruginosa incubation to establish the corneal infection, eyes were treated with a 2-hourly regime of Gentamicin (1.5%) over a 12-hour period to sterilize the infection (confirmed by swab cultures).

Following the sterilization phase, 2 days after the initial inoculation, single 5 μl gellan eye drops were administered every 4 hours between 8 am and 8 μm for a further 13 days covering the treatment groups of 1) Gentamicin and Prednisolone (G.P); 2) Gentamicin, Prednisolone s and fluid gel (G.P.FG) and; 3) Gentamicin, Prednisolone and hrDecorin fluid gel (G.P.DecFG) (Table 1).

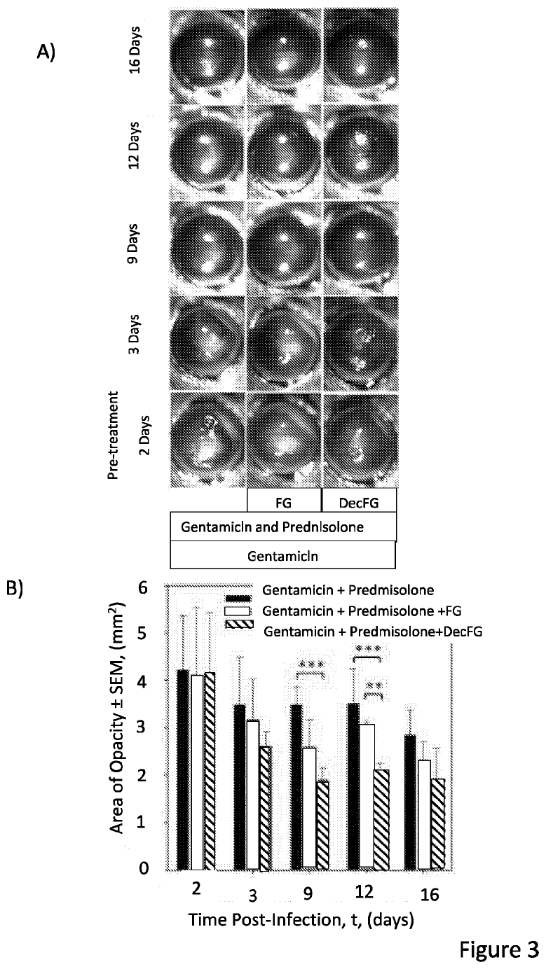

Images of the cornea were taken at intervals throughout the 16-day experiment to measure changes in corneal opacity (FIG. 3a). All mice were euthanized on day 16. The area of opacity (measured independently by two clinical ophthalmologists, masked to treatment groups) showed earlier size-reduction in eyes treated with the fluid gel and with the hrDecorin fluid gel eye drops plus standard of care compared to eyes treated with standard of care (Gentamicin and Prednisolone) treatments alone. Accordingly, at day 9, eyes treated with the standard of care with hrDecorin fluid gel, showed significantly (p<0.001) lower opaque areas (1.9±0.3 mm²) compared with eyes treated with Gentamicin and Prednisolone only (3.5±0.4 mm²). At 12 days, the mice that received hrDecorin fluid gel eye drops with standard of care maintained significantly lower (p<0.01) opaque area compared with the Gentamicin and Prednisolone group, and also to the fluid gel group with standard of care (mean opacity area in Group 1=3.5±0.7 mm², Group 2=3.0±0.1 mm², in comparison to Group 3=2.1±0.2 mm²; FIG. 3b).

Effects of the Fluid Gel Eye Drops with and without hrDecorin on Corneal Re-Epithelialization

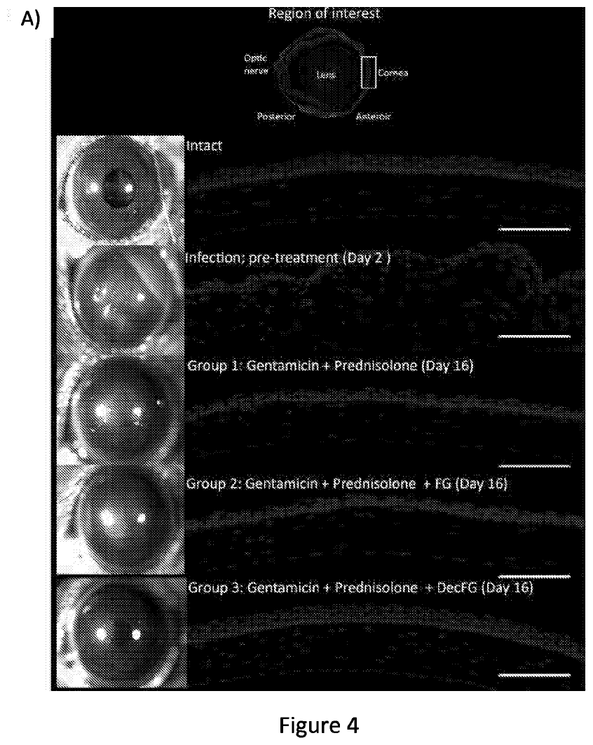

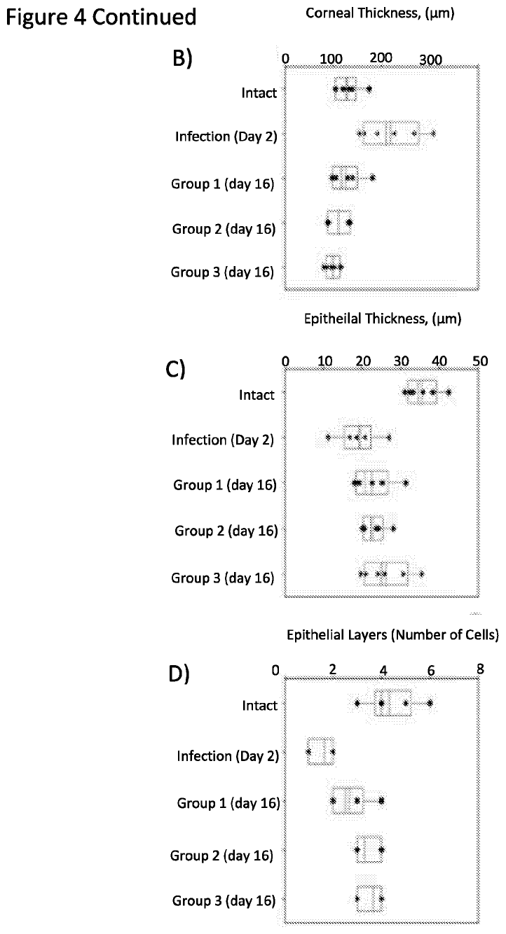

Epithelial stratification/maturation together with stromal thickness were chosen as outcome measures to assess corneal re-epithelialization, and to observe thickening of the stroma from edema and cellular infiltrates (as markers of infection). Pseudomonas infection severely disrupted the corneal structure, with an averaged increased corneal thickness of 218.7±24 μm after the infection on day 2 compared to naïve corneal thickness values of 129.3±10.7 μm. The infected corneas at day 2 had thinner epithelial layers compared to normal intact controls (19.2±2.1 μm vs 35.5±1.7 μm; FIGS. 4a & b). With the addition of fluid gel alone and with hrDecorin eye drops treatments over 13 days it was evident that re-epithelialization was improved. Treatment with the hrDecorin loaded fluid gel eye drop led to an epithelial layer with an improved degree of stratification (26.1±2.4 μm thick made up of 3.6±0.2 cell layers) compared to the epithelium in the Gentamicin and Prednisolone group (22.5±2.1 μm thick with 2.7±0.2 cell layers) as well as the Gentamicin, Prednisolone and fluid gel group (22.8±1.3 μm thick with 3.4±0.1 cell layers). However, the differences between the various groups did not reach statistical significance (FIGS. 4b, c and d).

### Effects of the Fluid Gel on Levels of Myofibroblasts and Extracellular Matrix

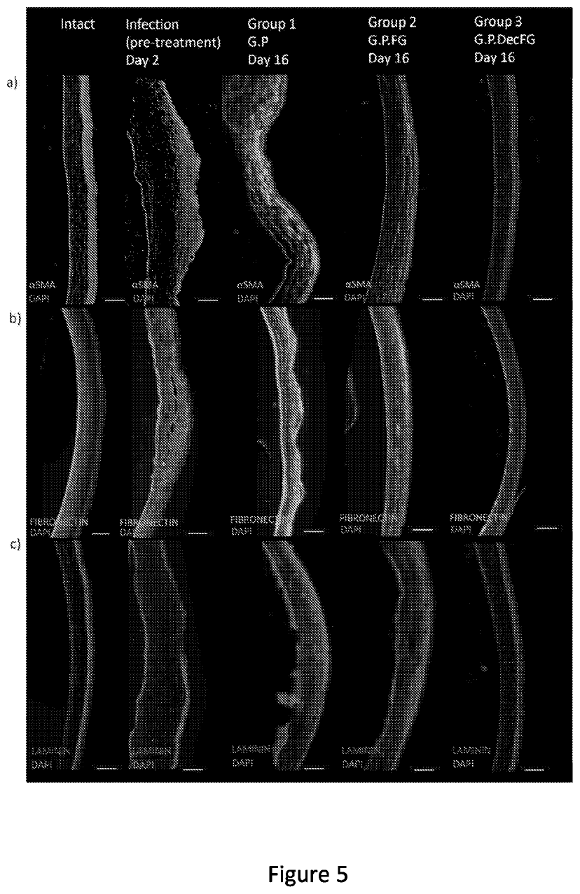

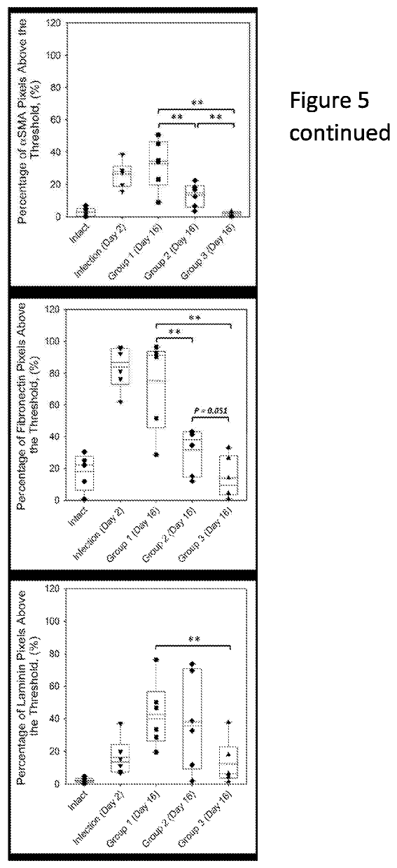

Immunoreactivity (IR) was used to assess the degree of fibrosis, as a ratio of pixel intensity above a baseline obtained from intact corneas (referred to here on as the threshold). In the naïve intact cornea, there were very low levels of αSMA immunoreactivity (IR) in the corneal stroma indicating the presence of few myofibroblasts (FIG. 5a). Two days after the infection, one day after sterilization, infected cornea demonstrated a 23% increase in stromal αSMA staining to levels of 26.5±3.0% above the threshold (normalized from an intact cornea), indicating increased differentiation of myofibroblasts. Levels of stromal IR αSMA remained elevated at day 16, at 32.7±6.1% in eyes treated with standard of care only. When eyes were also treated with the fluid gel eye drops, either with or without hrDecorin, the level of stromal αSMA IR was significantly lower at day 16, 13.4±2.9% and 2.0±0.4% respectively, suggesting less myofibroblast activation within the corneal stroma. The hrDecorin fluid gel was most effective at keeping the αSMA IR levels low, resulting in similar values to the intact cornea, suggesting that the addition of hrDecorin in the fluid gel had an added beneficial effect on myofibroblast differentiation vs the fluid gel alone (FIG. 5a).

Stromal ECM levels generated by the myofibroblasts were studied using fibronectin and laminin IR (FIGS. 5b and c). Increased amounts of stromal IR fibronectin were observed post-infection at day 2, remaining high at day 16 after Gentamicin and Prednisolone treatment (IR Fibronectin 83.9±5.5% and 75.3±11.5%, at day 0 and 16, respectively). Fluid gel with or without hrDecorin, significantly lowered levels of fibronectin IR to 31.6±5.8% and 13.9±5.3% respectively, demonstrating borderline significant differences (p=0.051) between the two eye drop treatment groups. Levels of IR laminin (FIG. 5c) demonstrated that the infection increased levels of laminin when compared with the intact cornea, from 2.15±0.6% in the intact to 16.3±4.6% in the infection group at day 2. Levels of IR laminin continued to rise by day 16 after Gentamicin and Prednisolone treatment to 42.5±8.2%. Similar to the Gentamicin and Prednisolone group, average levels of IR laminin remained high on day 16 after treatment with the fluid gel, with IR laminin levels at 38.0±12.0%. The addition of hrDecorin to the fluid gel significantly lowered laminin levels compared to Gentamicin and Prednisolone treatment, (12.4±5.5% vs 42.3±8.2%), whilst the fluid gel without hrDecorin had no effect on this ECM parameter.

### Effects of the Fluid Gel on Levels of Myofibroblasts In Vitro

Human dermal fibroblast cells were grown at a density of 150,000 cells/well in 6 well plates. Cells were allowed to attach for 24 hours, before being serum starved in HFDM-1 medium prior to treatment with experimental compositions.

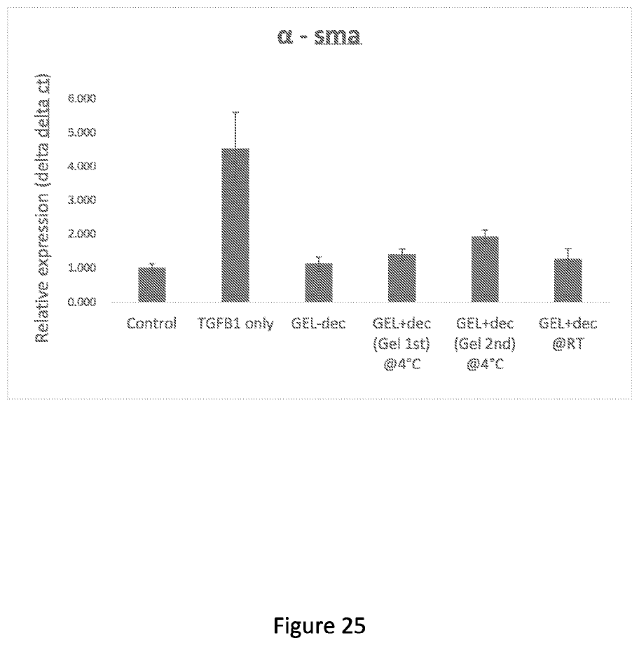

Experimental hydrogel compositions of the invention were prepared with or without the anti-fibrotic agent decorin. These are respectively shown as “GEL+dec” and “GEL-dec” in the graph of FIG. 25. In the study 1 ml of the experimental gel composition was added per well before dosing with TGF-β1 at 5 ng/ml (the exception being “GEL+dec(Gel 2nd)”, where TGF-β1 was provided prior to administration of the gel, demonstrating that the order did not markedly change the effect).

As can be seen from FIG. 25, addition of TGF-β1 stimulated expression of α-sma, indicative of formation of myofibroblasts characteristic of scarring. Provision of a hydrogel composition of the invention reduced this expression of α-sma. This was observed in both the presence and absence of the anti-fibrotic agent decorin, illustrating the ability of the hydrogel compositions of the invention to inhibit scarring, even in the absence of further anti-fibrotic active agents.

### Discussion

Improving ocular retention is key to increasing both bio-efficiency and therapeutic response to topical therapies, as turnover of the pre-corneal tear film (ca. 20% per minute^{[42]}) results in rapid elimination of aqueous drugs, reducing titers delivered to the target tissue site. As such, many ocular conditions are currently treated via intensive topical therapies delivered through the day and night, or invasive methods disliked by many patients, including periocular or intravitreal injections to target intraocular pathology. In more severe cases where drugs are ineffective, surgery may be required to treat or remove the resultant corneal scar, increasing the risk of morbidity and increasing the duration of patient discomfort following treatment. The structured or “fluid-gel” formed from gellan provides a pivotal advance since it enables the sustained delivery of molecules such as hrDecorin capable of preventing scarring and obviating the need for invasive surgical repair strategies. The major advantage of the gellan fluid-gel is its capacity to transition between solid and liquid states as it passes through the applicator and solidifies on the surface of the cornea. This unique set of properties originates from the microstructure of the material, which consisted of ribbons and particles that weakly interact with one another at zero shear. These interactions are broken by the application of shear and reform following its removal. In this way, the material may then be gradually cleared from the ocular surface through the natural blinking mechanism. The development of a weak-elastic structure when applied to the surface of the cornea, results in the formation of a transparent and resorbable bandage, with the benefits of eye drops (in application) and hydrogel lens (sustained release), without the drawbacks of either. Indeed, the fluid-gel alone seems to provide a micro-environment conducive to wound healing, with a reduction in corneal opacity and markers of scar formation even without decorin addition. Importantly, the fluid gel does not interfere with hrDecorin's biological activity, as shown by the collagen fibrillogenesis data. As such, the system provides an excellent candidate technology for the clinical setting, with improved drug administration compliance across numerous patient cohorts.

The mouse model of P. aeruginosa keratitis provides a robust, clinically relevant means of evaluating the anti-scarring capacity of the hrDecorin loaded fluid gel against the current standard of care for pseudomonas infection (Gentamicin and Prednisolone)^{[43]}. Once the infection is established, P. aeruginosa invades the corneal epithelial cells disrupting the natural healing responses with transformation of corneal fibroblast into corneal myofibroblasts leading to a fibrogenic microenvironment^{[44]}. Topical administration of the eye drops either with or without the hrDecorin resulted in reduced levels of corneal opacity after 7 and 10 days of eye drop treatments, with the addition of hrDecorin displaying an evident further advantage. The effects of the fluid gel only treatment were not expected as the initial in vitro studies demonstrated that this carrier appeared inert. The therapeutic efficacy of fluid gel alone may be due to the formation of a permissive microenvironment in the damaged cornea, where the occlusive effect of the gel ribbons (that entwine to form a barrier around the wound) provide two key effects. Firstly, a therapeutic bandage preventing biomechanical trauma caused by blinking over the ulcerated eye and secondly, sequestering steroid and Gentamicin within its structure, enhancing retention of the therapeutic substances to the ocular surface, and thereby improving bioavailability similar to the prosthetic replacement of the ecosystem (PROSE™ device) but with the added advantage of being resorbable. Such reductions in corneal opacity would benefit patients in terms of preservation of sight^{[45]}.

An important aspect to the healing phase encompasses restoration of a stratified non-keratinized epithelium. Together with the tear film, an apical mucosa (composed of lipid, mucins and aqueous layers) provides nutrition and lubrication to the ocular surface and is fundamental to first-line of defense to the eye. hrDecorin treated eyes exhibited the most improved restoration to normal anatomy, with a reduction in stromal edema, thickness and extracellular matrix deposition, coupled with improved epithelial morphology. The reduction in fibrotic markers by hrDecorin has been previously demonstrated across numerous animal models; modulating a range of growth factors (e.g. VEGF, IGF-1, EGF, PDGF) and their receptors, in particular TGFβ signaling via SMAD 2 and 3 pathways, preventing differentiation of corneal fibroblasts. Additionally, it's regulation of matrix metalloproteinase (MMPs) and tissue inhibitors of metalloproteinase (TIMP) results in fibrolysis and attenuated scar formation^{[29, 46-48]}.

The intrinsic ability of hrDecorin to aid healing, and in particular reduce scarring, has been enhanced by introducing the fluid gel carrier, improving retention time on the ocular surface. The benefits of this fluid gel formulation have been clearly demonstrated in vivo, observed both physically, with a reduction in corneal opacity, and pharmacologically, relating to diminished fibrotic markers. However, due to legislative constraints the data generated within this study was limited to a 16 day time point. However, it would be of interest to examine later time points in future studies.

The effects of the fluid gel alone on the damaged corneal surface suggests an influence over the endogenous growth factors, an effect that is enhanced by the addition of hrDecorin. The fluid-gel may aid corneal healing through several mechanisms: firstly, the unique viscoelastic properties of the fluid gel acts as a liquid that self-structures upon the ocular surface to form a semi-solid occlusive therapeutic dressing for unperturbed healing to take place; secondly, helical domains formed during the gelation of the fluid gel may provide a mimetic scaffold for endogenous decorin to bind, sequestering key growth factors e.g. TGF□ and/or exogenously delivered hrDecorin; thirdly, the fluid gel matrix, comprising primarily of water (99.1%), creates a gradient driven diffusion of cytokines away from the wound site, again resulting in a restoration of the natural equilibria needed to prevent fibrosis.

In conclusion, the inventors have demonstrated that a novel eye drop technology can be used to provide sustained delivery of anti-fibrotic drugs like hrDecorin topically to the cornea in a clinically relevant murine model of fibrosis associated with bacterial keratitis.

The eye drop enabled the hrDecorin to remain in contact with the surface of the eye for long enough and at sufficient titers to significantly reduce corneal scarring. Furthermore, this study has demonstrated that the unloaded fluid gel also possesses healing effects in its own right, suggested to arise through its intrinsic material microstructure and subsequent properties. Not only do the material properties of the eye drop enhance anti-scarring drug retention times, but the user-friendly nature of the drops would be welcomed by patients, providing a simple treatment to prevent the scarring pathology that is prevalent after corneal infection. Having demonstrated successful reduction in corneal opacity and a reduction in markers commonly indicative of the scarring process, when compared to the current standard of care, this technology presents an ideal treatment option for patients with microbial keratitis, reducing the occurrence of visually significant corneal opacity and potentially eradicating the need for corrective surgical intervention. Given that transplant availability and the facilities for surgical intervention are often not available in the developing world, we believe that this technology could, in the future, help to save the sight of many patients.

Materials and Methods

Study Design

The aim of this study was to explore the use of a novel fluid gel to deliver decorin to the ocular surface in order to reduce corneal opacity and scarring post-bacterial keratitis. The study was split into three evaluation stages: (i) material properties relating to ease of eye drop application, (ii) in vitro assessment of bioactivity of the formulated hrDecorin and (iii) anti-scarring efficacy of the fluid gel with/without hrDecorin in vivo, using a mouse model of Pseudomonas keratitis (once the eyes were sterilized after infection) in comparison to the current standard of care. The sample size (n=6 per experimental group) was based on the resource equation as the effect size was unknown. All analyses were performed by observers masked to experimental groupings and mice were randomly assigned to both treatment and control groups.

### Materials

Fluid Gel (FG) and hrDecorin Fluid Gel (DecFG) Production

### Preparation of Fluid Gel Eye Drops

Fluid gels were produced by first dissolving low acyl gellan gum (Kelco gel CG LA, Azelis, UK) in deionized water. Gellan powder was added to deionized water at ambient temperature in the correct ratio to result in a 1% (w/v) solution. The sol was heated to 70° C. under agitation, on a hotplate equipped with a magnetic stirrer, until all the polymer had dissolved. Once dissolved, gellan sol was added to the cup of a rotational rheometer (AR-G2, TA Instruments, UK) equipped with cup and vane geometry (cup: 35 mm diameter, vane: 28 mm diameter). The system was then cooled to 40° C. hrDecorin (Galacorin™ Catalent, USA) in PBS (4.76 mg/ml) and aqueous sodium chloride (0.2 M) was then added to result in final concentrations of 0.9% (w/v) gellan, 0.24 mg/ml hrDecorin and 10 mM NaCl. Following this, the mixture was cooled at a rate of 1° C./min under shear (450/s) to a final temperature of 20° C. The sample was then removed and stored at 4° C. until further use. In the case of fluid gels without hrDecorin, ratios were adjusted so that the final eye drop had a composition of 0.9% (w/v) gellan, 10 mM NaCl.

### Material Characterization of the Fluid Gel Eye Drops

Microscopy: For transmission microscopy samples were first diluted using polyethylene glycol 400 (PEG400) at a ratio of 1:4 (eye drop to PEG400). Following this, samples were analyzed using an Olympus FV3000. Images were processed using ImageJ (http://imagej.nih.gov/ij/; provided in the public domain by the National Institutes of Health, Bethesda, Md., USA).

For scanning electron microscopy samples were first prepared for lyophilizing by diluting gellan in deionized water in the same manner as for transmission microscopy to a ratio of 1:9. Samples were then rapidly frozen using liquid nitrogen and placed in a freeze drier overnight to leave a powder. Dried sample was then attached to a carbon stub and analyzed using a SEM.

Rheology: Viscosity profiles were obtained using an AR-G2 (TA Instruments, UK) rheometer equipped with sandblasted parallel plates (40 mm, 1 mm gap height) at 20° C. An equilibrium of 2 minutes was used to ensure constant test temperature. Following this, time dependent ramps up and down were applied ranging from 0.1 to 600/s (3 minutes sweep times). Recovery profiles were obtained using the same apparatus, under single frequency. The sample underwent rejuvenation by shearing at 600/s for 10 s. Following this, storage and loss (G′, G″ respectively) were monitored at 1 Hz, 0.5% strain. The cross over point was used as the point at which the sample started to act like a viscoelastic solid.

hrDecorin Release from the Fluid Gel

Levels of hrDecorin release from the gel were determined cumulatively, by placing 1 ml of the fluid gel containing hrDecorin in a 6 well plate. Then 2 ml of DMEM was placed over the sample and the plates were incubated at 37° C. At each time point, the media was removed for measurement of hrDecorin, and replaced with fresh media. Decorin release was quantified using an ELISA specific for human Decorin (R&D systems, Minneapolis, USA) in accordance with the manufacturer's protocol.

In Vitro hrDecorin Bioactivity Assays

Collagen fibrillogenesis: For the dose response curves, 75 □l of PBS was added to each well of a 96 well plate kept on ice. Varying hrDecorin doses were prepared by adding 400 μg/ml of hrDecorin to the first well and subsequently serial diluting (2-fold dilution) across the plate. Following dilution, a further 150 μl of PBS buffer was added to each well. Then, 75 μl of collagen type I (rat tail; Corning, UK) (800 μg/ml) was added to each well and incubated for 2 hours at 37° C. Subsequent absorbance readings were taken using a 405 nm plate reader. Each assay consisted of duplicate blank controls, and triplicate standard dilutions followed by triplicate sample dilutions. Kinetics of fibril formation were determined using a similar setup as the dose response, without serial dilution; incubating the samples within the plate reader, and taking data points every 2 minutes.

### Pseudomonas Keratitis Model and In Vivo Stereomicroscopy

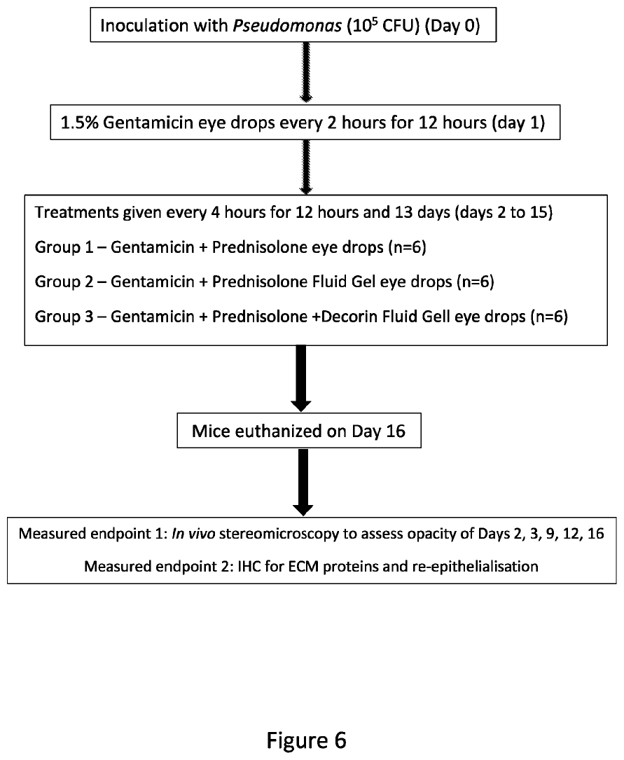

The treatment administration regimes for the in vivo Pseudomonas model are shown in FIG. 6. Groups of naïve intact and infected corneas taken at day 2 were also included in the experimental plan. The sample size of n=6 for each control or treatment group was based on the resource equation^{[49}] as the effect size was unknown. Mice were randomly assigned to each treatment and control group before they were infected with Pseudomonas. Each treatment procedure and the sample sizes are described in further detail below. For the in vivo studies, analyses were performed by investigators masked to the experimental groups.

### In Vivo Murine Model of Pseudomonas Keratitis

P. aeruginosa PAO1 strain was cultured in high salt LB (10 g of tryptone, 5 g of yeast extract, and 11.7 g of NaCl per L, supplemented with 10 mM MgCl₂ and 0.5 mM CaCl₂) at 37° C. for 18 hours. Sub-cultures were derived at an optic density (OD) of 0.2 (OD 650 nm approx. 1×10⁸ CFU/ml). P. aeruginosa were washed (×3) in PBS, centrifuged at 300 rpm for 5 minutes and re-suspended in PBS at a density of 1×10⁵ CFU/2.5 μl. C57BL/6 mice (Jackson Laboratory, CA, USA) were housed in pathogen-free conditions, given free access to food and water and were maintained according to the ARRIVE guidelines, the ARVO statement for the use of animals in ophthalmic and vision research and also adhered to guidelines set out by the University of California, Irvine. For inoculation, mice were anaesthetized and one corneal epithelium was abraded with 3×1 mm parallel scratches using a 26 G needle and inoculated with 2.5 μl P. aeruginosa (1×10⁵ CFU) (strain PAO1)^{64,65}. Mice remained sedated for 2 hours post-inoculation to permit penetration of the infection into the eye, and placed in recovery. After 24 hours, conscious mice were treated with 5 μl of Gentamicin (1.5%, QEHB Pharmacy, Birmingham, UK) every 2 hours for a 12-hour period, to sterilize the infection. After a further 12 hours, mice were administered eye drops (5 μl of each compound) every 4 hours between 8 am and 8 μm for a further 13 days depending on their treatment group: (1) Gentamicin+Prednisolone (0.5%, QEHB Pharmacy), (2) Gentamicin+Prednisolone+Fluid gel, or (3) Gentamicin+Prednisolone+Fluid gel with hrDecorin. Mice were examined for corneal opacification, ulceration and perforation. En-face 24-bit color photographs of the cornea were captured with a SPOT RTKE camera (Diagnostic Instruments) connected to a Leica MZF III stereo Microscope. Mice were euthanized by cervical dislocation under anesthetic at 16 days and eyes enucleated and placed in 4% PFA in PBS for processing for immunohistochemistry.

### Opacity Quantification

Two masked independent ophthalmologists analyzed all photographs in the same randomized order (the order was provided by an independent statistician), for area of opacification using ImageJ. Definitions of corneal opacification, adequate and inadequate images were agreed upon prior to commencement of image analysis by the observers. Measurements were recorded in mm²±SEM. The randomized order dictated that there should be no time-trend in the measured areas.

### Tissue Processing and Immunohistochemistry for Re-Epithelialization and ECM

Enucleated eyes for IHC were post-fixed by immersion in 4% PFA in PBS overnight at 4° C. before cryoprotection using increasing concentrations of sucrose in PBS (10%, 20%, and 30%; Sigma) for 24 hours each at 4° C. Eyes were then embedded in optimal cutting temperature (OCT) embedding medium (Thermo Shandon, Runcorn, UK) in peel-away mold containers (Agar Scientific, Essex, UK) and later sectioned in the parasagittal plane at −22° C. using a cryostat microtome (Bright, Huntingdon, UK) at a thickness of 15 μm, and placed onto Superfrost slides (Fisher Scientific, USA). Central sections (in the optic nerve plane) were used for all IHC studies and stored at −80° C. Frozen sections were left to thaw for 30 minutes before 3×5 minute washings in PBS followed by a 20 minute permeabilization with 0.1% Triton X-100 (Sigma). Non-specific antibody binding sites in tissue sections were blocked for 30 minutes using 0.5% BSA, 0.3% Tween-20 (all from Sigma), and 15% normal goat serum (Vector Laboratories, Peterborough, UK) in PBS before incubating overnight in 4° C. in primary antibody (αSMA, Laminin and fibronectin; 1:200; all from Sigma) again followed by washing 3×5 minutes, and incubating for 1 hour at room temperature with a secondary antibody (Goat anti-mouse Alexa Fluor 488 1:500, Goat anti-mouse Alexa Fluor 594 1:500, Molecular Probes, Paisley, UK). Sections were then washed for 3×5 minutes and mounted in Vectorshield mounting medium containing DAPI (Vector Laboratories). Control tissue sections incubated with secondary antibody alone were all negatively stained.

### Immunohistochemical Imaging and Quantification

After IHC, sections were imaged on a Zeiss Axioscanner fluorescent microscope (Axio Scan.Z1, Carl Zeiss Ltd.) at ×20 using the same exposure times for each antibody. IHC staining was quantified by measuring pixel intensity according to the methods previously described⁶¹. Briefly, the region of interest used for quantitation of ECM IR was defined by a region of interest which was same prescribed size for all eyes/treatments within the stroma. Each stroma had a total of 30 individual intensity measurements (regions of interest) taken to cover the whole area. ECM deposition was quantified within these defined regions of interests and the percentage of IR pixels above a standardized background threshold from intact corneas was calculated using ImageJ. For each antibody, the threshold level of brightness in the area of the stroma was set using intact untreated corneas to define the reference level for test group analysis. Images were assigned randomized file names to ensure masking of treatment groups for the assessor.

Statistical Analysis

All statistical analyses were performed using SPSS 20 (IBM, Chicago, Ill., USA). Normal distribution tests were carried out to determine the most appropriate statistical analysis to compare treatments. Statistical significance was determined at P<0.05. For opacity measurements, corneal width, epithelial thickness, αSMA, Fibronectin and Laminin data were analyzed using ANOVA with Tukey post-hoc tests. For DAPI measurements of epithelial cell layer numbers, as the data were not normally distributed, a Kruskal-Wallis test was used.

TABLE 1
Table 1. Table of treatment groups. Table highlighting the combination of
therapeutics administered (+) or not administered (−) to each group, n = 6.

| | Therapeutic | | | |
|---|---|---|---|---|
| Group No. | Gentamicin | Prednisolone | Fluid gel (FG) | hrDecorin + Fluid gel (DecFG) |
| 1 | + | + | − | − |
| 2 | + | + | + | − |
| 5 | + | + | − | + |

### Further Technical Information

### 1. Biopolymer List:

TABLE 2
Table of biopolymers, both polysaccharide and protein based, with the potential
to be processed into microgel suspensions using a shearing technique. Addition
information of charge, isoelectric point (pl) for proteins, gelling mechanism
and optical clarity has been given - if transparent, the gels has potential
application in ophthalmic devices, however this is not limited to.

| | | | |
|---|---|---|---|
| | | | Gelling |
| Biopolymer | Charge | Optical clarity | mechanism |

Polysaccharides

| | | | |
|---|---|---|---|
| High acyl gellan | −ve | Transparent | Thermal; salt |
| Low acyl gellan | −ve | Transparent | Thermal; salt |
| Agarose | Neutral | Translucent | Thermal |
| Agar | −ve | Slightly translucent | Thermal |
| Chitosan | +ve | Opaque | Thermal |
| Dextran | Neutral | Transparent | Ion cross-linked |
| Xanthan | −ve | Transparent | Thermal; Ion |
| | | | cross-linked |
| Cellulose based | −ve | Transparent | Cross-linked using |
| (CMC HEC) | | | DVS |
| Kappa-carrageenan | −ve | Transparent | Thermal; Ion |
| | | | cross-linked |
| Iota-carrageenan | −ve | Transparent | Thermal; Ion |
| | | | cross-linked |
| High-methoxyl | −ve | Translucent | Ion cross-linked |
| pectin | | | |
| Low-methoxyl pectin | −ve | Translucent | Ion cross-linked |
| Alginate | −ve | Transparent | Ion cross-linked |
| Guar | −ve | Transparent | Thermal; Ion |
| | | | cross-linked |

Proteins

| | | | |
|---|---|---|---|
| Gelatin Type A | pl 7-9 | Transparent | Thermal; salt |
| Gelatin Type B | pl 4.7-5.4 | Transparent | Thermal; salt |
| Whey protein | pl ca. 5 | Dependent on pH | Thermal |
| | | transparent or opaque | |
| Soya protein | pl ca 4.5 | Opaque | Thermal |
| Egg white protein | pl ca 4.5 | Opaque | Thermal |
| Zein | pl ca. 6 | Translucent | Thermal |

1. “Fluid gel” (Micro-Particulate Suspension) Material Properties:

### Viscosity/Flow Behaviours

Optimal eye drop viscosity was sought via two main methods: rheological characterisation of current, commercial eye drops/ointments, and consultation with ophthalmic clinicians. Characterisation of the commercial eye products highlighted a large range of viscosities across both eye drops and eye ointments used to medicate conditions such as dry eye; where optimally long retention times are required. Viscosities were collected and compared at 1 s−1 (chosen as a value within initial stages of shear thinning, so as to avoid artefacts for the apparatus) (Table 2 and FIG. 6 (section A.1.)), highlighting similar viscosities between products as a function of the polymer they were predominately made from: paraffin, carbomer and biopolymer based.

TABLE 1
Table of viscosities derived at 1 s⁻¹ for
commercially available eye drops/ointments.

| Primary Material Type | Upper Viscosity (Pa · s) | Lower Viscosity (Pa · s) |
|---|---|---|
| Paraffin | 200 | 120 |
| Carbomer | 70 | 40 |
| Biopolymer | 10 | 4 |

In the case both paraffin and carbomer based eye products a warning is given within the instructions to inform the patient that blurring and discomfort could arise due to the drops. As such, the outer limits of the formulation viscosities have been founded based on the values obtained for these products:

Maximum—200 Pa·s; and Minimum 4 Pa·s

In all cases, with all formulations tested, these values have not been exceeded. Thus, all formulations prepared with the gellan as the biopolymer used for gelation could be used within these limits. However, a more optimal formulation, in terms of viscosity, was narrowed upon with aid of clinical advice.

Having made a panel of different formulations, clinicians were asked to manipulate the product and rate them in respect to plausible eye drop products. From this data, it was found that the eye drops in the viscosity range:

5 to 50 Pa·s

were more easily applied, with good retention, with the optimal drop:

ca. 10 to 20 Pa·s.

Furthermore, the system should exhibit shear thinning behaviour.

### 1.1.1. Defined Parameters

TABLE 2
Summary of potential viscosities as found at 1
s⁻¹ (20° C.) for eye drop formulations.

| | Maximum Range | | Narrowed Range | | Optimal | |
|---|---|---|---|---|---|---|
| Parameter | Lower | Upper | Lower | Upper | Lower | Upper |
| Viscosity | 4 | 200 | 5 | 50 | 10 | 20 |
| (Pa · s) | | | | | | |

Shear thinning

### Elasticity

Elasticity at rest plays a large role in the use of the product to be retained and deliver actives in a controlled way. It is believed that the ability of the microgel suspension to create a weak elastic network at rest drives the high retention times related to the product. Again, the limits are based upon the characterisation of commercially available eye drops and ointments (FIG. 3 (section A.1.)). A similar correlation was observed between the various products as was seen for the viscosities, with the products being grouped into polymer type (Table 4).

TABLE 3
Table of storage (elastic moduli) derived using amplitude
sweeps for commercially available eye drops/ointments.

| Primary Material Type | Upper G′ (Pa) | Lower G′ (Pa) |
|---|---|---|
| Paraffin | ca. 20000 | ca. 20000 |
| Carbomer | 300 | 200 |
| Biopolymer | 20 | 14 |

Again, similar to the viscosity, for all formulations tested, the values obtained for current products were not exceeded. Thus, all formulations prepared with the gellan as the biopolymer used for gelation could be used within these limits.

Maximum—20000 Pa; and Minimum 1 Pa

However, when analysed by the clinicians this was narrowed to

### 1 to 250 Pa,

with the optimal formulation ranging between

### 20 to 40 Pa.

### 1.1.2. Defined Parameters

TABLE 4
Summary of potential elastic moduli as found within
the linear viscoelastic region (LVR) at 1 Hz (20°
C.) using a strain sweep for eye drop formulations.

| | Maximum Range | | Narrowed Range | | Optimal | |
|---|---|---|---|---|---|---|
| Parameter | Lower | Upper | Lower | Upper | Lower | Upper |
| Storage/Elastic | 1 | 20000 | 1 | 250 | 20 | 40 |
| Modulus (Pa) | | | | | | |

pH

Due to the chemical composition of biopolymers, and varying chemical moieties along their individual backbones, they have varying natural pH's. The pH of products coming into contact with the ocular surface is important, as many chemical injuries are formed in the range of pH<4 and pH>10, with normal physiology close to 7.11±1.5. Therefore, eye drops are formulated in between this range (4-10), with some products dropping as low as pH 3.5 (proparacaine hydrochloride solution)′. Therefore, based on this data from literature the eye drop formulations should have a pH within the range of:

3.5 to 8.6

However, delivery of many actives including proteins requires the formulation to be neutral. In these cases, PBS (phosphate buffered saline) can be added to the eye drop, restricting the pH to a neutral acidity. Therefore, in the formulation has been narrowed to a pH:

6.5 to 7.5

with the optimised formulation being:

7.4.

### 1.1.3. Defined Parameters

TABLE 5
Summary of potential pH's for eye drop formulations.

| | Maximum Range | | Narrowed Range | | Optimal | |
|---|---|---|---|---|---|---|
| Parameter | Lower | Upper | Lower | Upper | Lower | Upper |
| pH | 3.5 | 8.6 | 6.5 | 7.5 | 7.4 | 7.4 |

### 2. “Fluid Gel” (Micro-Particulate Suspension) Formulation:

### Biopolymer Concentration

### (See Experimental Write-Up A.1.)

Ultimately the material properties of the formulations are governed by the concentration of initial polymer in the product. Therefore, the upper and lower material properties were used to evaluate the material formulations, providing upper and lower limits for polymer concentrations. As all systems exhibited shear thinning behaviours limits were solely based on fulfilling the criteria for both viscosity (at 1 s⁻¹) and elastic behaviour at rest. Thus, the maximum range of:

0.5 to 2.5% (w/v)
has been set for eye drop formulations, with values within those found for commercially available products. This have been narrowed to fall within the clinician's advice to:
0.5 to 1.5% (w/v)
with the optimised formulation consisting of:
0.9% (w/v)

### 2.1.1. Defined Parameters

TABLE 6
Summary of potential pH's for eye drop formulations.

| | Maximum Range | | Narrowed Range | | Optimal | |
|---|---|---|---|---|---|---|
| Parameter | Lower | Upper | Lower | Upper | Lower | Upper |
| Gellan Conc. | 0.5 | 2.5 | 0.5 | 1.5 | 0.9 | 0.9 |
| (% (w/v)) | | | | | | |

### Cross-Linker Concentration

### (See Experimental Write-Up A.2.)

Data obtained from characterisation of the gellan formulations showed that salt content did not influence the viscosities of the systems, however, it did have an effect on the elastic response of the gel at rest. Again, none of the systems formulated exceeded the upper and lower limits set by the commercial products as such upper and lower concentrations have been defines as:

5 to 40 mM

However, the mechanical spectra showed that at higher salt concentrations, a marked reduction in the elastic network was formed when deformed outside the linear viscoelastic region. This suggests that lower salt concentrations result in more plastic behaviour, which is believed would be more comfortable for the patient. Therefore, the narrowed limits for the formulation have been adjusted to;

5 to 20 mM

with the optimised formulation being: 10 mM.

### 2.1.2. PBS

The addition of PBS can be used to manipulate the pH of the system. In these cases, 5% v/v (5% was determined as the amount added with the therapeutic decorin, so further studies have not been conducted in this area) is added which will affect the level of salt within the system. The concentration of mono-valent ions within the PBS has been calculated and tabulated in Table 8.

TABLE 7
Table highlighting constituents and concentrations of PBS.

| Chemical | Concentration (M) | Concentration at 5% |
|---|---|---|
| Phosphate Buffer | 0.01 | — |
| KCl | 0.0027 | 0.14 mM |
| NaCl | 0.137 | 6.8 mM |
| | Total extra ion content | Ca. 7 mM |

Therefore, the range for the cross-linker has been altered (Table 9), reducing the lower limit as the ion content in the PBS is sufficient to drive the gelation process.

2.1.3. Defined Parameters

TABLE 8
Summary of cross-linker concentrations
for eye drops with and without PBS.

| | Maximum Range | | Narrowed Range | | Optimal | |
|---|---|---|---|---|---|---|
| Parameter | Lower | Upper | Lower | Upper | Lower | Upper |
| NaCl conc. | 5 | 40 | 5 | 20 | 10 | 10 |
| without PBS | | | | | | |
| (mM) | | | | | | |
| NaCl conc. | 0 | 40 | 0 | 20 | 10 | 10 |
| with PBS | | | | | | |
| (mM) | | | | | | |

3. “Fluid gel” (Micro-Particulate Suspension) Processing Parameters:

Thermal Processes

The thermal processing within the manufacture are key to formation of a gel. Typically, the thermal parameters are divided into two sections: the processing temperatures, and the rate of cooling.

4.1.1. Processing Temperatures

The inlet and outlet, are key to make sure the polymer is in a sol prior to processing and exits at a temperature below the gelling transition. Initially the inlet temperature was set as close to the gelling temperature as possible, due to the protein active denaturing at higher temperatures. Therefore, this was set to 40° C. However, this is not necessary, the key aspect to the inlet temperature is to keep it above the gelling temperature, to prevent early gelation and blockages. The function of the outlet temperature is to ensure ordering/structuring of the polymer has completed prior to storage. This prevents aggregation during stage and heterogeneous suspensions forming. As such, for gellan this temperature has been defined as 20° C., allowing the polymer to pass though the gelation process. The exit temperature is therefore controlled by the jacket of the mill, which is set to provide sufficient cooling during the process. This can be changed resulting in various cooling rates.

### 4.1.2. Cooling Rate

### (See Experimental Write-Up A.3.)

The cooling rate during the sol-gel transition is known to be very important in regard to the final material properties; as higher cooling rates result in rapid formation of structures and weaker overall moduli. This was observed for the microgel suspensions, however, only at higher polymer concentrations. It was observed that for the optimal eye drop formulation, no changes in material properties were observed suggesting that a large range of parameters could be used:

0.1 to 6° C.min⁻¹

Whereas at 1.8% w/v polymer it was more dependent on elastic structure required.

### 4.1.3. Defined Parameters

TABLE 9
Summary of cooling rates for the gellan eye drop formulation.

| | Maximum Range | | Narrowed Range | | Optimal | |
|---|---|---|---|---|---|---|
| Parameter | Lower | Upper | Lower | Upper | Lower | Upper |
| Cooling rate | 0.1 | 6 | 0.5 | 1 | 0.5 | 0.7 |
| (° C. min⁻¹) | | | | | | |

### 4.2 Shearing Rate

### (See Experimental Write-Up A.3.)

Shear rate during processing showed very similar results to cooling rate, with the optimised polymer concentration un-effected by the processing shear. Again, the higher concentrations showed a dependency. Therefore, for the optimised formulation a very broad range of shear could be applied:

50-2000 rpm (limits of the equipment)

which can be narrowed to:

500 to 1500 rpm

with the optimised setting:

1000 rpm (to prevent stressing on the processing equipment)

### 4.2.1 Defined Parameters

TABLE 10
Summary of processing rates for the gellan eye drop formulation.

| | Maximum Range | | Narrowed Range | | Optimal | |
|---|---|---|---|---|---|---|
| Parameter | Lower | Upper | Lower | Upper | Lower | Upper |
| processing rate | 50 | 2000 | 500 | 1500 | 1000 | 1000 |
| (rpm) | | | | | | |

### 5. Summary of Suspension Parameters:

TABLE 11
Summary of potential pH's for eye drop formulations.

| | Maximum Range | | Narrowed Range | | Optimal | |
|---|---|---|---|---|---|---|
| Parameter | Lower | Upper | Lower | Upper | Lower | Upper |
| Viscosity | 4 | 200 | 5 | 50 | 10 | 20 |
| (Pa · s) | | | | | | |
| | Shear thinning | | | | | |
| Storage/Elastic | 1 | 20000 | 1 | 250 | 20 | 40 |
| Modulus (Pa) | | | | | | |
| pH | 3.5 | 8.6 | 6.5 | 7.5 | 7.4 | 7.4 |
| Gellan Conc. | 0.5 | 2.5 | 0.5 | 1.5 | 0.9 | 0.9 |
| (% (w/v)) | | | | | | |
| NaCl conc. | 5 | 40 | 5 | 20 | 10 | 10 |
| without PBS | | | | | | |
| (mM) | | | | | | |
| NaCl conc. | 0 | 40 | 0 | 20 | 10 | 10 |
| with PBS | | | | | | |
| (mM) | | | | | | |
| Cooling rate | 0.1 | 6 | 0.5 | 1 | 0.5 | 0.7 |
| (° C. min⁻¹) | | | | | | |
| processing rate | 50 | 2000 | 500 | 1500 | 1000 | 1000 |
| (rpm) | | | | | | |

### Further Experimental Data

### A.1. Experiment—Gellan Concentration: The Effects of Polymer Concentration on Resulting Fluid Gel Material Responses

### Aims:

Understand how polymer concentration effects the overriding material properties (viscosity and elasticity) following processing into a microgel suspension.
Narrow polymer concentration tolerances for suitable eye drop formulations.

### Materials and Methods:

### Materials:

Gellan (Kelco,)
NaCl (Fisher Chemicals, Lot No.: 1665066)

### Preparation of Gellan Microgel Suspensions (MS):

### Preparation of Stock Solutions:

### Preparation of NaCl Solution:

NaCl (0.2 M) was prepared through the addition of dry crystals (1.16 g) to deionised water (100 ml) using a volumetric flask. The NaCl was then allowed to dissolve using an inverting technique to aid the process. Once fully dissolved the solution was kept at ambient conditions until further use.

### Preparation of Gellan Sols:

Gellan sols were prepared by dissolving powdered polymer into water/NaCl solution at varying ratios so that the final concentrations post-processing were equal to 0.5, 0.9, 1.35, 1.8 and 2.35% (w/v). In brief, gellan powder was weighed out (2.5, 4.5, 6.75, 9.0 and 11.75 g) and added to 450 ml of deionised water. The mixture was allowed to heat to 95° C. under agitation, allowing the polymer to dissolve. Once fully dissolved, 25 ml of NaCl stock solution (0.2 M) was added to the solution resulting in a 10 mM concentration post-processing. The sol was then allowed to reach thermal equilibrium at 95° C. before processing.

### Processing of Gellan MS:

MS were prepared using a jacketed pin mill set to 20° C. Gellan sols were pumped using a peristaltic pump into the pin mill at 3 ml/min so that it entered the processing chamber at 40° C. Prior to entry using a syringe and syringe pump, water was pumped into the gellan stream (at a rate of 0.16 ml/min) so that they impinged, diluting the gellan sol to the final concentrations (0.5, 0.9, 1.35, 1.8 and 2.35% (w/v), 10 mM NaCl). The mixture was then cooled under shear (500 rpm or 1000 rpm) as it passed through the milling unit. On exiting, at 20° C., the gel was packaged and stored at 4° C. until further testing.

### Material Analysis:

### Rheometry:

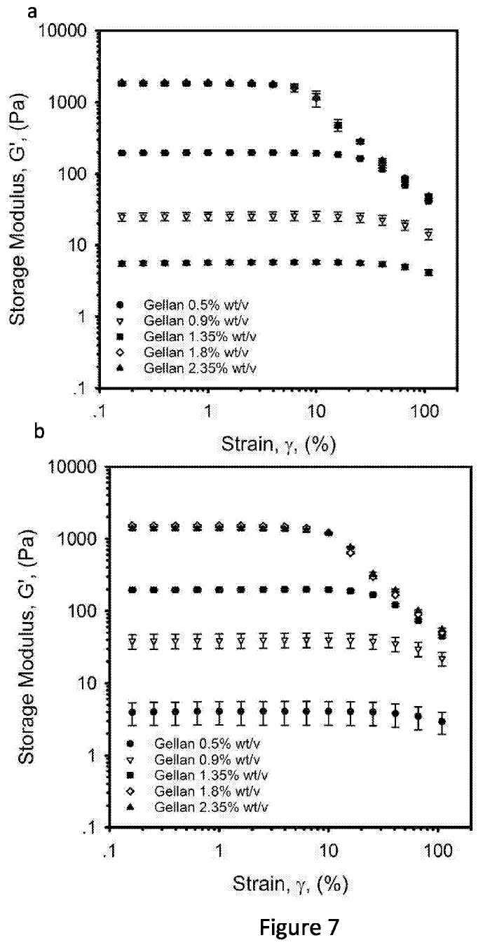

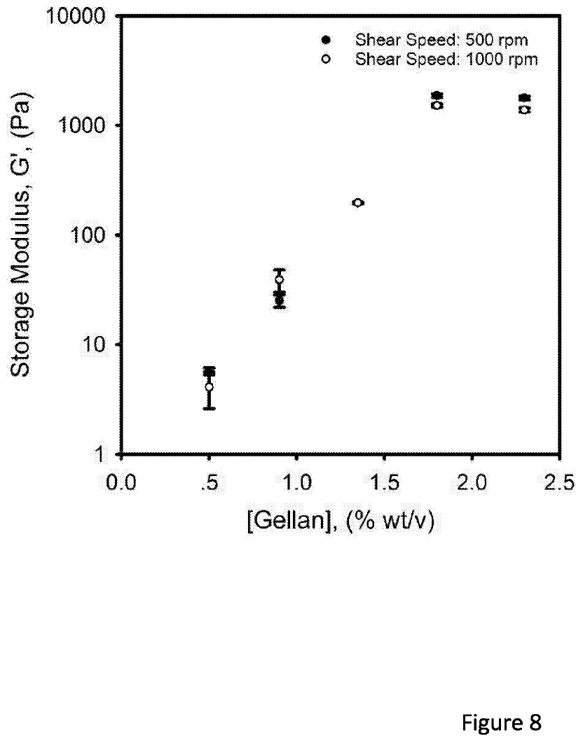

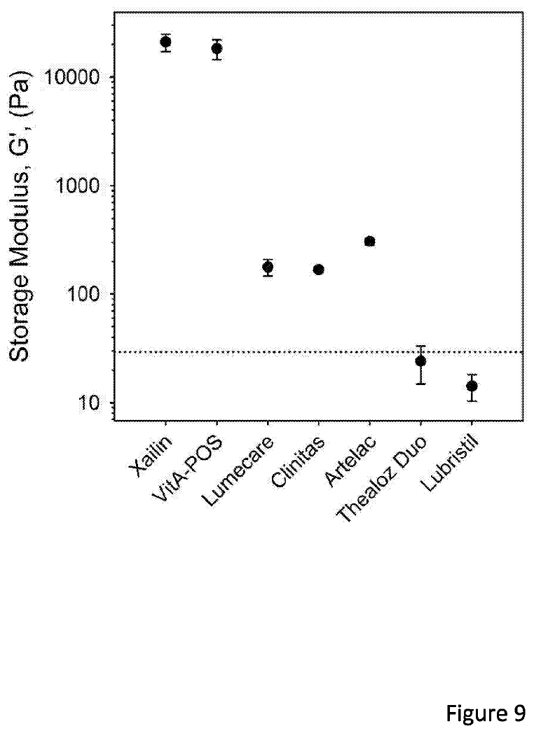

A rheometer (TA, AR-G2) equipped with a sandblasted parallel plate (40 mm diameter, 1 mm gap height) was used to test all samples, at 20° C. Results are shown in FIGS. 7 to 9

### Amplitude Sweeps:

Amplitude sweeps were obtained in strain controlled mode over a range of 0.1 to 100.0%. Samples were loaded into the instrument and upper geometry lowered. Once trimmed, the sample was left to equilibrate at 20° C. prior to testing. Measurements were obtained at 1 Hz in a logarithmic fashion.

### Flow Profiles:

Viscosity profiles for the samples were obtained using a continuous ramp. Samples were loaded into the instrument and upper geometry lowered. Once trimmed, the sample was left to equilibrate at 20° C. prior to testing. Increasing shear was applied to the sample in rate controlled mode, between 0.1 and 600 s⁻¹ over a 3-minute ramp, with data points obtained in a logarithmic fashion.

### Results:

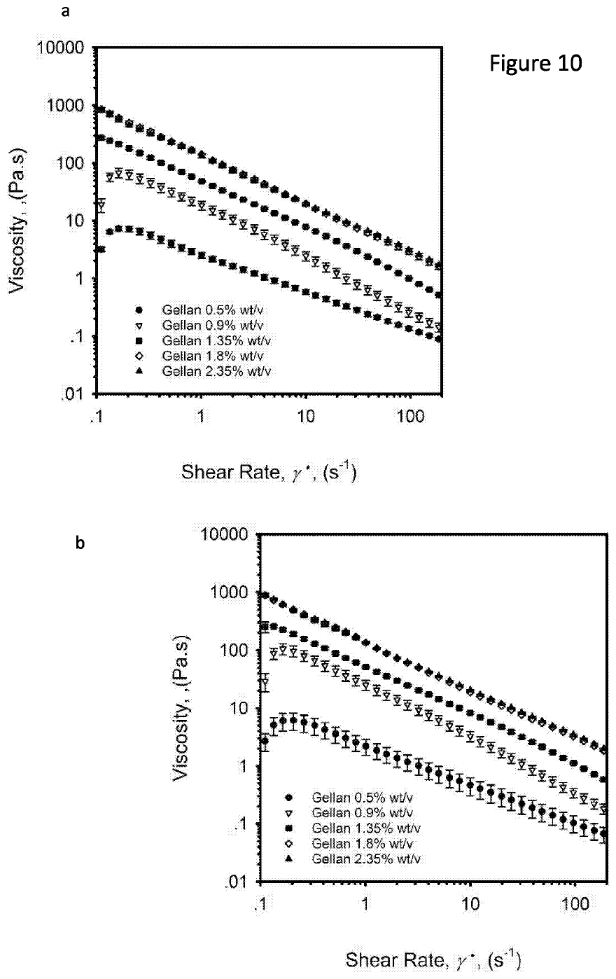

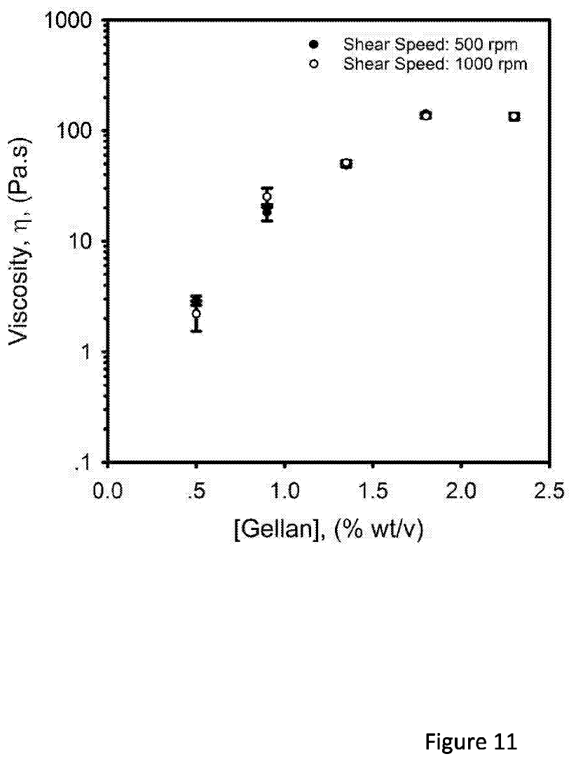

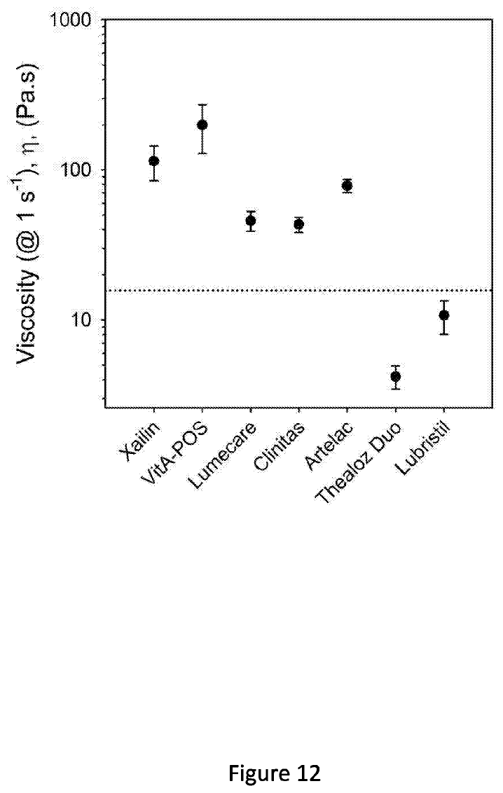

Small deformation rheology: see FIGS. 7 to 9 and discussion above.
Large deformation rheology: see FIGS. 10 to 12 and discussion above.

### Discussion:

The effects of polymer concentration can be observed for both their elastic nature and viscosity, with both properties, showing the same trend; increasing until a plateau is reached at concentrations above 1.8% (w/v) (FIGS. 2 and 3). Such observations arise from the formation of micro-gelled particles, where by applying shear throughout the gelation of the polymer systems, confinement prevents a continuous network form forming. The overriding result of this process means that the gelled entities are dispersed in a non-gelled medium, similarly to a W₁/W₂ emulsion. As such, the rheology of the suspensions also closely correlates to those of emulsions; where increasing the phase volume of the droplets or particles (in this case) results in closer proximity and an increase in both the elastic nature (G′) and viscosity of the systems. In this case, increasing the polymer concertation results in a larger number of particles, until a maximum packing fraction is reached. Above this, no further changes in material properties are seen.

Elasticity (storage modulus, G′) and viscosity of the various suspensions were compared to data collected for current eye drops/ointments, across a range of materials: Paraffin, carbomer and biopolymer based systems (FIGS. 3 and 6). It was observed that all the gellan systems exhibited G′ and viscosities within the thresholds of the current commercial eye products, suggesting that all systems would be suitable irrespective of polymer concertation. However, for ease of application (from single use applicators), and comfort (blurred vision as described by the packaging) values closest to the carbomer and biopolymer based drops were optimum. Therefore, gellan concentrations in the range of 0.5 to 1.35% (w/v) were most suitable. Additionally, consultation with independent clinicians with a view for ocular application resulted in the 0.9% (w/v), most closely mimicking the properties defined by the clinicians.

Yielding behaviours of the suspensions are also very important, especially within retention mechanisms for delivery, as rapidly yielding systems result in fast clearance. Vice versa, where systems do not yield at all, materials are not readily eliminated from the body. The linear viscoelastic region (LVR) is a good indication of the yielding behaviours of the suspensions, as systems leave this linear region, the weak inter-particle interactions begin to break down and the systems flows. The length of the LVR was observed to be a function of the polymer concentration, showing an inverse relationship to gellan content (FIG. 1). Here, at lower polymer concentrations the suspension can be manipulated at higher strains before breaking down, providing an occlusive barrier in dynamic regions of the body that becomes slowly resorbed. Similar LVRs are observed in the range of 0.5 to 1.35% (w/v) suggesting that they would behave similarly.

Following yielding shear thinning behaviour is also vital for both application and elimination, allowing the suspensions to easily flow upon liquefaction. Shear thinning was observed across all systems, irrespective of polymer concentration (FIG. 4). A high degree of shear thinning, arising through the breakdown of inter-particle interactions and alignment in flow allows the systems to be easily applied through a nozzle (syringe, single use applicator etc.); where small pressures result in high levels of shear.

### Conclusions:

In summary, it was shown that polymer concentration played a key role in the resulting material properties of the gellan microgel suspensions. Material characteristics such as elasticity, represented by the materials intrinsic G′ values, and viscosity were found to be a function of the polymer concentration, increasing until a plateau was formed at 1.8% (w/v). Effectively this meant that all systems were suitable for application within the ocular environment or injection, comparing closely with already commercially available products and demonstrating strong shear thinning behaviours needed for extrusion through small orifices. Furthermore, by comparing to commercial products and through conversations with independent clinicians, a polymer range between 0.5 and 1.35% (w/v) was narrowed down with 0.9% (w/v) proving optimal for the final formulation.

### A.2. Experiment—Cross-Linker (NaCl) Concentration: The Effects of Cross-Linker Concentration on Resulting Fluid Gel Material Responses.

### Aims:

Understand how the cross-linker concentration effects the overriding material properties (viscosity and elasticity) following processing into a microgel suspension.
Narrow cross-linker concentration tolerances for suitable eye drop formulations.

### Materials and Methods:

Gellan (Kelco,)
NaCl (Fisher Chemicals, Lot No.: 1665066)

### Preparation of Gellan Microgel Suspensions (MS):

### Preparation of Stock Solutions:

### Preparation of NaCl Solutions:

NaCl (0.1, 0.2, 0.4 and 0.8 M) were prepared through the addition of dry crystals (0.58, 1.16, 2.32 and 4.64 g) to deionised water (100 ml) using a volumetric flask. The NaCl was then allowed to dissolve using an inverting technique to aid the process. Once fully dissolved the solutions were kept at ambient conditions until further use.

### Preparation of Gellan Sols:

Gellan solutions were prepared by dissolving powdered polymer into water/NaCl solution so that the final concentrations post-processing were equal to 0.9% and 1.8% (w/v). In brief, gellan powder was weighed out (4.5 g, 9.0 g) and added to 450 ml of deionised water. The mixture was allowed to heat to 95° C. under agitation, allowing the polymer to dissolve. Once fully dissolved, 25 ml of NaCl stock solution (either 0.1, 0.2, 0.4 and 0.8 M) was added to the solution resulting in a 5, 10, 20 or 40 mM concentration post-processing. The sol was then allowed to reach thermal equilibrium at 95° C. before processing.

### Processing of Gellan MS:

MS were prepared using a jacketed pin mill set to 20° C. Gellan sols were pumped using a peristaltic pump into the pin mill at 3 ml/min so that it entered the processing chamber at 40° C. Prior to entry, using a syringe and syringe pump, water was pumped into the gellan stream (at a rate of 0.16 ml/min) so that they impinged, diluting the gellan sol to the final concentrations (0.9% and 1.8% (w/v); 5, 10, 20 or 40 mM NaCl). The mixture was then cooled under shear (1000 rpm) as it passed through the milling unit. On exiting, at 20° C., the gel was packaged and stored at 4° C. until further testing.

### Material Analysis:

### Rheometry:

A rheometer (TA, AR-G2) equipped with a sandblasted parallel plate (40 mm diameter, 1 mm gap height) was used to test all samples, at 20° C.

### Amplitude Sweeps:

Amplitude sweeps were obtained in strain controlled mode over a range of 0.1 to 100.0%. Samples were loaded into the instrument and upper geometry lowered. Once trimmed, the sample was left to equilibrate at 20° C. prior to testing. Measurements were obtained at 1 Hz in a logarithmic fashion.

### Flow Profiles:

Viscosity profiles for the samples were obtained using a continuous ramp. Samples were loaded into the instrument and upper geometry lowered. Once trimmed, the sample was left to equilibrate at 20° C. prior to testing. Increasing shear was applied to the sample in rate controlled mode, between 0.1 and 600 s⁻¹ over a 3-minute ramp, with data points obtained in a logarithmic fashion.

### Results:

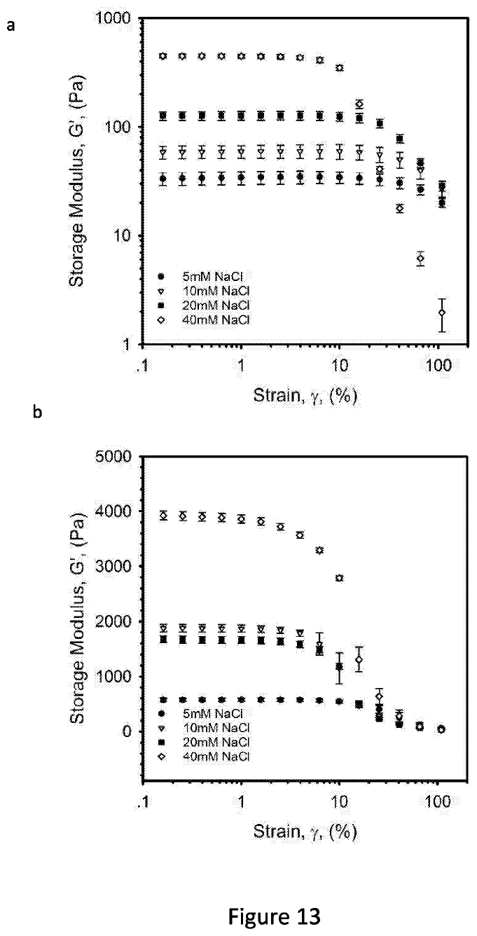

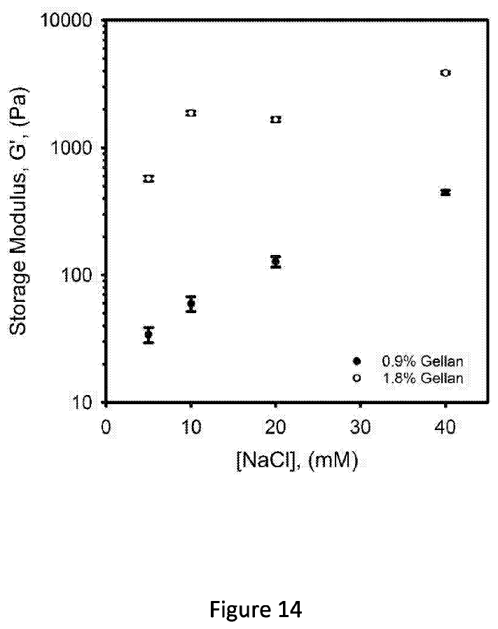

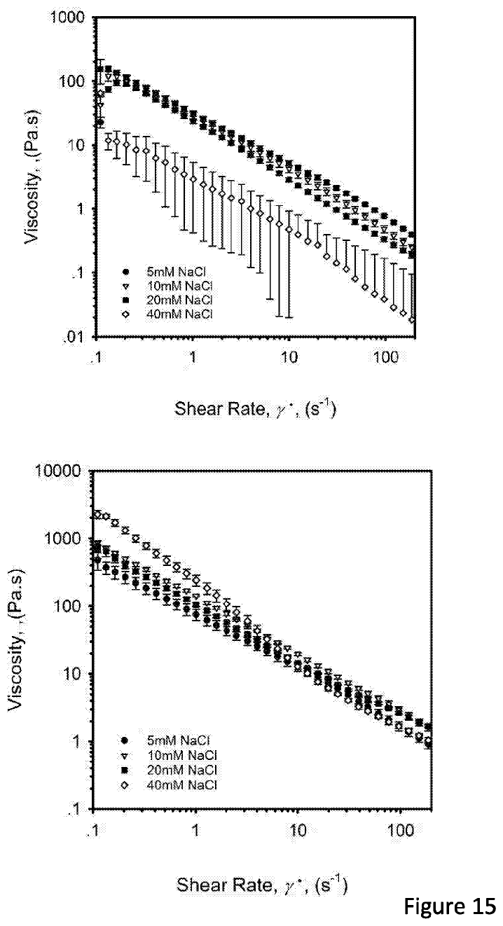

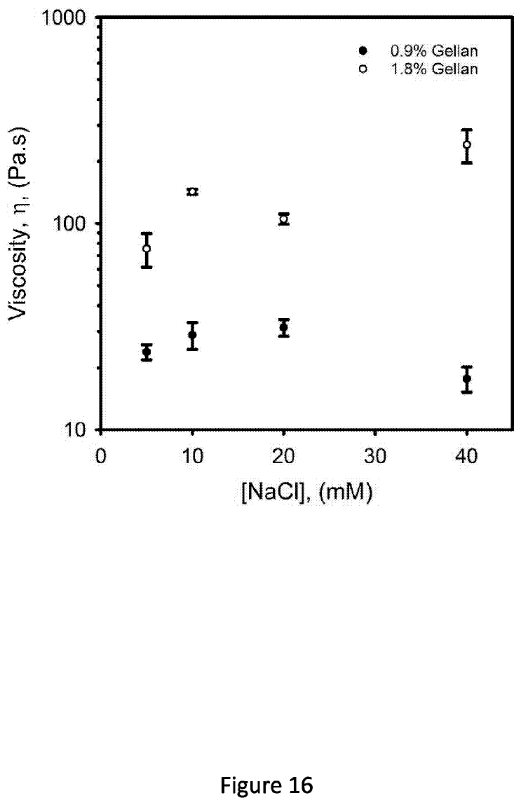

Small deformation rheology—see FIGS. 13 to 14
Large deformation rheology—see FIGS. 15 to 16

### Discussion:

Mechanistically, salts play a vital role in the gelation of many polymers, including gellan. Salt type, particularly valency (mono, di, tri etc.) are key to the resultant gel properties; typically, increasing the valency increases the gel strength, as more bridges are formed between polymers. However, in the case of gellan, di-valent ions, e.g. Ca²⁺, results in clouding (increased turbidity) of the resultant gel. As such mono-valent ions such as Na⁺ can be used to strengthen the junction sites between helices, forming the 3-dimensional gel structure. Therefore, resultant gel strength is a function of concentration of salt, also termed cross-linker, added. The effects of cross-linker concentration on formation and resultant microgel suspensions (“fluid gels”) formed through sheared gelation can be clearly seen in FIGS. 1 and 2. Here, a correlation between NaCl concentration and elastic (G′) response can be observed, corresponding to known gelation mechanisms (increased strength for higher cross-linker concentrations), for both polymer concentrations studied (FIG. 2). Furthermore, the mechanical spectra (FIG. 1) highlights a change in material yielding properties. It was observed that at the highest salt concentration (40 mM) the materials strain dependency increased, showing a more rapid decrease in G′ on leaving the LVR (linear viscoelastic region). Such observations fit closely with typical material responses, as gel becomes stronger it becomes more brittle. In these cases, it is believed that as the system become more densely crosslinked the material behaves closer to fracturing as it reaches a critical strain, as opposed to plastically deforming. Although, the higher concentrations are spreadable, the enhanced strain dependency detracts from their use in applications such as ocular, where increased plasticity on deformation results in a smoother surface, with expected improvements in acuity and comfort.

The effects of salt concentration on the viscosity of the eye drop was also tested. Little changes were observed across all systems (FIG. 2), with all formulations showing marked shear thinning behaviours with overall viscosity ultimately dependent on the biopolymer concentration. However, it was observed at 0.9% (w/v) gellan with the highest salt concentration (40 mM), that the overall viscosity of the suspension was lower. Such an observation was accompanied with increased error, potentially arising from a degree of syneresis (expulsion of water), where the increase cross-link density pulled the polymers closer, resulting in insufficient polymer to structure the water phase. Therefore, the stability of these systems is potentially compromised, leading to heterogeneous systems over time.

### Conclusions:

In summary, the addition of salt to the biopolymer system resulted in manipulation over the strength of the final product. Increasing salt concentrations ultimately increased the number of crosslinks in the system and final material elastic behaviour. Additionally, such effects were not seen to have drastic changes in viscosity, however, at lower polymer concentrations too much cross-linking could lead to the formation of heterogeneous suspensions and poor stability. Probing the elastic structure using strain sweeps allowed the yielding behaviour of the suspensions to be analysed, highlighting higher strain dependencies at 40 mM formulations. The reduction in plastic nature is expected to arise in discomfort for the patient in ocular applications, thus, the upper limit for cross-linker is suggested to be 20 mM.

### A.3. Experiment—Cooling Rate: The Effects of Cooling Rate Applied During Processing in Regard to the Manufacture of Gellan Microgel Suspensions (“Fluid Gels”).

### Aims:

Understand the role that cooling rate plays on the resultant material properties (viscosity and elasticity) of the gellan microgel suspensions.
Narrow the cooling rate to tolerances suitable eye drop processing.

### Materials and Methods:

Gellan (Kelco,)
NaCl (Fisher Chemicals, Lot No.: 1665066)

### Preparation of Gellan Microgel Suspensions (MS):

### Preparation of Stock Solutions:

### Preparation of NaCl Solutions:

NaCl (0.2 M) was prepared through the addition of dry crystals (1.16 g) to deionised water (100 ml) using a volumetric flask. The NaCl was then allowed to dissolve using an inverting technique to aid the process. Once fully dissolved the solutions were kept at ambient conditions until further use.

### Preparation of Gellan Sols:

Gellan solutions were prepared by dissolving powdered polymer into water/NaCl solution so that the final concentrations post-processing were equal to 0.9% and 1.8% (w/v). In brief, gellan powder was weighed out (4.5 g, 9.0 g) and added to 475 ml of deionised water. The mixture was allowed to heat to 95° C. under agitation, allowing the polymer to dissolve. Once fully dissolved, 25 ml of NaCl stock solution (0.2 M) was added to the gellan sol, resulting in a 10 mM final concentration. The sol was then allowed to reach thermal equilibrium at 95° C. before processing.

### Processing of Gellan MS:

MS were prepared using a jacketed pin mill, whereby the jacket temperature and the residence time within the mill were altered to result in cooling rates of 1, 3 and 6° C.min⁻¹. As an example: the jacket was set to 5° C. and with a flow rate of 20 mlmin⁻¹, the temperature of the fluid at the inlet was 46 and outlet 16, the residence time at this rate was 5 minutes, thus the cooling rate was equal to 6° C.min⁻¹. On exiting, the gel was packaged and stored at 4° C. until further testing.

### Material Analysis:

### Rheometry:

A rheometer (TA, AR-G2) equipped with a sandblasted parallel plate (40 mm diameter, 1 mm gap height) was used to test all samples, at 20° C.

### Amplitude Sweeps:

Amplitude sweeps were obtained in strain controlled mode over a range of 0.1 to 100.0%. Samples were loaded into the instrument and upper geometry lowered. Once trimmed, the sample was left to equilibrate at 20° C. prior to testing. Measurements were obtained at 1 Hz in a logarithmic fashion.

### Flow Profiles:

Viscosity profiles for the samples were obtained using a continuous ramp. Samples were loaded into the instrument and upper geometry lowered. Once trimmed, the sample was left to equilibrate at 20° C. prior to testing. Increasing shear was applied to the sample in rate controlled mode, between 0.1 and 600 s⁻¹ over a 3-minute ramp, with data points obtained in a logarithmic fashion.

### Results:

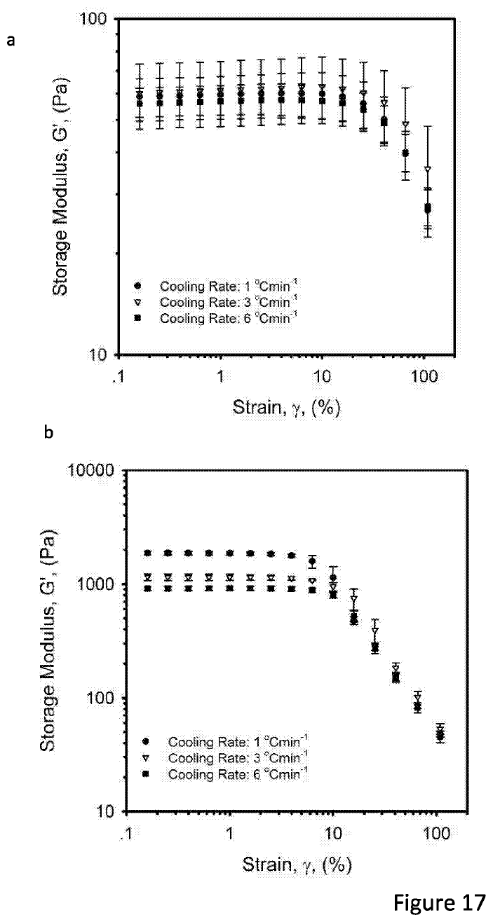

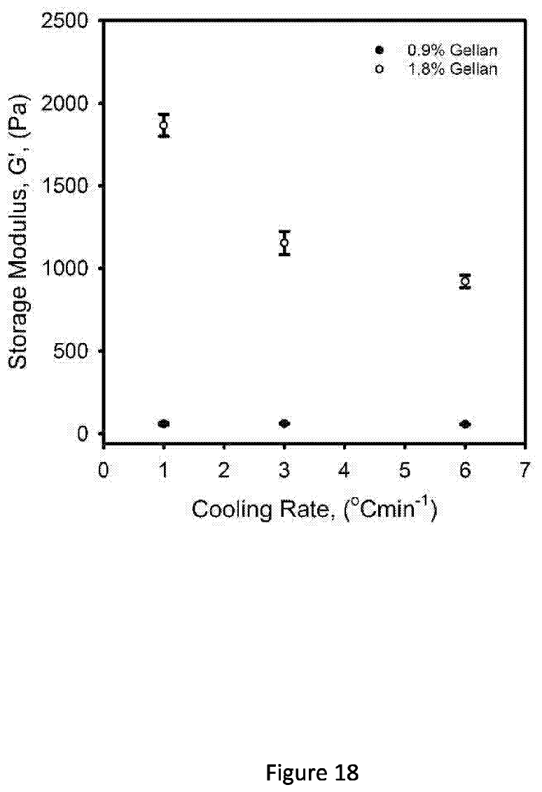

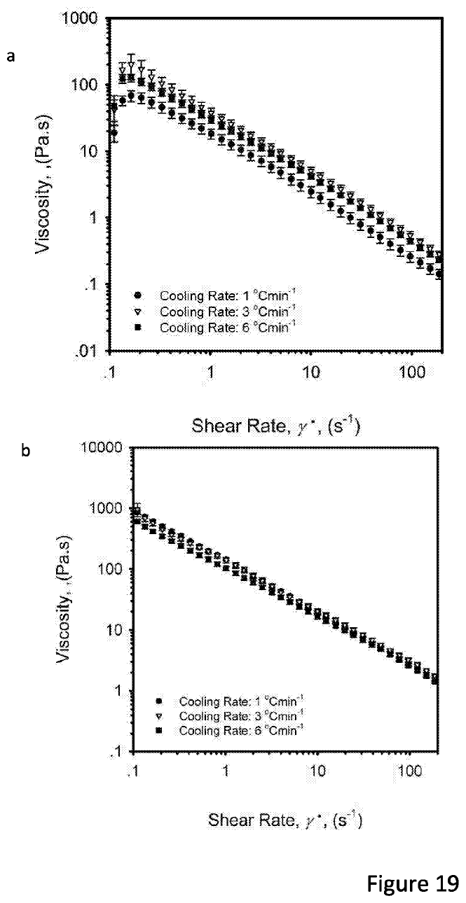

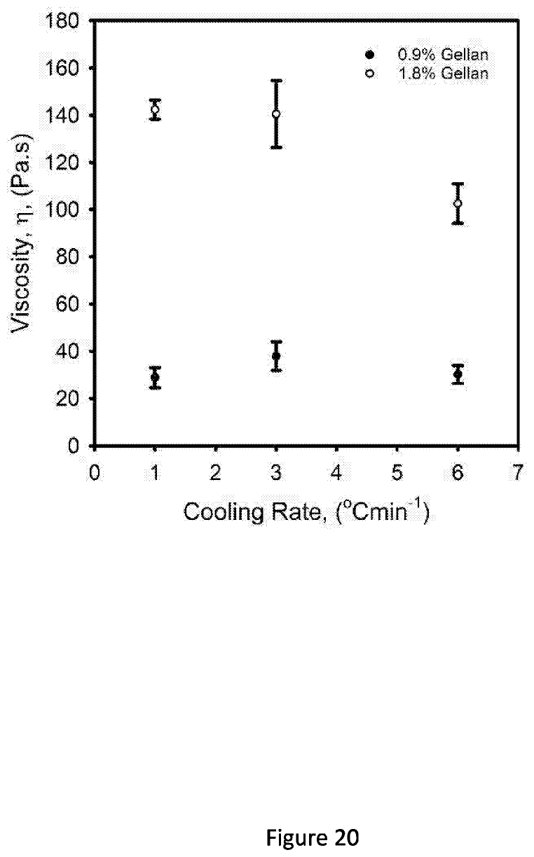

Small deformation rheology: See FIGS. 17 and 18
Large deformation rheology: See FIGS. 19 and 20

### Discussion:

Cooling plays a key role in the formation of gellan hydrogels, forcing the polymers through a random coil to helix transition. The effects of cooling rate on the formation of fluid gels was studied to evaluate related changes in material response. It was observed that at the lower polymer concentration (0.9% (w/v)) the cooling rate had little effect on both the degree of elasticity within the system and overall viscosity. However, at higher concentrations (1.8% (w/v)), the cooling rate has a much more pronounced effect on the elastic modulus (G′) (FIG. 2). It is believed, that at higher polymer concentrations particles are held in much closer proximity, as such are effected much more by particle deformation. The slower cooling rate allows the particles to form much more slowly, resulting in more ordered, stronger structures. Little effect is observed for the viscosity however, suggesting that particles interact with each other to a similar extent, with particles characterised on the microscale as they “squeeze” past each other.

The data obtained suggests an extra degree of control over the material properties at higher polymer concentrations. Being able to engineer specific elastic properties to the system without changing the overall viscosity. This is important within delivery systems to various areas of the body, allowing a semi-solid like structure to be placed in situ that provides a barrier or prolonged retention. Furthermore, the ability to retain the same viscosity means that even though it acts more solid like at rest, the system remains injectable.

### Conclusions:

The effects of cooling rate were found to be dependent on the polymer concentration, with the optimised eye drop formulation found to be independent on the cooling rate applied. However, at higher concentrations, the elastic structure can be finely tuned, without effecting the viscosity profiles. As such the degree of solidity can be manipulated when the system is at rest, but remains flowable (syringable) at larger deformation.

### A.4. Experiment—Mixing Speed Applied on Processing: The Effects of Mixing Speed Applied During Processing in Regard to the Formulation of Gellan Microgel Suspensions (“Fluid Gels”).

### Aims:

Understand the role that mixing speed during processing plays on the resultant material properties (viscosity and elasticity) of the gellan microgel suspensions.
Narrow the speed of mixing during processing to tolerances suitable eye drop formulations.

### Materials and Methods:

Gellan (Kelco,)
NaCl (Fisher Chemicals, Lot No.: 1665066)

### Preparation of Gellan Microgel Suspensions (MS):

### Preparation of Stock Solutions:

### Preparation of NaCl Solutions:

NaCl (0.2 M) was prepared through the addition of dry crystals (1.16 g) to deionised water (100 ml) using a volumetric flask. The NaCl was then allowed to dissolve using an inverting technique to aid the process. Once fully dissolved the solutions were kept at ambient conditions until further use.

### Preparation of Gellan Sols:

Gellan solutions were prepared by dissolving powdered polymer into water/NaCl solution so that the final concentrations post-processing were equal to 0.9% and 1.8% (w/v). In brief, gellan powder was weighed out (4.5 g, 9.0 g) and added to 450 ml of deionised water. The mixture was allowed to heat to 95° C. under agitation, allowing the polymer to dissolve. Once fully dissolved, 25 ml of NaCl stock solution (0.2 M) was added to the solution resulting in a 10 mM concentration post-processing. The sol was then allowed to reach thermal equilibrium at 95° C. before processing.

### Processing of Gellan MS:

MS were prepared using a jacketed pin mill set to 20° C. Gellan sols were pumped using a peristaltic pump into the pin mill at 3 ml/min so that it entered the processing chamber at 40° C. Prior to entry using a syringe and syringe pump, water was pumped into the gellan stream (at a rate of 0.16 ml/min) so that they impinged, diluting the gellan sol to the final concentrations (0.9 and 1.8% (w/v), 10 mM NaCl). The mixture was then cooled under shear (100, 500, 1000 and 2000 rpm) as it passed through the milling unit. On exiting, at 20° C., the gel was packaged and stored at 4° C. until further testing.

### Material Analysis:

### Rheometry:

A rheometer (TA, AR-G2) equipped with a sandblasted parallel plate (40 mm diameter, 1 mm gap height) was used to test all samples, at 20° C.

### Amplitude Sweeps:

Amplitude sweeps were obtained in strain controlled mode over a range of 0.1 to 100.0%. Samples were loaded into the instrument and upper geometry lowered. Once trimmed, the sample was left to equilibrate at 20° C. prior to testing. Measurements were obtained at 1 Hz in a logarithmic fashion.

### Flow Profiles:

Viscosity profiles for the samples were obtained using a continuous ramp. Samples were loaded into the instrument and upper geometry lowered. Once trimmed, the sample was left to equilibrate at 20° C. prior to testing. Increasing shear was applied to the sample in rate controlled mode, between 0.1 and 600 over a 3-minute ramp, with data points obtained in a logarithmic fashion.

### Results:

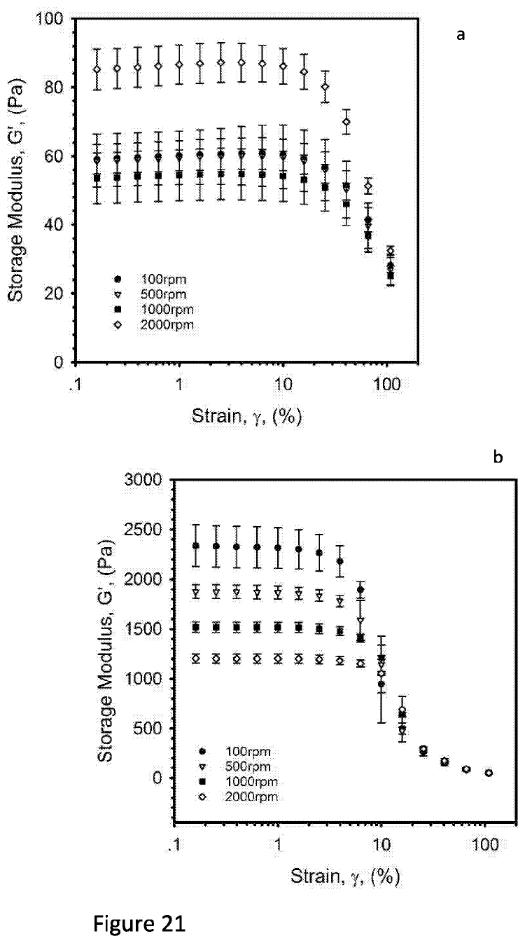

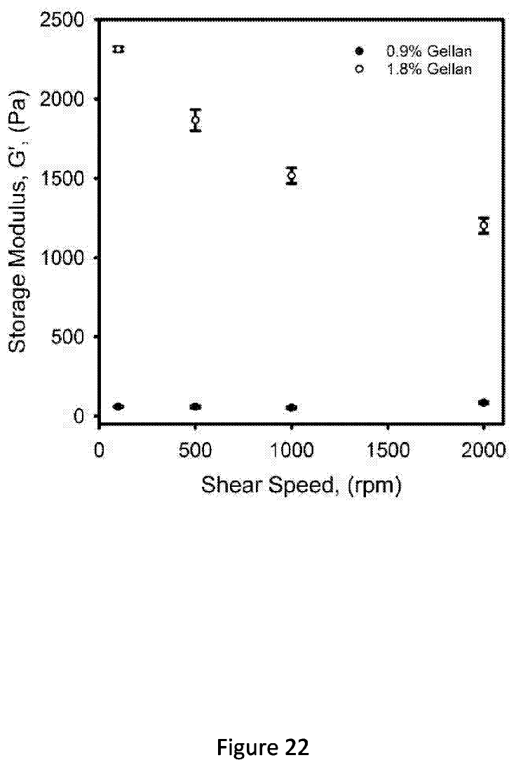

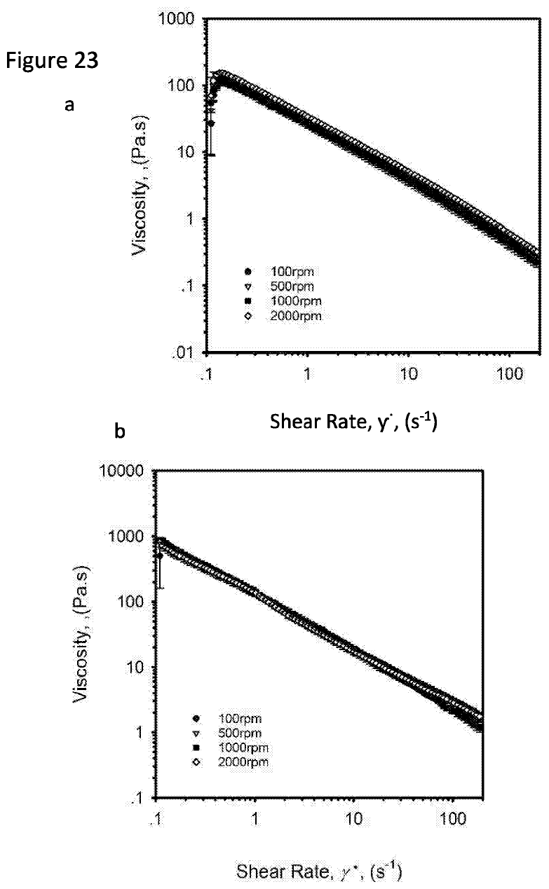

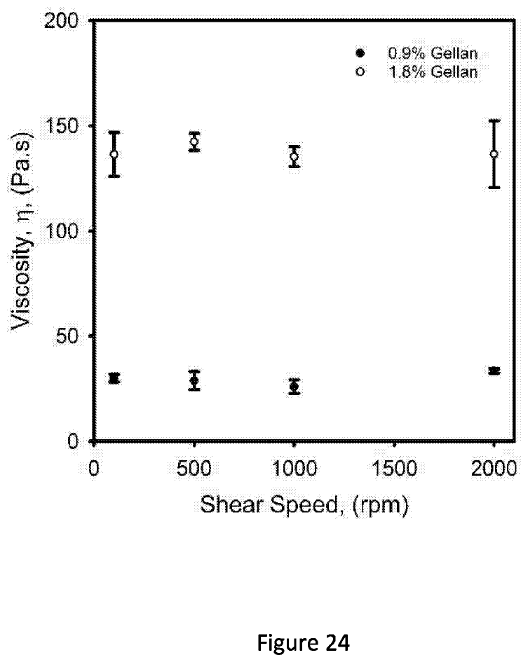

Small deformation rheology: See FIGS. 21 and 22
Large deformation rheology: See FIGS. 23 and 24

### Discussion:

The degree of shear applied throughout the sol-gel transition of the gellan biopolymer was studied at two concentrations, 0.9% (w/v) and 1.8% (w/v). At the lower polymer concentration both degree of elasticity, as defined by G′, and viscosity was independent of the degree of shear experienced throughout the gelling profile. In all cases, the resultant material exhibited shear thinning over large deformations and solid-like behaviours at rest, however, the magnitude of such observations did not change (FIGS. 2 and 4). The same was found for the viscosity profiles of the 1.8% (w/v) system, where, although elevated viscosities were observed in comparison to the 0.9% (w/v) systems, they were independent of the shear applied during processing. However, the elastic nature of the systems at rest did show a dependency, with increasing shear reducing the final storage modulus (G′). It is believed that increasing the mixing applied during the gelation process directly effects the microstructure of the individual particles, with increased levels of confinement prevent the growth of more rigid particles. As a result, the particles are more deformable, with lower G′.

### Conclusions:

In summary, changing the mixing speed during processing does not play a large role in the resultant material properties of the microgel suspensions with low polymer concentrations. As such, a wide range of processing shears can be applied without altering the final properties of the eye drop formulation. However, for the higher concentrations, used for “cream-like” spreadable systems, sheared processing plays more of an important role in the degree of solid-like behaviour at rest. In these cases, the degree of elasticity can be manipulated for the intended use.

### Further Experimental Information: Fluid gel Formation and Properties

### Aims:

To show the ability to prepare fluid gels form a variety of starting biopolymers (Gellan, Kappa-carrageenan, Alginate, Agar) with different gelation mechanisms at varying concentrations.
To demonstrate the material properties of these fluid gels.

### Materials and Methods:

### Preparation of Fluid Gels:

Fluid gels were prepared as follows:

### Gellan (Thermal Gelation):

Addition of gellan powder to water with 5% PBS to form a 0.5 to 2% polymer solution.
Heating the solution above the gelling point.
Adding crosslinker (sodium chloride (10 mM final concentration))
Cooling the solution through the gelling point (ca. 38° C.) whilst constantly shearing.

### Kappa-Carrageenan (Thermal Gelation):

Addition of gellan powder to water with 5% PBS to form a 0.5 to 2% polymer solution.
Heating the solution above the gelling point.
Adding crosslinker (potassium chloride (10 mM final concentration))
Cooling the solution through the gelling point (ca. 40° C.) whilst constantly shearing.

### Alginate (Ionotropic Gelation):

Addition of alginate powder to water with 5% PBS to form a 0.5 to 1% polymer solution.
Fully hydrating the polymer. (if aided by heat, allow to cool back to room temperature)
Adding crosslinking agent (calcium chloride added (10 mM final concentration))
slowly using a syringe and needle whilst constantly shearing.
   Agar (Thermal Gelation with Hysteresis (Melting and Gelling Point not the Same)):
Addition of agar powder to water with 5% PBS to form a 0.5 to 2% polymer solution.
Heating the solution above the gelling point (above 90° C.).
Adding crosslinker (sodium chloride added (10 mM final concentration))
Cooling the solution through the gelling point (ca. 36° C.) whilst constantly shearing.

### Rheological Testing:

A rheometer equipped with a serrated parallel plate (40 mm diameter, 1 mm gap height) was used to test all samples, at 20° C.

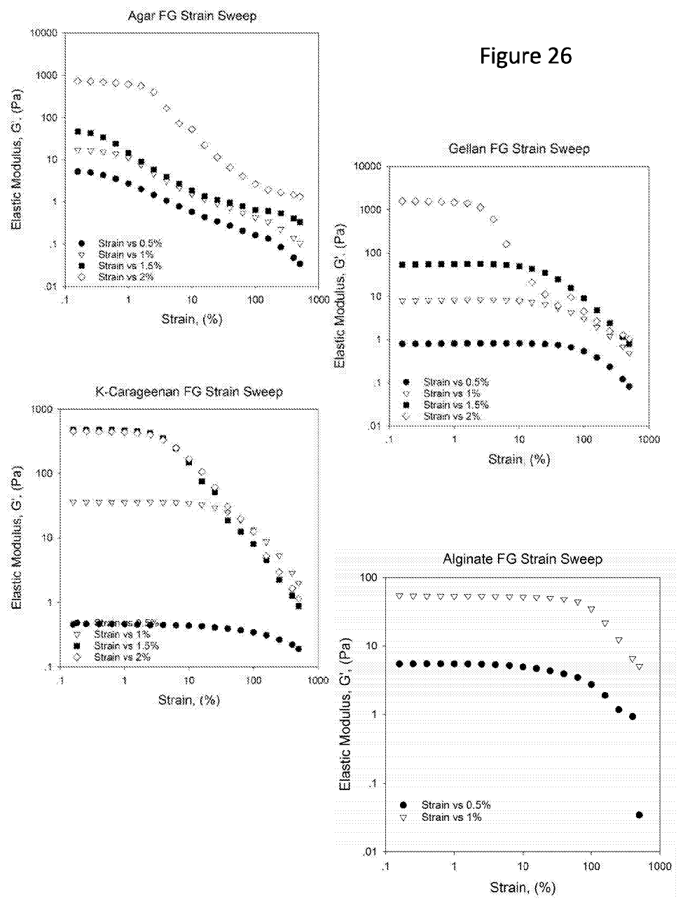

### Amplitude Sweeps (FIG. 26):

Amplitude sweeps were obtained in strain-controlled mode over a range of 0.1 to 500.0%.
Once loaded, the samples were left to equilibrate at 20° C. prior to testing.
Measurements were obtained at 1 Hz in a logarithmic fashion.

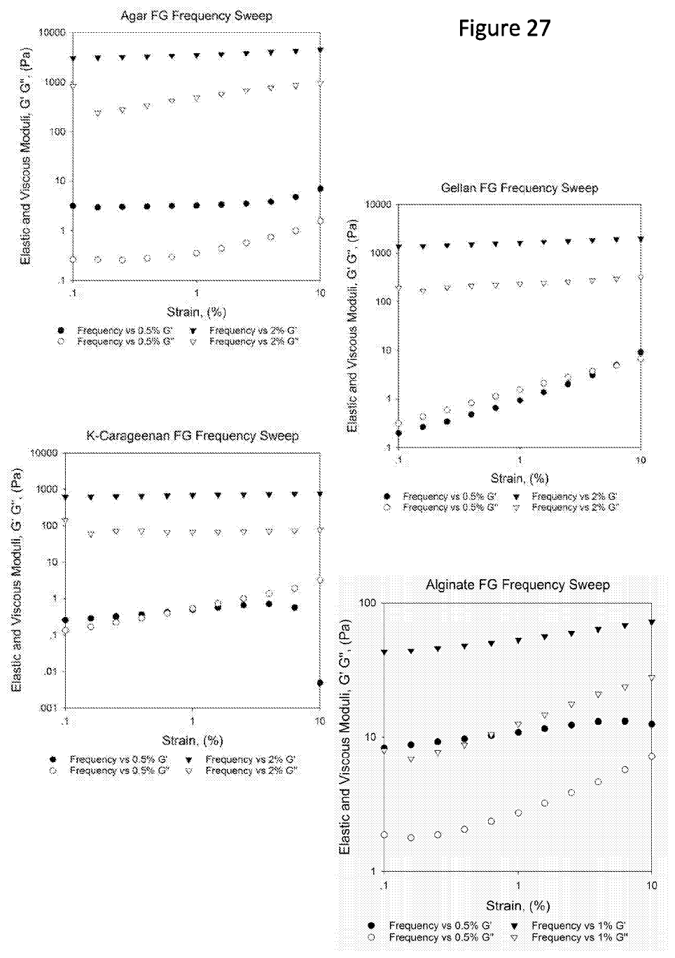

### Frequency Sweeps (FIG. 27):

Frequency sweeps were conducted at a strain within the linear viscoelastic region of the amplitude sweep.
Once loaded, the sample were left to equilibrate at 20° C. prior to testing.
Samples were tested between 0.01 and 10 Hz, in a logarithmic fashion.

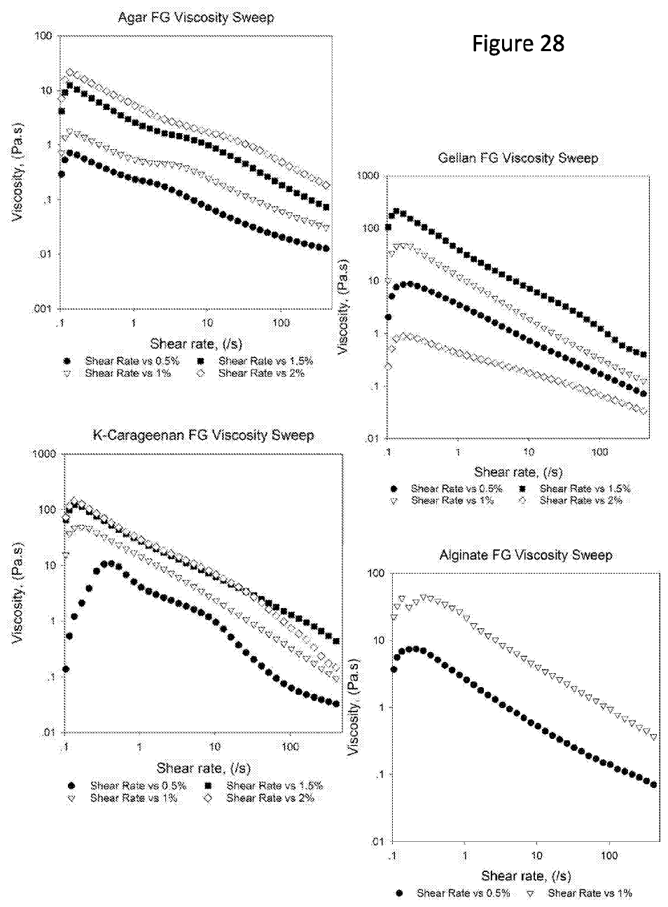

### Flow Profiles (FIG. 28):

Viscosity profiles for the samples were obtained using a continuous ramp.
Samples were loaded into the instrument and left to equilibrate at 20° C. prior to testing.
Increasing shear was applied to the sample in rate-controlled mode, between 0.1 and 600 over a 3-minute ramp, with data points obtained in a logarithmic fashion.

### Results:

FIGS. 26-28 shows amplitude sweep, frequency sweep and viscosity sweep data obtained for agar, gellan, kappa carageenan and alginate.

### Discussion:

The data obtained showed that:
All systems showed mechanical properties typically associated with fluid gels: weak solid-like behaviour at rest (frequency sweeps); breakdown of the solid behaviour with applied stain, yielding (amplitude sweeps); and shear thinning behaviour (viscosity profiles).
The data shows that fluid gels can be made using a range of different biopolymers including gellan.
Data demonstrates that various gelation mechanisms can be used to fabricate the fluid gels:
   a. thermally driven processes (gellan, agar, K-carrageenan);
   b. lonotropic gelation—gelation through crosslinking via ionic species (no heat) (alginate).
Furthermore, in the case of thermally driven processes it shows that various ionic species can be used (Na⁺ and K⁺).
Mechanical responses of the gels can all be categorised in remits of the material properties previously reported within the patent. (either within the outer limits, or within the narrowed window that was stated as more optimum).

Conclusions:

In summary, this data shows that it is possible to fabricate fluid gels from a range of polymers. This has been demonstrated using a range of biopolymers which have different gelation mechanisms: thermal, thermal with hysteresis, ionotropic and free radical. This wide range of examples suggests a blanket means to fabricate the fluid gels such that any biopolymer solution equal to or above the critical gelling concentration which is forced through its sol-gel transition (thermally, ionotropically, radical induced . . . ) under appropriate shear to prevent a full continuous gel network from forming can be used to fabricate fluid gels.

### Further Experimental Information—Release of Actives for Various Fluid Gels

### Aims:

To show the ability to release various actives across a range of different indications (anti-fibrotic, anti-infective, pain relief, anti-inflammatory, ECM modifying, basement membrane modifying and pro-fibrotic) from the fluid gel matrices fabricated form a variety of starting polymers (particularly Gellan and Alginate).

### Materials and Methods:

### Preparation of Fluid Gels:

Fluid Gels were Prepared by:

### Gellan (Thermal Gelation):

Addition of gellan powder to water with 5% PBS to form a 0.5 to 2% polymer solution.
Heating the solution above the gelling point.
Adding crosslinker (sodium chloride (10 mM final concentration))
Cooling the solution through the gelling point (ca. 38° C.) whilst constantly shearing.

### Alginate (Ionotropic Gelation):

Addition of alginate powder to water with 5% PBS to form a 0.5 to 1% polymer solution.
Fully hydrating the polymer. (if aided by heat, allow to cool back to room temperature)
Adding crosslinking agent (calcium chloride added (10 mM final concentration)) slowly using a syringe and needle whilst constantly shearing.

### Preparation of Active:

Aliquot active: Penicillin-streptomycin (0.1 ml), dexamethasone (50 mg), proteinase K (10 mg), ibuprofen (200 mg), dextran (300 mg), dextran blue (100 mg), vancomycin (50 mg), Galacorin™ (decorin) (2.4 mg/ml)
Add active to PBS to make a total volume of 1 ml.
Mix well on a vortex mixer until dissolved

### Preparation of Active Loaded Gels:

Add 0.9 ml of gel to an Eppendorf.
To each gel add 0.1 ml of active in PBS.
Mix well using a vortex mixer.
Refrigerate for 24 hours before testing.

### Determination of Standard Curves:

Standard concentrations of active in PBS were prepared.
Standards were pipetted into a quartz cuvette (1 mm pathlength).
UV/vis spectroscopy was used to measure absorbance between 200 and 700 nm wavelength.
Curves were plotted and used to determine standard curves used to determine concentrations.
   (in the case of Galacorin™ an off the shelf ELISA kit was used per kit instructions to determine decorin concentrations)

### Release Assay:

0.5 ml PBS was added to the well of a 24 well plate.
The PBS was incubated at 37° C. to equilibrate.
0.1 ml of the fluid gel containing active was placed in a trans-well insert.
The trans-well insert was placed into a well containing PBS.
After a given time period the trans-well insert was removed and placed in a fresh well of PBS.
The release media was then removed and analysed using UV/vis spectroscopy.
Concentrations were derived from the standard curves and cumulative release plotted as a function of time.

### Results:

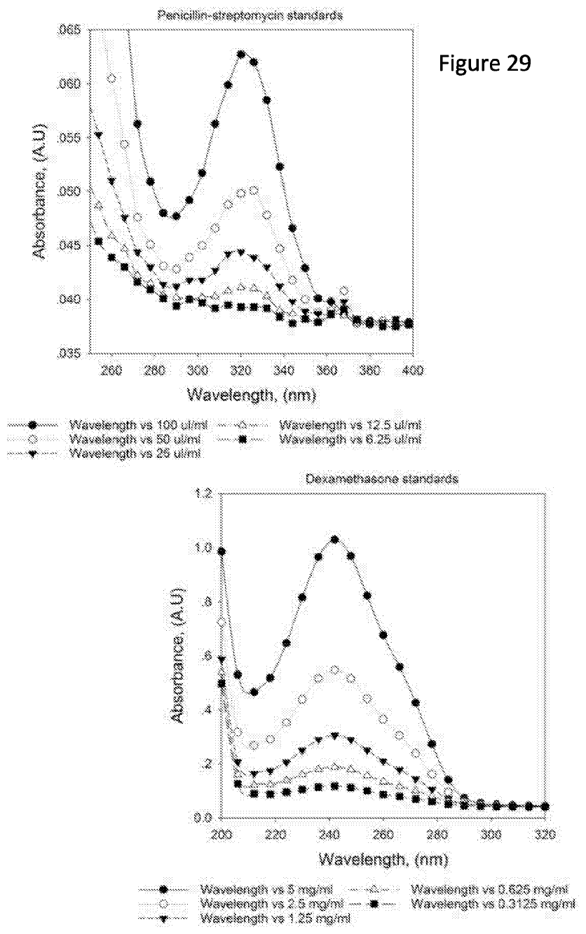

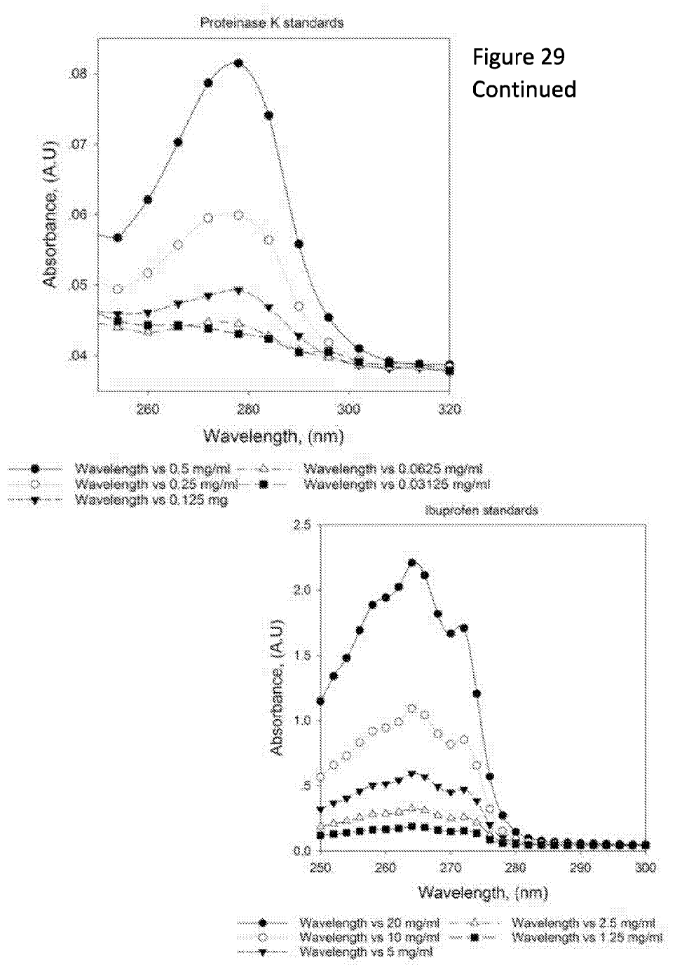

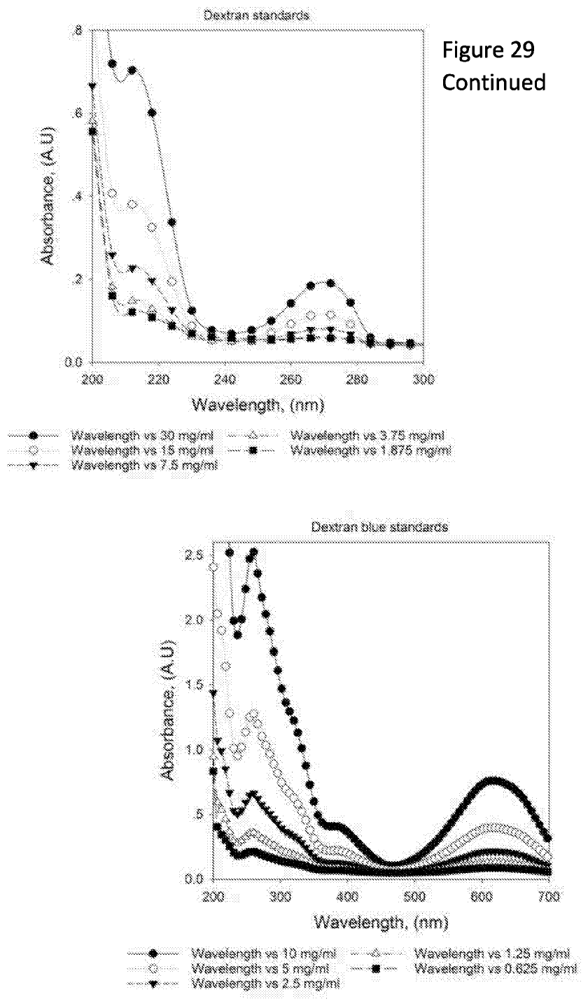

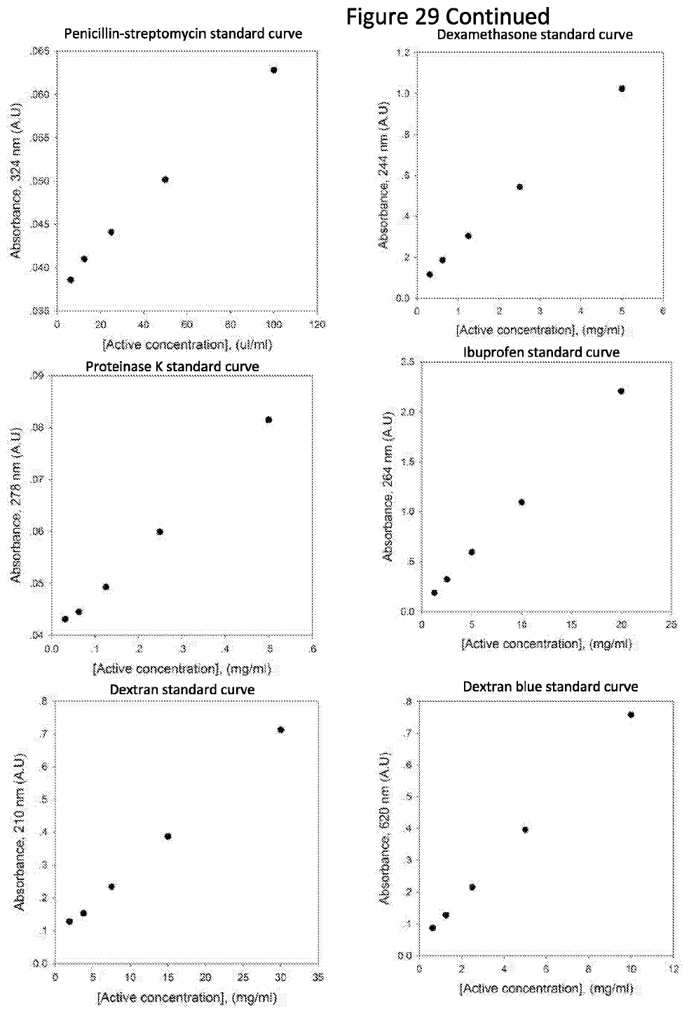

FIG. 29 illustrate standard curves obtained in respect of shear-thinning hydrogel compositions in accordance with the invention incorporating the following active agents: penicillin-streptomycin; dexamethasone; proteinase K; ibuprofen; dextran; and dextran blue.

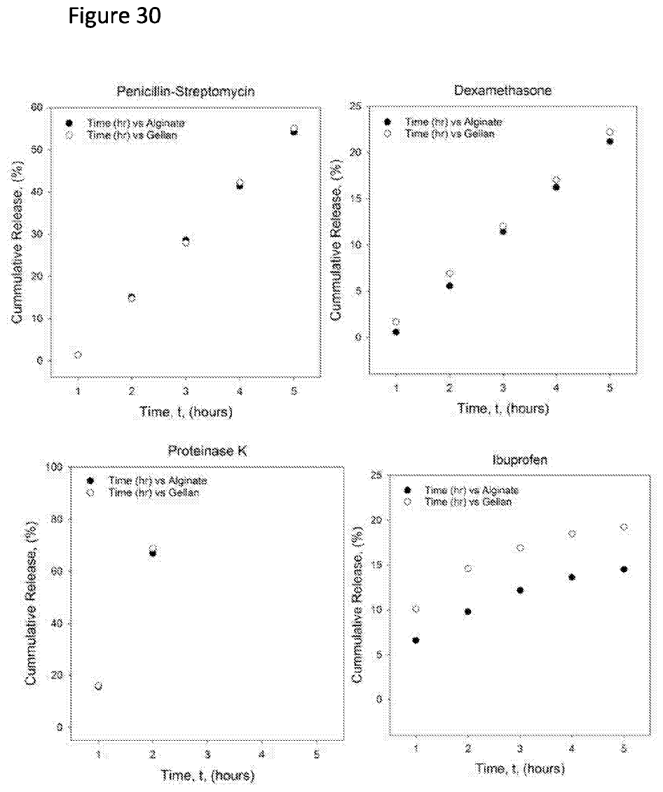

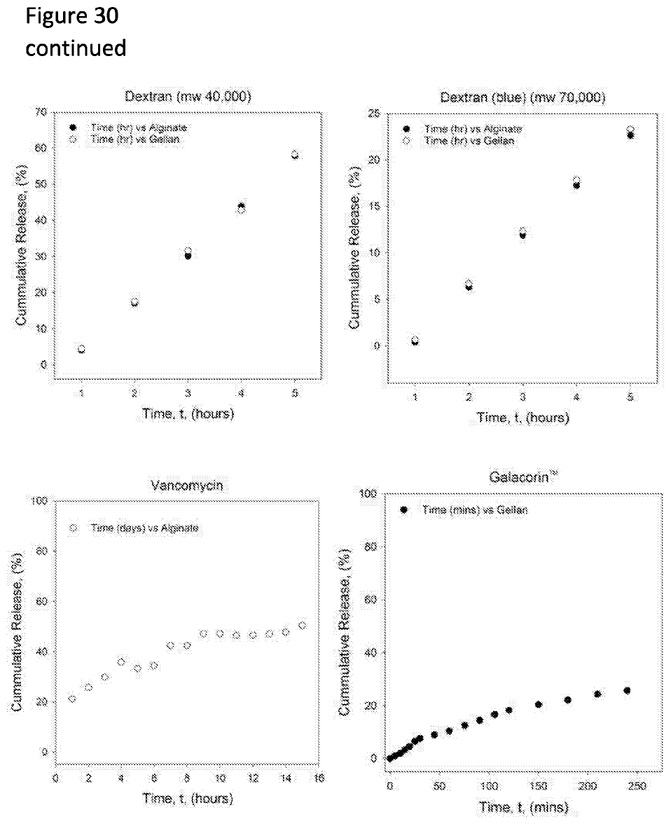

FIG. 30 illustrate curves obtained in respect of shear-thinning hydrogel compositions in accordance with the invention incorporating the following active agents: penicillin-streptomycin; dexamethasone; proteinase K; ibuprofen; dextran; and dextran blue.

### Discussion:

Data obtained in the results above showed that:
Loading and release of the actives could be achieved from all fluid gel matrices irrespective of biopolymer (gellan, alginate) or polymer mechanism.
Loading and release of the actives could be achieved from all fluid gel matrices irrespective of gelation mechanism: thermally gelled, ionotropically gelled or radical induced gelation.
Fluid gels could release both small molecules (Ibuprofen, dexamethasone, Penicillin-streptomycin) and macromolecules (Dextran, Dextran blue, Proteinase K, Galacorin™ (decorin)).
Fluid gels can be used for controlled delivery to the following indications:
   anti-fibrotic—Galacroin™ (decorin), Dextran(s)
   anti-infective—Vancomycin, Penicillin-streptomycin
   pain relief—ibuprofen
   anti-inflammatory—ibuprofen, dexamethasone
   ECM modification—Proteinase K
   basement membrane modification—proteinase K
   pro-fibrotic—Dextran

### Conclusions:

Shear-thinning hydrogel compositions (fluid gels) in accordance with the invention made from a range of polymers and using various gelation techniques can be used to deliver a wide range of therapeutics, both large and small molecules. This suggests that a wide range of therapeutics could be delivered in these cases. The suitability of a therapeutic agent for delivery by this method does not appear to be governed by the size of the molecule or type (protein or polysaccharide) but (in this study) depends upon the agent being water soluble. This has been shown for an exemplar of active agents suitable for use in the treatment of a wide range of indications.

Further Experimental Information: In Vitro Release and Action of Anti-Infective Molecules from Gel Compositions of the Invention

### Aims:

To show the potency of anti-infective therapeutics (exemplified by vancomycin and penicillin-streptomycin) on release from shear-thinning hydrogel compositions in accordance with the invention.

### Materials and Methods:

### Preparation of Fluid Gels:

Fluid Gels were Prepared by:

### Gellan

Addition of gellan powder to water with 5% PBS to form a 1% polymer solution.
Heating the solution above the gelling point.
Adding crosslinker (sodium chloride added (10 mM final concentration))
Cooling the solution through the gelling point whilst constantly shearing.

### Alginate

Addition of alginate powder to water with 5% PBS to form a 0.5% polymer solution.
Fully hydrating the polymer. (if aided by heat, allow to cool back to room temperature)
Adding crosslinking agent (calcium chloride added (10 mM final concentration)) slowly using a syringe and needle whilst constantly shearing.

### Preparation of Active:

Aliquot active: penicillin-streptomycin (100 ml) and vancomycin (50 mg).
Add active to PBS to make a total volume of 1 ml.
Mix well on a vortex mixer until dissolved

### Preparation of Active Loaded Gels:

Add 0.9 ml of gel to an Eppendorf.
To each gel add 0.1 ml of active in PBS.
Mix well using a vortex mixer.
Refrigerate for 24 hours before testing.

### Preparation of Microbes:

Prepare TSA plates by dissolving TSA in water, sterilising via autoclave and casting into 90 mm petri dishes.
Allow plates to cool.
Culture microbes (E. coli and S. aureus) and plate.
Allow microbes to form a “lawn”.
Bore a hole into the gel and remove to provide a well.

### Zone of Inhibition Assay:

Add 0.25 ml of fluid gel containing active to the well of each plate.
Add anti-infective in PBS to the control plate.
Cover plate and incubate 24 hours for pencillin-streptomycin, up to 14 hours for vancomycin.
Measure area where microbe culture has been removed.

### Results:

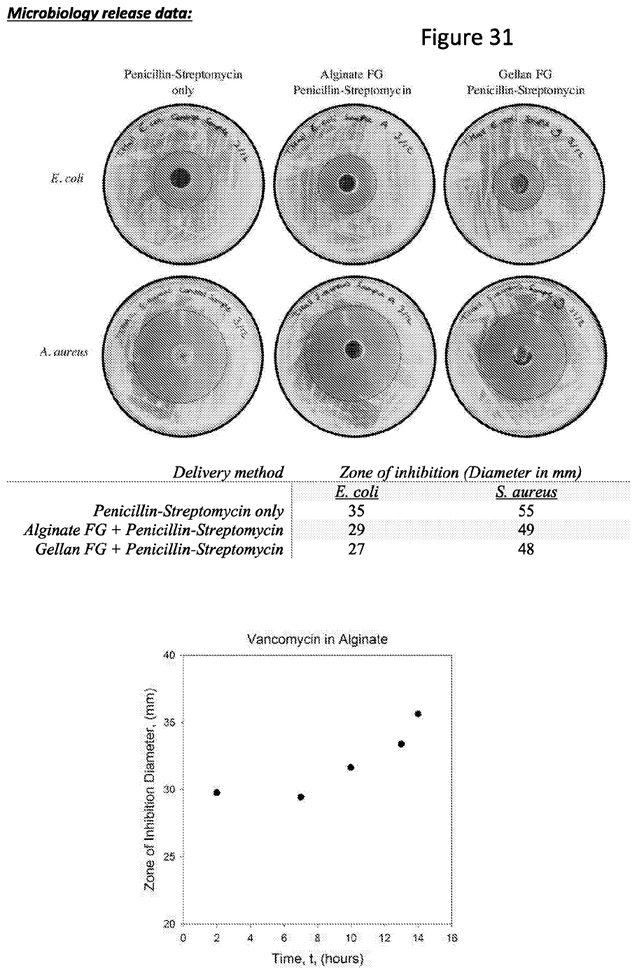

FIG. 31 shows photographs illustrating the results of zone of inhibition assays using shear-thinning hydrogel compositions in accordance with the invention comprising the polymers alginate or gellan, in combination with an anti-infective agent (penicillin-streptomycin). These results demonstrate effectiveness in respect of E. coli and S. aureus. A summary of the results is also provided in the accompanying Table.

FIG. 31 also includes a graph illustrating the results of zone of inhibition assays using shear-thinning hydrogel compositions in accordance with the invention comprising alginate in combination with an alternative anti-infective agent (vancomycin). Anti-microbial effectiveness was tested against MRSA.

### Discussion

Data Showed that:
Release and activity of the anti-infectives occurred across all systems irrespective of polymer type, gelling mechanism or active.

### Conclusions:

Fluid gels made from a range of polymers and using various gelation techniques can be used to deliver anti-infectives without compromising their activity. These results illustrate the suitability of shear-thinning gel compositions of the invention to deliver a variety of anti-infective agents for use in therapies requiring such agents.

Further Experimental Information: In Vitro Action of Proteinase K Released from Fluid Gels

### Aims:

To show that the extracellular matrix remodelling agent proteinase K retains biological activity after release from shear-thinning hydrogel compositions in accordance with the invention.

### Materials and Methods:

### Preparation of Fluid Gels:

Fluid Gels were Prepared by:

### Gellan

Addition of gellan powder to water with 5% PBS to form a 1% polymer solution.
Heating the solution above the gelling point.
Adding crosslinker (sodium chloride added (10 mM final concentration))
Cooling the solution through the gelling point whilst constantly shearing.

### Alginate

Addition of alginate powder to water with 5% PBS to form a 0.5% polymer solution.
Fully hydrating the polymer. (if aided by heat, allow to cool back to room temperature)
Adding crosslinking agent (calcium chloride added (10 mM final concentration)) slowly using a syringe and needle whilst constantly shearing.

### Preparation of Active:

Aliquot active: proteinase K (10 mg)
Add active to PBS to make a total volume of 1 ml.
Mix well on a vortex mixer until dissolved

### Preparation of Active Loaded Gels:

Add 0.9 ml of gel to an Eppendorf.
To each gel add 0.1 ml of active in PBS.
Mix well using a vortex mixer.
Refrigerate for 24 hours before testing.

### Matrix Breakdown Assay:

0.5 ml of fibrin gel (8.5 mg/ml) was formed in the wells of a 24 well plate.
0.5 ml of PBS was added to each well.
0.1 ml of gel with active was added to a trans-well insert and placed above the fibrin gel.
Samples were incubated at 60° C. to activate the proteinase K.
Images were taken at various timepoints and compared to the control fibrin gel (pbs only) and fibrin gel with proteinase k added without a fluid gel carrier.

### Results:

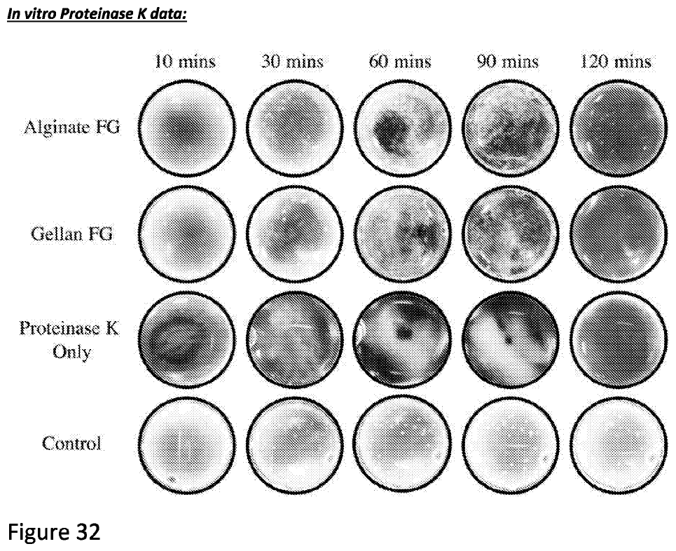

FIG. 32 shows photographs demonstrating breakdown over time of the exemplary ECM molecule fibrin (shown as a white gel in the photographs) under the action of the active agent proteinase K released from alginate or gellan shear-thinning hydrogel compositions in accordance with the invention.

### Discussion

Data Showed that:
Break down of the fibrin gel occurred in all cases apart from the control group.
Fibrin gel break down was faster in the proteinase K only group.
Active is still potent after being released from the gels.

### Conclusions:

Shear-thinning hydrogel compositions in accordance with the invention were able to release an extracellular remodelling active agent (proteinase K), which retained its' ability to modify ECM. This demonstrates the ability of the compositions of the invention to deliver therapeutic molecules of this sort.

Further Experimental Information: In Vitro Action of Galacorin™ (Decorin) Formulated in Gellan Shear-Thinning Hydrogel Compositions in Accordance with the Invention

### Aims:

To show the potency of anti-fibrotic therapeutics (exemplified by the commercially available human recombinant decorin product, Galacorin™) on release from shear-thinning hydrogel compositions of the invention.

### Materials and Methods:

### Preparation of Fluid Gels:

Fluid Gels were Prepared by:

### Gellan

Addition of gellan powder to water with 5% PBS to form a 1% polymer solution.
Heating the solution above the gelling point.
Adding crosslinker (sodium chloride added (10 mM final concentration))
Cooling the solution to 40° C. under constant shear and adding Galacorin™ (final concentration of 240 ug/ml)
Continue cooling through the gelling point whilst constantly shearing.

### Collagen Fibrillogenesis Assay:

Prepare diluent and reaction buffers by mixing sodium phosphate and sodium chloride and adjusting the pH to 7.4.
Add sample (Gellan fluid gel +Galacorin™ Galacorin only and Gellan only) to a 96 well plate and serial dilute using diluent buffer across the rows of the plate.
On ice, prepare collagen by mixing with cold water to a concentration of 0.8 mg/ml.
Add collagen to all wells.
Add reaction buffer to all wells and mix.
Incubate at 37° C. for 2 hours.
Read using a plate reader at 405 nm

### Results:

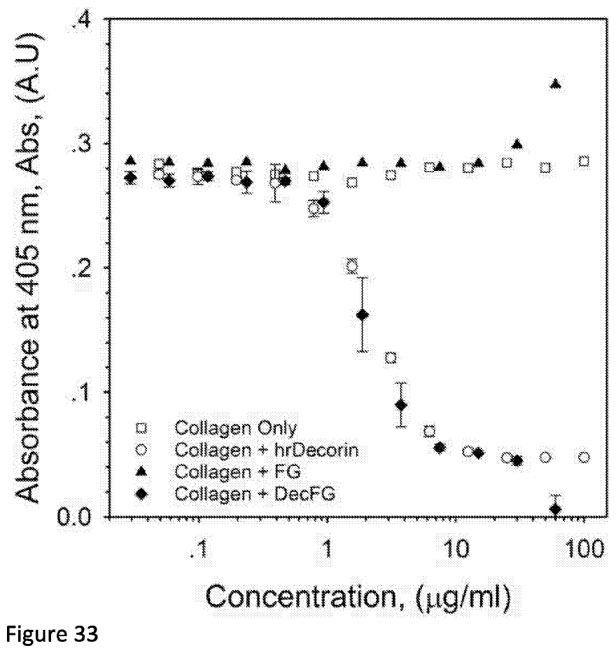

FIG. 33 is a graph illustrating the results of this study, and comparing absorbance at 405 nm (y-axis) for collagen alone (“collagen only”), or collagen incubated with decorin alone “hrDecorin”) or with increasing concentrations of gellan fluid gel shear-thinning hydrogel compositions of the invention with (“DecFG”) or without (“FG”) human recombinant decorin (Galacorin™)

### Discussion:

Data Showed that:
The gellan fluid gel had no effect on collagen fibrillogenesis in comparison to the collagen only control.
Galacorin™ either in or out of the gellan fluid gel had the same effect on the collagen fibrillogenesis.

### Conclusions:

Collagen fibrillogenesis can be used an indicative assay for scarring. As such, high absorbances, as a consequence of poor collagen microstructure (comparable to scarred tissue), indicates that a test composition has no effect on scarring. In contrast, a reduction in the absorbance indicates more orderly collagen microstructure, which is comparable to better healing in vivo. Galacorin™ (decorin) loaded into gellan fluid gel has been demonstrated here to have the same effect as Galacorin™ added straight to the collagen. As such this demonstrates in vitro that gellan shear-thinning hydrogel compositions in accordance with the invention can release the therapeutic anti-fibrotic agent, and that it retains its activity upon release.

Further Experimental Information: In Vivo Action of Galacorin™ (Decorin) from Gellan Shear-Thinning Hydrogel Compositions of the Invention

### Aims:

To show the potency of an anti-fibrotic therapeutic (Galacorin™) on release from the fluid gel carrier in an in vivo mouse model of microbial keratitis.

### Materials and Methods:

### Preparation of Fluid Gels:

Fluid Gels were Prepared by:

### Gellan

Addition of gellan powder to water with 5% PBS to form a 1% polymer solution.
Heating the solution above the gelling point.
Adding crosslinker (sodium chloride added (10 mM final concentration))
Cooling the solution to 40° C. under constant shear and adding Galacorin™ (final concentration of 240 ug/ml)
Continue cooling through the gelling point whilst constantly shearing.

### Mouse Model:

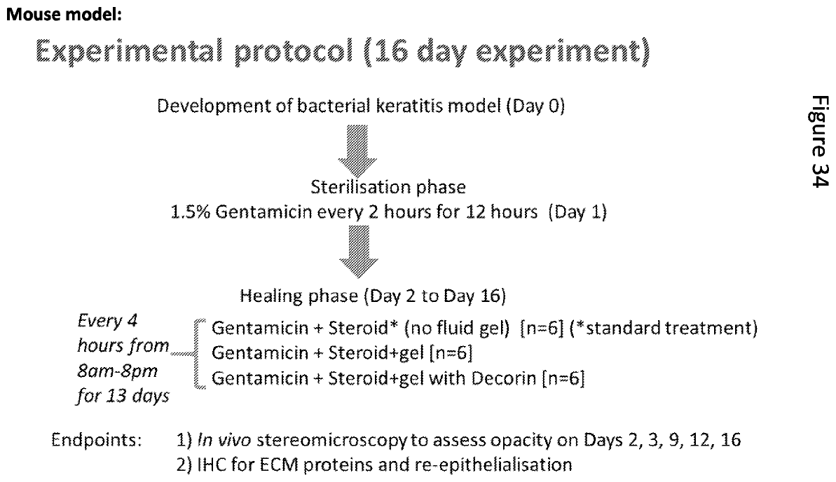

A representation of the mouse model used in this study is set out in FIG. 34. Briefly, this shows that the model progresses through three phases: the development of the bacterial keratitis model, the sterilisation phase, and the healing phase. The endpoints used are in vivo stereomicroscopy (used to assess opacity on days 2, 3, 9, 12, and 16) and immunohistochemistry analysis of tissue sections to investigate expression of ECM proteins and the extent of re-epithelialisation.

### Opacity Quantification:

Two ophthalmologists acting as masked independent observers analyzed all photographs in the same randomized order (the order provided by an independent statistician).
The area of opacification was measured using Fiji, an open source image-processing package based on imageJ.
Measurements, in mm², were plotted using ggplot2 in R, and loess smoothers fit to the chronological series for each assessor.

### Tissue Processing and IHC for Re-Epithelialization, αSMA, Lam and FN:

Eyes were fixed in 4% PFA in PBS.
Eyes were then rapidly frozen in OCT and sectioned in the parasagittal plane −22° C. at a thickness of 15 μm.
Sections were mounted on positively charged glass slides (Superfrost plus; Fisher Scientific, Pittsburgh, Pa., USA).
Central sections (in the optic nerve plane) were used for all IHC studies.
Sections were left to thaw for 30 minutes before washing in PBS followed permeabilization with 0.1% Triton X-100 (Sigma).
Nonspecific antibody binding sites in tissue sections were blocked with 0.5% BSA, 0.3% Tween-20 and 15% normal goat serum.
Primary antibody αSMA, Laminin and fibronectin (dilution of 1:200) was added, followed by washing in PBS.
Incubation for 1 hour at RT with secondary antibody (Goat anti-mouse Alexa Flour 488 1:500, Goat anti-mouse Alexa Flour 594 1:500) was then undertaken.
Sections were then washed in PBS and mounted in Vectorshield mounting medium containing DAPI.
Control tissue sections incubated with secondary antibody alone were all negatively stained (not shown).

### IHC Imaging and Quantification:

IHC staining was quantified by measuring pixel intensity.
The region of interest used for quantitation of ECM IR was defined by a region of interest which was same prescribed size for all eyes/treatments within the stroma, each stroma had a total of 30 individual intensity measurements (regions of interest) taken to cover the whole area of the stroma.
Extracellular matrix deposition was quantified within these defined regions of interests within the stroma and the percentage of immunofluorescent pixels above a standardized background threshold was calculated using ImageJ software.
For each antibody, the threshold level of brightness in the area of stroma was set using intact untreated eye sections to define the reference level for test group analysis of pixel intensity.

### Results:

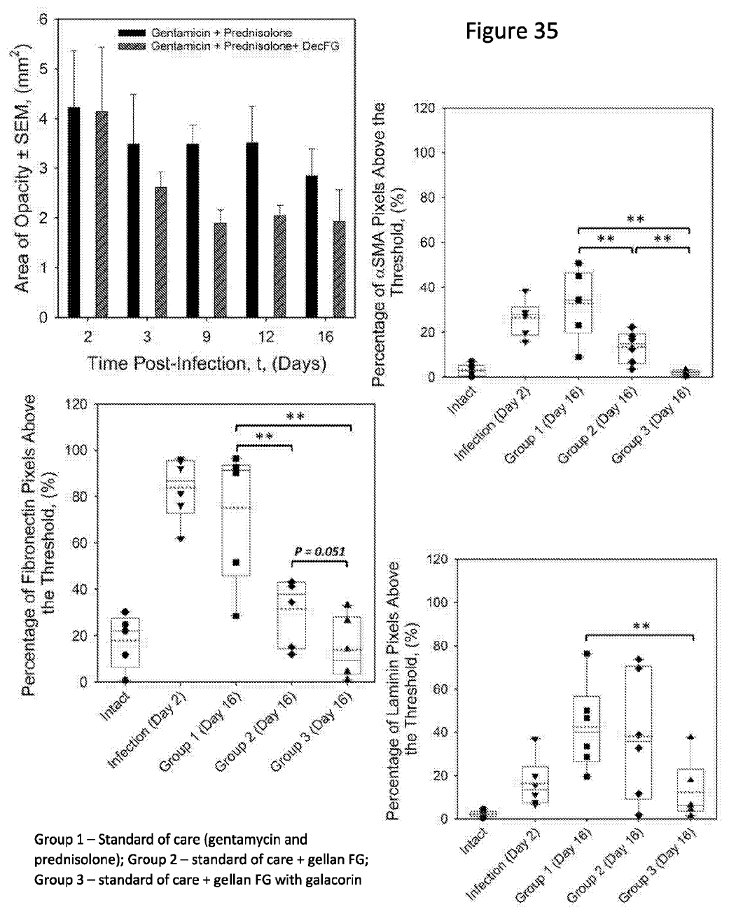

Results of the study are illustrated in FIG. 35, which sets out graphs showing the area of opacity associated with the different treatments at different timepoints, the percentage of α-smooth muscle actin pixels above the threshold value for the various control and treatment groups investigated, the percentage of fibronectin pixels above the threshold value for the various control and treatment groups investigated, and the percentage of laminin pixels above the threshold value for the various control and treatment groups investigated.

### Discussion:

Data Showed that:
A gellan shear-thinning hydrogel (fluid gel) composition in accordance with the invention comprising Galacorin™ reduced the area of opacity over the 16 days in comparison to the standard of care (Genatmicin+prednisolone only).
A gellan shear-thinning hydrogel (fluid gel) composition in accordance with the invention comprising Galacorin™ significantly reduced all three tested markers for fibrosis in comparison to the standard of care alone.

### Conclusions:

Application of the anti-fibrotic agent within a gellan shear-thinning hydrogel (fluid gel) composition in accordance with the invention reduced scarring in vivo. This reduction in scarring was demonstrated by both reduced areas of opacity (scar formation) and reduced expression of markers typically associated with fibrosis and scarring.

Further Experimental Information: Shear-Thinning Hydrogel Compositions in Accordance with the Invention Reduce Intraocular Pressure

### Aim

The study aimed to investigate the potential of shear-thinning hydrogel compositions without active agents to reduce intraocular pressure in hypertensive rats (an animal model of glaucoma).

### Materials and Methods

Hypertension was induced in rats by twice weekly intracameral injections of TGF-1. Net change in intraocular pressures were determined in hypertensive rats (n=9) without treatment and in rats which receiving twice daily eye drop formulations of a shear-thinning hydrogel composition of the invention made with gellan.

### Results

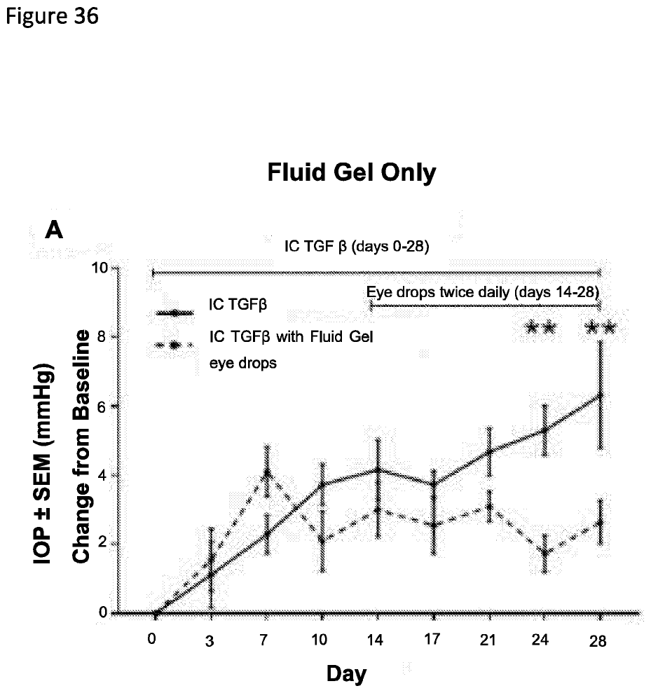

The results are shown in FIG. 36, in which treated rats are shown in dashed line, and non-treated controls with a solid black line. Results were analysed with two-way ANOVA, with Sidak multiple comparisons tests and demonstrated that the composition of the invention (p<0.05) significantly reduced intraocular pressure in ocular hypertensive rats by D28 compared to controls.

### Conclusions

The results achieved demonstrate that shear-thinning hydrogel formulations of the invention are able to reduce intraocular hypertension (indicating an ability to prevent or treat glaucoma). Surprisingly, this activity was observed even when the compositions were formulated without active agents.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCES

1. Oliva M S, Schottman T, Gulati M. Turning the tide of corneal blindness. Indian journal of ophthalmology. 2012; 60(5):423.
2. Konda N, Motukupally S R, Garg P, Sharma S, Ali M H, Willcox M D. Microbial

Analyses of Contact Lens-Associated Microbial Keratitis. Optometry and Vision Science. 2014; 91(1):47-53.
3. Stapleton F, Dart J, Seal D, Matheson M. Epidemiology of Pseudomonas aeruginosa keratitis in contact lens wearers. Epidemiology & Infection. 1995; 114(3):395-402.
4. Wu Y T-Y, Willcox M, Zhu H, Stapleton F. Contact lens hygiene compliance and lens case contamination: A review. Contact Lens and Anterior Eye. 2015; 38(5):307-16.
5. O'brien T. Management of bacterial keratitis: beyond exorcism towards consideration of organism and host factors. Eye. 2003; 17(8):957.
6. Willcox M D. Pseudomonas aeruginosa infection and inflammation during contact lens wear: a review. Optometry and Vision Science. 2007; 84(4):273-8.
7. Tandon A, Tovey J C, Sharma A, Gupta R, Mohan R R. Role of transforming growth factor Beta in corneal function, biology and pathology. Current molecular medicine. 2010; 10(6):565-78.
8. Allan B D, Dart J K. Strategies for the management of microbial keratitis. The British Journal of Ophthalmology. 1995; 79(8):777-86.
9. Gokhale N S. Medical management approach to infectious keratitis. Indian Journal of Ophthalmology. 2008; 56(3):215-20.
10. Ralph R A. Tetracyclines and the Treatment of Corneal Stromal Ulceration: A Review. Cornea. 2000; 19(3):274-7.
11. Shoham A, Hadziahmetovic M, Dunaief J L, Mydlarski M B, Schipper H M. Oxidative stress in diseases of the human cornea. Free Radical Biology and Medicine. 2008; 45(8): 1047-55.
12. DUA HS. Amniotic membrane transplantation. British Journal of Ophthalmology. 1999; 83(6):748-52.
13. Mamede A C, Botelho M F. Amniotic Membrane: Origin, Characterization and Medical

Applications: Springer Netherlands; 2015.
14. Sorsby A, Symons H. Amniotic membrane grafts in caustic burns of the eye: (Burns of the second degree). The British journal of ophthalmology. 1946; 30(6):337.
15. Gabler B, Lohmann C P. Hypopyon after repeated transplantation of human amniotic membrane onto the corneal surface. Ophthalmology. 2000; 107(7):1344-6.
16. Gauthier A S, Castelbou M, Gamier M B, Pizzuto J, Roux S, Gain P, et al. Corneal transplantation: study of the data of a regional eye bank for the year 2013 and analysis of the evolution of the adverse events reported in France since 2010. Cell and Tissue Banking. 2017; 18(1):83-9.
17. Nguyen P, Rue K, Heur M, Yiu S C. Ocular surface rehabilitation: Application of human amniotic membrane in high-risk penetrating keratoplasties. Saudi Journal of Ophthalmology. 2014; 28(3):198-202.
18. Qazi Y, Hamrah P. Corneal Allograft Rejection: Immunopathogenesis to Therapeutics. Journal of clinical & cellular immunology. 2013; 2013(Suppl 9):006.
19. R Mohan R, CK Tovey J, Gupta R, Sharma A, Tandon A. Decorin biology, expression, function and therapy in the cornea. Current molecular medicine. 2011; 11(2):110-28.
20. Du S, Wang S, Wu Q, Hu J, Li T. Decorin inhibits angiogenic potential of choroid-retinal endothelial cells by downregulating hypoxia-induced Met, Rac1, HIF-1α and VEGF expression in cocultured retinal pigment epithelial cells. Experimental eye research. 2013; 116:151-60.
21. Grant D S, Yenisey C, Rose R W, Tootell M, Santra M, Iozzo R V. Decorin suppresses tumor cell-mediated angiogenesis. Oncogene. 2002; 21(31):4765-77.
22. Iozzo R V, Buraschi S, Genua M, Xu S-Q, Solomides C C, Peiper S C, et al. Decorin antagonizes IGF receptor I (IGF-IR) function by interfering with IGF-IR activity and attenuating downstream signaling. Journal of Biological Chemistry. 2011; 286(40):34712-21.
23. Mohan R R, Tovey J C, Sharma A, Schultz G S, Cowden J W, Tandon A. Targeted decorin gene therapy delivered with adeno-associated virus effectively retards corneal neovascularization in vivo. PloS one. 2011; 6(10):e26432.
24. Zhu J-X, Goldoni S, Bix G, Owens R T, McQuillan D J, Reed C C, et al. Decorin evokes protracted internalization and degradation of the epidermal growth factor receptor via caveolar endocytosis. Journal of Biological Chemistry. 2005; 280(37):32468-79.
25. Kalamajski S, Oldberg A. The role of small leucine-rich proteoglycans in collagen fibrillogenesis. Matrix Biology. 2010; 29(4):248-53.
26. Nakamura N, Hart D A, Boorman R S, Kaneda Y, Shrive N G, Marchuk L L, et al. Decorin antisense gene therapy improves functional healing of early rabbit ligament scar with enhanced collagen fibrillogenesis in vivo. Journal of Orthopaedic Research. 2000; 18(4):517-23.
27. Reed C C, Iozzo R V. The role of decorin in collagen fibrillogenesis and skin homeostasis. Glycoconjugate journal. 2002; 19(4):249-55.
28. Zhang G, Chen S, Goldoni S, Calder B W, Simpson H C, Owens R T, et al. Genetic evidence for the coordinated regulation of collagen fibrillogenesis in the cornea by decorin and biglycan. Journal of Biological Chemistry. 2009; 284(13):8888-97.
29. Ahmed Z, Bansal D, Tizzard K, Surey S, Esmaeili M, Gonzalez A M, et al. Decorin blocks scarring and cystic cavitation in acute and induces scar dissolution in chronic spinal cord wounds. Neurobiology of disease. 2014; 64:163-76.
30. Logan A, Baird A, Berry M. Decorin attenuates gliotic scar formation in the rat cerebral hemisphere. Experimental neurology. 1999; 159(2):504-10.
31. Logan A, Frautschy S A, Gonzalez A-M, Sporn M B, Baird A. Enhanced expression of transforming growth factor β1 in the rat brain after a localized cerebral injury. Brain research. 1992; 587(2):216-25.
32. Rathore K, Nema R. An insight into ophthalmic drug delivery system. Int J Pharm Sci Drug Res. 2009; 1(1): 1-5.
33. Snibson G, Greaves J, Soper N, Tiffany J, Wilson C, Bron A. Ocular surface residence times of artificial tear solutions. Cornea. 1992; 11(4):288-93.
34. Farrés I F, Moakes R, Norton I. Designing biopolymer fluid gels: A microstructural approach. Food Hydrocolloids. 2014; 42:362-72.
35. Norton I, Jarvis D, Foster T. A molecular model for the formation and properties of fluid gels. International Journal of Biological Macromolecules. 1999; 26(4):255-61.
36. Wolf B, Frith W J, Singleton S, Tassieri M, Norton I T. Shear behaviour of biopolymer suspensions with spheroidal and cylindrical particles. Rheologica Acta. 2001; 40(3):238-47.
37. Barnes H A. A handbook of elementary rheology. Institute of Non-Newtonian Fluid Mechanics. University of Wales. 2000:5-130.
38. Graessley W W. The entanglement concept in polymer rheology. The entanglement concept in polymer rheology: Springer; 1974. p. 1-179.
39. Watanabe H. Viscoelasticity and dynamics of entangled polymers. Progress in Polymer Science. 1999; 24(9):1253-403.
40. Winter H. Can the gel point of a cross linking polymer be detected by the G′-G “crossover? Polymer Engineering & Science. 1987; 27(22):1698-702.
41. Karmakar M, Sun Y, Hise A G, Rietsch A, Pearlman E. Cutting edge: IL-1β processing during Pseudomonas aeruginosa infection is mediated by neutrophil serine proteases and is independent of NLRC4 and caspase-1. The Journal of Immunology. 2012; 189(9):4231-5.
42. Mishima S, Gasset A, Klyce S, Baum J. Determination of tear volume and tear flow. Investigative Ophthalmology & Visual Science. 1966; 5(3):264-76.
43. Sun Y, Karmakar M, Roy S, Ramadan R T, Williams S R, Howell S, et al. TLR4 and TLR5 on corneal macrophages regulate Pseudomonas aeruginosa keratitis by signaling through MyD88-dependent and-independent pathways. The Journal of Immunology. 2010; 185(7):4272-83.
44. Fleiszig S M, Evans D J. The pathogenesis of bacterial keratitis: studies with Pseudomonas aeruginosa. Clinical and Experimental Optometry. 2002; 85(5):271-8.
45. McClintic S M, Srinivasan M, Mascarenhas J, Greninger D A, Acharya N R, Lietman™, et al. Improvement in corneal scarring following bacterial keratitis. Eye. 2013; 27(3):443-6.
46. Botfield H, Gonzalez A M, Abdullah O, Skjolding A D, Berry M, McAllister J P, et al. Decorin prevents the development of juvenile communicating hydrocephalus. Brain. 2013; 136(9):2842-58.
47. Grisanti S, Szurman P, Warga M, Kaczmarek R, Ziemssen F, Tatar O, et al. Decorin modulates wound healing in experimental glaucoma filtration surgery: a pilot study. Investigative ophthalmology & visual science. 2005; 46(1):191-6.
48. Hill L J, Mead B, Blanch R J, Ahmed Z, De Cogan F, Morgan-Warren P J, et al. Decorin Reduces Intraocular Pressure and Retinal Ganglion Cell Loss in Rodents Through Fibrolysis of the Scarred Trabecular MeshworkDecorin Reduces Trabecular Meshwork Fibrosis. Investigative ophthalmology & visual science. 2015; 56(6):3743-57.
49. Mead R. The Design of Experiments: Statistical Principles for Practical Applications: Cambridge University Press; 1990.

1. A shear-thinning hydrogel composition comprising:
(i) 0.1 to 5 wt. % of a microgel particle-forming polymer; and
(ii) 0.5 to 100 mM of a monovalent and/or polyvalent metal ion salt as a cross-linking agent;
dispersed in an aqueous vehicle;
and wherein the hydrogel composition has a pH within a range of 3 to 8 and a viscosity of the hydrogel composition reduces when the hydrogel composition is exposed to shear.

2. The shear-thinning hydrogel composition according to claim 1, wherein the composition does not comprise collagen and/or fibrin.

3. The shear-thinning hydrogel composition according to claim 1, wherein the composition comprises 0.5 to 2.5 wt. % of the microgel particle-forming polymer.

4. The shear-thinning hydrogel composition according to claim 1, wherein the composition comprises 0.8 to 1.8 wt. % of the microgel particle-forming polymer.

5. The shear-thinning hydrogel composition according to claim 1, wherein the composition comprises 0.8 to 1.0 wt of the microgel particle-forming polymer.

6. The shear-thinning hydrogel composition according to claim 1, wherein the microgel particle-forming polymer is one or more polysaccharide microgel particle-forming polymers.

7. The shear-thinning hydrogel composition according to claim 1, wherein the microgel particle-forming polymer is selected from the group consisting of gellans, alginates, carrageenans, agarose, chitosan, gelatin and mixtures thereof.

8. The shear-thinning hydrogel composition according to claim 1, wherein the hydrogel composition is transparent or translucent and the microgel particle-forming polymer is selected from the group consisting of gellans, alginates, carrageenans and mixtures thereof.

9. The shear-thinning hydrogel composition according to claim 1, wherein the microgel particle-forming polymer is gellan.

10. The shear-thinning hydrogel composition according to claim 1, wherein the hydrogel composition does not comprise decorin.

11. The shear-thinning hydrogel composition according to claim 1, wherein the composition comprises 5 to 20 mM of the monovalent and/or polyvalent metal ion salt as the cross-linking agent.

12. The shear-thinning hydrogel composition according claim 1, wherein the composition comprises 5 to 15 mM of the monovalent and/or polyvalent metal ion salt as the cross-linking agent.

13. The shear-thinning hydrogel composition according to claim 1, wherein the composition comprises 8 to 12 mM of the monovalent and/or polyvalent metal ion salt as the cross-linking agent.

14. The shear-thinning hydrogel composition according to claim 1, wherein the composition comprises 10 mM of the monovalent and/or polyvalent metal ion salt as the cross-linking agent.

15. The shear-thinning hydrogel composition according to claim 1, wherein the microgel particle-forming polymer is gellan and the composition comprises 0.5 to 40 mM of the monovalent metal ion salt as the cross-linking agent.

16. The shear-thinning hydrogel composition according to claim 1, wherein the microgel particle-forming polymer is alginate and the composition comprises 0.5 to 40 mM of the polyvalent metal ion salt as the cross-linking agent.

17. The shear-thinning hydrogel composition according to claim 1, wherein the hydrogel composition has a pH within the range of 6 to 8 or 6.5 to 8.

18. The shear-thinning hydrogel composition according to claim 1, wherein the pH of the hydrogel composition is 7 to 7.5.

19. The shear-thinning hydrogel composition according to claim 1, wherein the viscosity of the hydrogel composition is:
(i) 1 Pa·s or greater when exposed to zero shear and the viscosity reduces when the hydrogel composition is subjected to shear;
(ii) 2 Pa·s or greater when exposed to zero shear and the viscosity reduces when the hydrogel composition is subjected to shear; or
(iii) 5 Pa·s or greater when exposed to zero shear and the viscosity reduces when the hydrogel composition is subjected to shear.

20-25. (canceled)

26. A method of making a shear-thinning hydrogel composition according to claim 1, the method comprising the steps of:
a) dissolving a microgel-forming polymer in an aqueous vehicle to form a polymer solution;
b) mixing the microgel-forming polymer solution formed in step (a) with an aqueous solution of a monovalent or polyvalent metal ion salt at a temperature above a gelling temperature of the microgel particle-forming polymer; and
c) cooling a resultant mixture from step b) to a temperature below the gelling temperature of the microgel particle-forming polymer to form the hydrogel composition.

27-56. (canceled)
